(19) United States
(12) Patent Application Publication
(10) Pub. No.: US 2004/0005614 A1
(43) Pub. Date: Jan. 8, 2004

(54) Methods for fragmentation, labeling and immobilization of nucleic acids

(76) Inventors: Nurith Kurn, Palo Alto, CA (US); Geoffrey A. Dafforn, Los Altos, CA (US)

Correspondence Address: MORRISON & FOERSTER LLP, 755 PAGE MILL RD, PALO ALTO, CA, 94304-1018, US

(21) Appl. No.: 10/441,663

(22) Filed: May 19, 2003

Related U.S. Application Data
(60) Provisional application No. 60/381,457, filed on May 17, 2002.

(51) Int. Cl. C12Q 1/68; C12P 19/34

(52) U.S. Cl. 435/6; 435/91.2

(57) ABSTRACT

The invention relates to methods for fragmentation and/or labeling and/or immobilization of nucleic acids. More particularly, the invention relates to methods for fragmentation and/or labeling and/or immobilization of nucleic acids comprising labeling and/or cleavage and/or immobilization at abasic sites.

### CROSS REFERENCE TO RELATED APPLICATIONS

This application claims the priority benefit of provisional application U.S. Serial No. 60/381,457, filed May 17, 2002, the contents of which is incorporated by reference in its entirety.

### TECHNICAL FIELD

The invention relates to methods for fragmentation and/or labeling and/or immobilization of nucleic acids. More particularly, the invention relates to methods for fragmentation and/or labeling and/or immobilization of nucleic acids comprising labeling and/or cleavage and/or immobilization at abasic sites.

### BACKGROUND ART

Fragmentation and labeling of nucleic acids are important for the analysis of genetic information contained within the nucleic acid sequence. For example, fragmentation and/or labeling are commonly required for detection of sequences by binding of a sample nucleic acid to complementary sequences immobilized on a surface, for example, on a microarray. Cleavage of sample nucleic acid into small fragments (e.g., 50-100 base pairs) facilitates diffusion of nucleic acid onto the surface, and may facilitate hybridization. It is known, for example, that steric and charge hindrance effects increase with the size of nucleic acids that are hybridized. Moreover, cleavage of sample nucleic acids into small fragments may ensure that two sequences of interest in the sample do not appear to bind to the same template nucleic acid simply by virtue of their proximity on the test nucleic acid. Cleavage of nucleic acids also facilitates detection of hybridized nucleic acid when, as in many detection methods, the size of the signal is proportional to the size of the bound fragment and thus, control of fragment size is desirable. Labeling of nucleic acids is necessary in many methods of nucleic acid analysis because there are presently few techniques for direct detection of unlabeled nucleic acid with the requisite sensitivity for analysis on chips. Methods for fragmenting and/or labeling nucleic acids are known in the art. See, e.g., U.S. Pat. Nos. 5,082,830; 4,996,143; 5,688,648; 6,326,142; WO02/090584, and references cited therein.

Immobilization of nucleic acids to create, for example, microarrays or tagged analytes, is useful for, e.g., detection and analysis of nucleic acids and tagged analytes. Methods for immobilizing nucleic acids are known in the art. See, e.g., U.S. Pat. Nos. 5,667,979; 6,077,674; 6,280,935; and references cited therein.

There is a serious need for improved methods for labeling and/or fragmenting and/or immobilizing nucleic acids to a surface, for example a microarray.

All references cited herein, including patent applications and publications, are incorporated by reference in their entirety.

### SUMMARY OF THE INVENTION

The invention provides novel methods and kits for labeling and/or fragmenting and/or immobilizing polynucleotides to a substrate.

In one aspect, the invention provides methods for fragmenting and labeling a polynucleotide, said method comprising: (a) synthesizing a polynucleotide from a template in the presence of at least one non-canonical nucleotide, whereby a polynucleotide comprising a non-canonical nucleotide is generated; (b) contacting the synthesized polynucleotide with an enzyme capable of cleaving a base portion of the non-canonical nucleotide from the synthesized polynucleotide (i.e. cleaving a base portion of a non-canonical nucleotide with an enzyme capable of cleaving a base portion of a non-canonical nucleotide), whereby an abasic site is created; (c) cleaving a phosphodiester backbone at the abasic site; and (d) contacting the synthesized polynucleotide with an agent capable of labeling the abasic site (i.e. labeling an abasic site), whereby a labeled polynucleotide fragment is generated.

In one aspect, the invention provides methods for fragmenting and labeling a polynucleotide, said method comprising (a) contacting a polynucleotide comprising a non-canonical nucleotide with an enzyme capable of cleaving a base portion of the non-canonical nucleotide, whereby an abasic site is created, wherein the polynucleotide comprising a non-canonical nucleotide is synthesized from a template in the presence of at least one non-canonical nucleotide; (b) cleaving a phosphodiester backbone at the abasic site; and (c) contacting the polynucleotide with an agent capable of labeling the abasic site (i.e. labeling at the abasic site); whereby a labeled polynucleotide fragment is generated.

In another aspect, the invention provides methods for fragmenting and labeling a polynucleotide, said method comprising (a) cleaving a phosphodiester backbone at an abasic site of a polynucleotide comprising the abasic site, wherein the polynucleotide comprising the abasic site is generated by contacting a polynucleotide comprising a non-canonical nucleotide with an enzyme capable of cleaving a base portion of the non-canonical nucleotide, whereby an abasic site is created, wherein the polynucleotide comprising a non-canonical nucleotide is synthesized from a template in the presence of at least one non-canonical nucleotide; and (b) contacting the polynucleotide with an agent capable of labeling the abasic site; whereby labeled fragments of the polynucleotide are generated.

In another aspect, the invention provides methods for fragmenting and labeling a polynucleotide, said method comprising contacting a polynucleotide comprising an abasic site with an agent capable of labeling the abasic site; wherein the polynucleotide is generated by cleaving a phosphodiester backbone at an abasic site of a polynucleotide comprising the abasic site, wherein the polynucleotide comprising the abasic site is generated by contacting a polynucleotide comprising a non-canonical nucleotide with an enzyme capable of cleaving a base portion of the non-canonical nucleotide, whereby an abasic site is created, wherein the polynucleotide comprising a non-canonical nucleotide is synthesized from a template in the presence of at least one non-canonical nucleotide; whereby labeled fragments of the polynucleotide are generated.

In another aspect, the invention provides method for fragmenting and labeling a polynucleotide comprising: (a) incubating a reaction mixture, said reaction mixture comprising: (i) a template and (ii) a non-canonical nucleotide; wherein the incubation is under conditions that permit formation of a polynucleotide comprising a non-canonical nucleotide; (b) incubating a reaction mixture, said reaction mixture comprising: (i) a polynucleotide comprising a non-canonical nucleotide; and (ii) an agent capable of specifically cleaving a base portion of a non-canonical nucleotide; wherein the incubation is under conditions that permit cleavage of the base portion of the non-canonical nucleotide, whereby a polynucleotide comprising an abasic site is generated; (c) incubating a reaction mixture, said reaction mixture comprising: (i) a polynucleotide comprising an abasic site; and (ii) an agent capable of effecting (generally, specific) cleavage of a phosphodiester backbone at the abasic site; wherein the incubation is under conditions that permit cleavage of the phosphodiester backbone at the abasic site; whereby fragments of the polynucleotide are generated; (d) incubating a reaction mixture, said reaction mixture comprising: (i) a polynucleotide comprising an abasic site; and (ii) an agent capable of labeling the abasic site; wherein the incubation is under conditions that permit labeling at the abasic site; whereby labeled fragments are generated.

In another aspect, the invention provides methods for labeling and fragmenting a polynucleotide, said method comprising: (a) incubating a reaction mixture, said reaction mixture comprising: (i) a template and (ii) a non-canonical nucleotide; wherein the incubation is under conditions that permit formation of a polynucleotide comprising a non-canonical nucleotide; (b) incubating a reaction mixture, said reaction mixture comprising: (i) the polynucleotide comprising the non-canonical polynucleotide; (ii) an enzyme capable of cleaving a base portion of a non-canonical nucleotide; (iii) an agent capable of cleaving a polynucleotide at the abasic site; wherein the incubation is under conditions that permit cleavage of a base portion of a non-canonical nucleotide and optionally, cleavage of the polynucleotide at the abasic site; whereby fragments of the polynucleotide are generated; and (b) incubating a reaction mixture, said reaction mixture comprising: (i) a polynucleotide fragment comprising an abasic site; and (ii) an agent capable of labeling the abasic site; wherein the incubation is under conditions that permit labeling at the abasic site; whereby a labeled fragment is generated.

In another aspect, the invention provides the invention provides methods for labeling and fragmenting a polynucleotide, said method comprising (a) incubating a reaction mixture, said reaction mixture comprising: (i) a template; (ii) a non-canonical nucleotide; (iii) an enzyme capable of cleaving a base portion of a non-canonical nucleotide; and (iv) an agent capable of cleaving a polynucleotide at the abasic site; wherein the incubation is under conditions that permit formation of a polynucleotide comprising a non-canonical nucleotide, cleavage of a base portion of a non-canonical nucleotide and cleavage of the polynucleotide at the abasic site; whereby fragments of the polynucleotide are generated; and (b) incubating a reaction mixture, said reaction mixture comprising: (i) a polynucleotide fragment comprising an abasic site or optionally, fragments of a polynucleotide comprising an abasic site; and (ii) an agent capable of labeling the abasic site; wherein the incubation is under conditions that permit labeling at the abasic site; whereby a labeled fragment is generated.

As is evident to one skilled in the art, aspects that refer to combining and incubating the resultant mixture also encompasses method embodiments which comprise incubating the various mixtures (in various combinations and/or subcombinations) so that the desired products are formed. The reaction mixtures may be combined (thus reducing the number of incubations) in any way, with one or more reaction mixtures above combined.

Accordingly, in some embodiments, synthesizing a polynucleotide comprising a non-canonical nucleotide and cleaving a base portion of a non-canonical nucleotide are conducted in the same reaction mixture. In other embodiments, synthesizing a polynucleotide comprising a non-canonical nucleotide, cleaving a base portion of a non-canonical nucleotide, and cleaving the backbone at an abasic site are conducted in the same reaction mixture. In still another embodiment, synthesizing a polynucleotide comprising a non-canonical nucleotide and cleaving a base portion of a non-canonical nucleotide are conducted in the same reaction mixture, and cleaving the backbone at an abasic site and labeling at the abasic site are conducted in same reaction mixture. In another embodiment, synthesizing a polynucleotide comprising a non-canonical nucleotide, cleaving a base portion of a non-canonical nucleotide, cleaving the backbone at an abasic site, and labeling at the abasic site are conducted in same reaction mixture. In other embodiments, synthesizing a polynucleotide comprising a non-canonical nucleotide, cleaving a base portion of a non-canonical nucleotide, cleaving the backbone at an abasic site, and labeling at the abasic site are conducted in same reaction mixture. In other embodiments, cleaving a base portion of a non-canonical nucleotide, and cleaving the backbone at an abasic site are conducted in the same reaction mixture. In other embodiments, cleaving the backbone at an abasic site, and labeling at the abasic site are conducted in the same reaction mixture. In another embodiment, cleaving a base portion of a non-canonical nucleotide and labeling at the abasic site are conducted in the same reaction mixture. In another embodiment, synthesizing a polynucleotide comprising a non-canonical nucleotide, cleaving a base portion of a non-canonical nucleotide, and labeling at an abasic site are conducted in the same reaction mixture. It is understood that any combination of these incubation steps, and any single incubation step, to the extent that the incubation is performed as part of any of the methods described herein, fall within the scope of the invention. As explained herein, labeling can occur before fragmentation (i.e. cleavage of the phosphodiester backbone at an abasic site), fragmentation can occur before labeling, or fragmentation and labeling can occur simultaneously.

In another aspect, the invention provides methods for labeling a polynucleotide, said method comprising: (a) synthesizing a polynucleotide from a template in the presence of at least one non-canonical nucleotide, whereby a polynucleotide comprising a non-canonical nucleotide is generated; (b) contacting the synthesized polynucleotide with an enzyme capable of effecting cleavage of a base portion of the non-canonical nucleotide from the synthesized polynucleotide, whereby an abasic site is created; (c) contacting the synthesized polynucleotide with an agent capable of labeling the abasic site; whereby the synthesized polynucleotide is labeled.

In one aspect, the invention provides methods for labeling a polynucleotide, said method comprising: (a) contacting a polynucleotide comprising a non-canonical nucleotide with an enzyme capable of cleaving a base portion of the non-canonical nucleotide, whereby an abasic site is created, wherein the polynucleotide comprising a non-canonical nucleotide is synthesized from a template in the presence of at least one non-canonical nucleotide; (b) contacting the polynucleotide with an agent capable of labeling the abasic site; whereby the polynucleotide is labeled.

In another aspect, the invention provides methods for labeling a polynucleotide, said method comprising contacting a polynucleotide comprising an abasic site with an agent capable of labeling the abasic site; wherein the polynucleotide comprising the abasic site is generated by contacting a polynucleotide comprising a non-canonical nucleotide with an enzyme capable of cleaving a base portion of the non-canonical nucleotide, whereby an abasic site is created, wherein the polynucleotide comprising a non-canonical nucleotide is synthesized from a template in the presence of at least one non-canonical nucleotide; whereby the polynucleotide is labeled.

In another aspect, the invention provides methods for labeling a polynucleotide, said method comprising: (a) preparing an aminooxy derivative of Alexa Fluor 555; and (b) contacting a polynucleotide comprising an abasic site (prepared using methods described herein) with the aminooxy derivative of Alexa Fluor 555; whereby the polynucleotide is labeled. In another aspect, the invention provides methods for labeling a polynucleotide comprising contacting a polynucleotide comprising an abasic site (prepared using methods described herein) with an aminooxy derivative of Alex Fluor 555; whereby the polynucleotide is labeled.

In some embodiments of the methods of generating polynucleotides immobilized to a surface (i.e., a substrate), the polynucleotide comprising an abasic site is labeled at an abasic site.

In another aspect, the invention provides methods for labeling a polynucleotide comprising: (a) incubating a reaction mixture, said reaction mixture comprising: (i) a template and (ii) a non-canonical nucleotide; wherein the incubation is under conditions that permit formation of a polynucleotide comprising a non-canonical nucleotide; (b) incubating a reaction mixture, said reaction mixture comprising: (i) a polynucleotide comprising a non-canonical nucleotide; and (ii) an agent capable of specifically cleaving a base portion of a non-canonical nucleotide; wherein the incubation is under conditions that permit cleavage of the base portion of the non-canonical nucleotide, whereby a polynucleotide comprising an abasic site is generated; (c) incubating a reaction mixture, said reaction mixture comprising: (i) a polynucleotide comprising an abasic site; and (ii) an agent capable of labeling the abasic site; wherein the incubation is under conditions that permit labeling at the abasic site; whereby labeled polynucleotides are generated.

As is evident to one skilled in the art, aspects that refer to combining and incubating the resultant mixture also encompasses method embodiments which comprise incubating the various mixtures (in various combinations and/or subcombinations) so that the desired products are formed. The reaction mixtures may be combined (thus reducing the number of incubations) in any way, with one or more reaction mixtures above combined.

Accordingly, in some embodiments, synthesizing a polynucleotide comprising a non-canonical nucleotide and cleaving a base portion of a non-canonical nucleotide are conducted in the same reaction mixture. In other embodiments, synthesizing a polynucleotide comprising a non-canonical nucleotide, cleaving a base portion of a non-canonical nucleotide, and labeling at the abasic site are conducted in same reaction mixture. In other embodiments, cleaving a base portion of a non-canonical nucleotide, and labeling at the abasic site are conducted in same reaction mixture. It is understood that any combination of these incubation steps, and any single incubation step, to the extent that the incubation is performed as part of any of the methods described herein, fall within the scope of the invention.

In another aspect, the invention provides methods for labeling and optionally fragmenting a polynucleotide, said method comprising: (a) synthesizing a polynucleotide from a polynucleotide template in the presence of a non-canonical nucleotide, whereby a polynucleotide comprising the non-canonical nucleotide is generated; (b) cleaving a base portion of a non-canonical nucleotide from the synthesized polynucleotide with an enzyme capable of cleaving the base portion of the non-canonical nucleotide, whereby an abasic site is generated; (c) optionally, cleaving a phosphodiester backbone of the polynucleotide comprising the abasic site at the abasic site; and (d) labeling the polynucleotide or the fragment of the polynucleotide at the abasic site; whereby a labeled polynucleotide, or optionally, a labeled polynucleotide fragment is generated.

In another aspect, the invention provides methods for labeling and optionally fragmenting a polynucleotide, said method comprising: (a) incubating a reaction mixture, said reaction mixture comprising: (i) a polynucleotide template; and (ii) a non-canonical nucleotide; wherein the incubation is under conditions that permit synthesis of a polynucleotide comprising the non-canonical nucleotide, whereby a polynucleotide comprising the non-canonical nucleotide is generated; (b) incubating a reaction mixture, said reaction mixture comprising: (i) the polynucleotide comprising the non-canonical nucleotide; and (ii) an enzyme capable of cleaving a base portion of the non-canonical nucleotide, wherein the incubation is under conditions that permit cleavage of the base portion of the non-canonical nucleotide, whereby a polynucleotide comprising an abasic site is generated; (c) optionally incubating a reaction mixture, said reaction mixture comprising: (i) the polynucleotide comprising the abasic site; and (ii) an agent capable of cleaving a phosphodiester backbone of the polynucleotide comprising the abasic site at the abasic site, wherein the incubation is under conditions that permit cleavage of the phosphodiester backbone of the polynucleotide at the abasic site, whereby a fragment of the polynucleotide is generated; (d) incubating a reaction mixture, said reaction mixture comprising: (i) the polynucleotide comprising the abasic site or optionally, the fragment of the polynucleotide comprising the abasic site; and (ii) an agent capable of labeling the abasic site, wherein the incubation is under conditions that permit labeling at the abasic site; whereby a labeled polynucleotide or optionally, a labeled polynucleotide fragment, is generated.

In another aspect, the invention provides methods for labeling and optionally fragmenting a polynucleotide, said method comprising (a) incubating a reaction mixture, said reaction mixture comprising: (i) the polynucleotide comprising the non-canonical polynucleotide of step (a) of claim 1; (ii) an enzyme capable of cleaving a base portion of the non-canonical nucleotide; and (iii) optionally, an agent capable of cleaving a phosphodiester backbone of the polynucleotide comprising the abasic site at the abasic site, wherein the incubation is under conditions that permit cleavage of the base portion of the non-canonical nucleotide and optionally, cleavage of the phosphodiester backbone of the polynucleotide at the abasic site; whereby polynucleotide comprising the abasic site, or optionally, a fragment of the polynucleotide comprising the abasic site, is generated; and (b) incubating a reaction mixture, said reaction mixture comprising: (i) the polynucleotide comprising the abasic site or optionally, the fragment of the polynucleotide comprising the abasic site; and (ii) an agent capable of labeling the abasic site, wherein the incubation is under conditions that permit labeling at the abasic site, whereby a labeled polynucleotide or optionally, a labeled fragment of the polynucleotide, is generated.

In another aspect, the methods of the invention provide methods for generating polynucleotides immobilized to a surface, said methods comprising immobilizing a polynucleotide comprising an abasic site to a surface, wherein the polynucleotide is immobilized at the abasic site. In some embodiments, the polynucleotide comprising an abasic site is generated by contacting a polynucleotide comprising a non-canonical nucleotide with an enzyme capable of cleaving a base portion of the non-canonical nucleotide from the polynucleotide, whereby an abasic site is created. In further embodiments, the polynucleotide comprising a non-canonical nucleotide is synthesized from a template in the presence of at least one non-canonical nucleotide.

In another aspect, the methods of the invention provide methods for generating polynucleotides immobilized to a surface, said method comprising: (a) contacting a polynucleotide comprising a non-canonical nucleotide with an enzyme capable of cleaving a base portion of the non-canonical nucleotide from the polynucleotide, whereby an abasic site is created; (b) optionally cleaving a phosphodiester backbone at the abasic site; whereby fragments of the polynucleotide are generated; and (c) immobilizing the polynucleotide comprising an abasic site, or fragments thereof, to a surface, wherein the polynucleotide is immobilized at an abasic site. In some embodiments, the polynucleotide is synthesized from a template in the presence of at least one non-canonical nucleotide.

In another aspect, the invention provides methods for generating a polynucleotide immobilized to a surface, said methods comprising: (a) cleaving a phosphodiester backbone at an abasic site of a polynucleotide comprising the abasic site; whereby fragments of the polynucleotide are generated; and (b) immobilizing the fragments of the polynucleotide to a surface, wherein the polynucleotide is immobilized at the abasic site. In some embodiments, the polynucleotide comprising an abasic site is generated by contacting a polynucleotide comprising a non-canonical nucleotide with an enzyme capable of cleaving a base portion of the non-canonical nucleotide from the polynucleotide, whereby an abasic site is created. In further embodiments, the polynucleotide comprising a non-canonical nucleotide is synthesized from a template in the presence of at least one non-canonical nucleotide.

In another aspect, the invention provides methods for immobilizing a polynucleotide comprising: (a) incubating a reaction mixture, said reaction mixture comprising: (i) a polynucleotide comprising a non-canonical nucleotide; and (ii) an agent capable of specifically cleaving a base portion of a non-canonical nucleotide; wherein the incubation is under conditions that permit cleavage of the base portion of the non-canonical nucleotide, whereby a polynucleotide comprising an abasic site is generated; (b) optionally incubating a reaction mixture, said reaction mixture comprising: (i) a polynucleotide comprising an abasic site; and (ii) an agent capable of effecting specific cleavage of a phosphodiester backbone at the abasic site; wherein the incubation is under conditions that permit cleavage of the phosphodiester backbone at the abasic site; whereby fragments of the polynucleotide are generated; (c) incubating a reaction mixture, said reaction mixture comprising: (i) a polynucleotide, or fragment thereof, comprising an abasic site; and (ii) a surface (i.e., a substrate); and (iii) an agent capable of immobilizing the polynucleotide, or fragment thereof, comprising the abasic site to the surface at the abasic site; wherein the incubation is under conditions that permit immobilization of the polynucleotide, or fragment thereof, to the surface at the abasic site; whereby immobilized polynucleotides, or fragments thereof, are generated. In some embodiments, the polynucleotide is synthesized from a template in the presence of at least one non-canonical nucleotide.

As is evident to one skilled in the art, aspects that refer to combining and incubating the resultant mixture also encompasses method embodiments which comprise incubating the various mixtures (in various combinations and/or subcombinations) so that the desired products are formed. The reaction mixtures may be combined (thus reducing the number of incubations) in any way, with one or more reaction mixtures above combined. It is understood that any combination of these incubation steps, and any single incubation step, to the extent that the incubation is performed as part of any of the methods described herein, fall within the scope of the invention

Various embodiments of the methods of the inventions are described herein. For example, in embodiments involving synthesis of a polynucleotide comprising a non-canonical nucleotide from a template, the synthesizing can be by PCR, primer extension, reverse transcription, DNA replication, strand displacement amplification (SDA), multiple displacement amplification (MDA), and the like. In some embodiments, the polynucleotide is synthesized using single primer isothermal amplification, for example, wherein a polynucleotide sequence complementary to a target polynucleotide is amplified using methods comprising the following steps of: (a) hybridizing a single stranded DNA template comprising the target sequence with a composite primer, said composite primer comprising a RNA portion and a 3′ DNA portion; (b) optionally hybridizing a polynucleotide comprising a termination polynucleotide sequence to a region of the template which is 5′ with respect to hybridization of the composite primer to the template; (c) extending the composite primer with DNA polymerase; and (d) cleaving the RNA portion of the annealed composite primer with an enzyme that cleaves RNA from an RNA/DNA hybrid such that another composite primer hybridizes to the template and repeats primer extension by strand displacement, whereby multiple copies of the complementary sequence of the target sequence are produced. In another embodiment, the polynucleotide is synthesized using methods comprising the following steps of: (a) extending a composite primer in a complex comprising (i) a polynucleotide template; and (ii) the composite primer, said composite primer comprising an RNA portion and a 3′ DNA portion, wherein the polynucleotide template is hybridized to the composite primer; and (b) cleaving the RNA portion of the annealed composite primer with an enzyme that cleaves RNA from an RNA/DNA hybrid such that another composite primer hybridizes to the template and repeats primer extension by strand displacement, whereby multiple copies of the complementary sequence of the target sequence are produced. In some embodiments, the RNA portion of the composite primer is 5′ with respect to the 3′ DNA portion, the 5′ RNA portion is adjacent to the 3′ DNA portion, the RNA portion of the composite primer consists of about 10 to about 20 nucleotides and the DNA portion of the composite primer consists of about 7 to about 20 nucleotides.

In other embodiments, the polynucleotide is synthesized using Ribo-SPIA™, for example wherein multiple copies of a polynucleotide sequence complementary to an RNA sequence of interest (template) are generated using methods comprising the following steps of: (a) extending a first primer hybridized to a target RNA with an RNA-dependent DNA polymerase, wherein the first primer is a composite primer comprising an RNA portion and a 3′ DNA portion, whereby a complex comprising a first primer extension product and the target RNA is produced; (b) cleaving RNA in the complex of step (b) with an enzyme that cleaves RNA from an RNA/DNA hybrid; (c) extending a second primer hybridized to the first primer extension product with a DNA-dependent DNA polymerase and a RNA-dependent DNA polymerase, whereby a second primer extension product is produced to form a complex of first and second primer extension products; (d) cleaving RNA from the composite primer in the complex of first and second primer extension products with an enzyme that cleaves RNA from an RNA/DNA hybrid such that a composite primer hybridizes to the second primer extension product, wherein the composite primer comprises an RNA portion and a 3′ DNA portion; (e) extending the composite primer hybridized to the second primer extension product with a DNA-dependent DNA polymerase; whereby said first primer extension product is displaced, and whereby multiple copies of a polynucleotide sequence complementary to the RNA sequence of interest are generated. In some embodiment, RNA in a complex of step (b) is cleaved with an agent (such as heat or basic conditions) that cleaves RNA from an RNA/DNA hybrid.

In some embodiments, the polynucleotide that is synthesized is single stranded. In other embodiments, the polynucleotide that is synthesized is double-stranded. In still other embodiments, the polynucleotide that is synthesized is partially double stranded. In still other embodiments, the polynucleotide that is synthesized comprises a cDNA. In still other embodiments, the template comprises RNA, mRNA, genomic DNA, plasmid DNA, synthetic DNA, cDNA. In other embodiments, the template comprises a cDNA library, a genomic library, or a subtractive hybridization library. In still other embodiments, the polynucleotide comprising a non-canonical nucleotide is synthesized using a labeled primer. In still other embodiments, the polynucleotide comprising a non-canonical nucleotide is synthesized using a primer comprising a non-canonical nucleotide. In other embodiments, the polynucleotide comprising a non-canonical nucleotide is synthesized in the presence of two or more different non-canonical nucleotides, whereby a polynucleotide comprising two or more different non-canonical nucleotide is synthesized. In other embodiments, the polynucleotide comprising a non-canonical nucleotide is synthesized from two or more different polynucleotide templates.

In some embodiments, the non-canonical nucleotide is dUTP. In other embodiments, the non-canonical nucleotide is dUTP and the enzyme capable of cleaving a base portion of the non-canonical nucleotide from the synthesized polynucleotide is Uracil N-Glycosylase (interchangeably termed “UNG”).

In embodiments involving fragmentation, the phosphodiester backbone can be cleaved by an agent, such as an enzyme or an amine, capable of effecting cleavage of a phosphodiester backbone at an abasic site. In some embodiments, the enzyme is E. coli Endonuclease IV. In other embodiments, the agent is N,N′-dimethylethylenediamine. In still other embodiments, the agent is heat, basic conditions, or acidic conditions.

In embodiments involving fragmentation, the fragments can be about 10, about 15, about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In some embodiments, the fragments can be at least about 15, about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In other embodiments, the fragments can be less than about 15, about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. It is understood that these fragment lengths may represent an average size in the population of fragments generated using the methods of the invention.

In some embodiments, the fragments comprise an abasic site at the 3′ end (terminus). In other embodiments, the fragments comprise an abasic site at the 5′ end (terminus). In still other embodiments, the fragments comprise both abasic sites at the 3′ ends and abasic sites at the 5′ ends. It is understood that a polynucleotide fragment may additionally comprise internal abasic sites (i.e., abasic sites that are not at the 3′ or 5′ end of the fragment), as when, for example, fragmentation does not occur at every abasic site in a polynucleotide.

In embodiments involving labeling, the polynucleotide comprising a non-canonical nucleotide, or fragments thereof, is labeled at an abasic site, whereby. a polynucleotide (or polynucleotide fragment) comprising a label is generated. In some embodiments, the polynucleotide, or fragments thereof, comprising an abasic site is contacted with an agent capable of labeling the abasic site. In various embodiments, the detectable moiety (label) is covalently or non-covalently associated or directly or indirectly associated with an abasic site. In some embodiments, the label is directly or indirectly detectable. In some embodiments, the label comprises an organic molecule, a hapten, or a particle (such as a polystyrene bead). In some embodiments, the label is detected using antibody binding, biotin binding, or via fluorescence or enzyme activity. In some embodiments, the detectable signal is amplified. In some embodiments, the detectable moiety comprises an organic molecule. In some embodiments, the label reacts with an aldehyde residue at the abasic site. In other embodiments, the label comprises a reactive group selected from: a hydrazine, or a hydroxylamine. In some embodiments, the label is 5-(((2-(carbohydrazino)-methyl)thio)acetyl)aminofluorescein, aminooxyacetyl hydrazide (“FARP”). In another embodiment, the label is N-(aminooxyacetyl)-N′-(D-biotinoyl) hydrazine, trifluoroacetic acid salt (“ARP”). In yet another embodiment, the label is Alexa 555. In yet another embodiment, the label is an aminooxy derivative of Alexa Fluor 555.

In another aspect, the invention provides an aminooxy derivative of Alexa Fluor 555, wherein the aminooxy derivative is generated as disclosed herein.

In some embodiments involving immobilization, the polynucleotide or fragment thereof, is immobilized on a substrate (used interchangeably herein with “surface”) at the abasic site. In some embodiments, the substrate comprises a solid or semi-solid support. In some embodiments, the substrate is a microarray. In other embodiments, the microarray comprises at least one probe immobilized on a substrate fabricated from a material selected from the group consisting of paper, glass, ceramic, plastic, polypropylene, polystyrene, nylon, polyacrylamide, nitrocellulose, silicon (and other metals), and optical fiber. In still other embodiments, the polynucleotide, or fragment thereof, is immobilized on the substrate in a two-dimensional configuration or a three-dimensional configuration comprising pins, rods, fibers, tapes, threads, beads, particles, microtiter wells, capillaries, and cylinders.

In other embodiments, a substrate which is an analyte is selected from the group consisting of a protein, a polypeptide, a peptide, a carbohydrate, an organic molecule, an inorganic molecule, a cell, a microorganism, and fragments and products thereof. In other embodiments, the analyte is selected from the group consisting of a polypeptide, an antibody, an organic molecule and an inorganic molecule.

The methods are applicable to generating labeled polynucleotides, labeled polynucleotide fragments, or immobilized polynucleotides (or fragments thereof), or labeled immobilized polynucleotides (or fragments thereof) from any polynucleotide target, including, for example, mRNA, genomic DNA, cDNA, cloned DNA, and synthetic DNA. One or more steps may be combined and/or performed sequentially (often in any order, as long as the requisite product(s) are able to be formed), and, as is evident, the invention includes various combinations of the steps described herein. It is also evident, and is described herein, that the invention encompasses methods in which the initial, or first, step is any of the steps described herein. Methods of the invention encompass embodiments in which later, “downstream” steps are an initial step. The reaction mixtures may be combined (thus reducing the number of incubations) in any way, with one or more reaction mixtures above combined. Accordingly, in some embodiments, synthesizing a polynucleotide comprising a non-canonical nucleotide and cleaving a base portion of a non-canonical nucleotide are conducted in the same reaction mixture. In other embodiments, synthesizing a polynucleotide comprising a non-canonical nucleotide, cleaving a base portion of a non-canonical nucleotide, and labeling at the abasic site are conducted in same reaction mixture. In other embodiments, synthesizing a polynucleotide comprising a non-canonical nucleotide, cleaving a base portion of a non-canonical nucleotide, labeling at the abasic site are conducted in same reaction mixture, and immobilizing at an abasic site are conducted in the same reaction mixture. In other embodiments, synthesizing a polynucleotide comprising a non-canonical nucleotide, cleaving a base portion of a non-canonical nucleotide, and immobilizing at an abasic site are conducted in the same reaction mixture. In other embodiments, cleaving a base portion of a non-canonical nucleotide, and labeling at the abasic site are conducted in same reaction mixture. In other embodiments, cleaving a base portion of a non-canonical nucleotide, and immobilizing at the abasic site are conducted in same reaction mixture. In other embodiments, labeling at an abasic site and immobilizing at an abasic site are conducted in the same reaction mixture. It is understood that any combination of these incubation steps, and any single incubation step, to the extent that the incubation is performed as part of any of the methods described herein, fall within the scope of the invention.

The invention also provides methods which employ (usually, analyze) the products of the labeling and/or labeling and/or immobilization methods of the invention, such as methods of detecting the presence or absence of nucleic acid sequence mutations; methods to characterize (for example, detect presence or absence of and/or quantify) a polynucleotide template; methods of preparing a hybridization probe; methods of hybridization using the hybridization probes; methods of detection using the hybridization probe; methods of determining a gene expression profile; method of comparative hybridization; methods of identifying a polynucleotide; and methods of preparing a subtractive hybridization probe.

In one aspect, the invention provides methods of detecting presence or absence of a mutation in a template, comprising: (a) generating a labeled polynucleotide, or fragments thereof, by any of the methods described herein; and (b) analyzing the labeled polynucleotide, or fragments thereof, whereby presence or absence of a mutation is detected. In some embodiments, the labeled polynucleotide, or fragments thereof, is compared to a labeled reference template, or fragments thereof. Step (b) of analyzing the labeled polynucleotide, or fragments thereof, whereby presence or absence of a mutation is detected, can be performed by any method known in the art. In some embodiments, probes for detecting mutations are provided as a microarray.

In another aspect, the invention provides methods of characterizing a template, comprising: (a) generating a labeled polynucleotide, or fragments thereof, by any of the methods described herein; and (b) analyzing the polynucleotide, or fragments thereof. Step (b) of analyzing the labeled polynucleotide, or fragments thereof, can be performed by any method known in the art or described herein, for example by detecting and/or quantifying labeled polynucleotide, or fragments thereof, that are hybridized to a probe. In some embodiments, the at least one probe is provided as a microarray. The microarray can comprise at least one probe immobilized on a solid or semi-solid substrate fabricated from a material selected from the group consisting of paper, glass, ceramics, plastic, polypropylene, polystyrene, nylon, polyacrylamide, nitrocellulose, silicon, other metals, and optical fiber. A probe can be immobilized on the solid or semi-solid substrate in a two-dimensional configuration or a three-dimensional configuration comprising pins, rods, fibers, tapes, threads, beads, particles, microtiter wells, capillaries, and cylinders. In some embodiments, step (b) of analyzing the labeled polynucleotide, or fragment thereof, comprises determining amount of said products, whereby the amount of the template present in a sample is quantified. In other embodiments, step (b) of analyzing the labeled polynucleotide, or fragment thereof, comprises determining the sequence of the labeled polynucleotide (or fragments thereof) for example, using sequencing by hybridization.

In another aspect, the invention provides methods for identifying a polynucleotide, comprising: (a) generating a labeled polynucleotide, or fragments thereof, from a polynucleotide template by any of the methods described herein; and (b) analyzing the polynucleotide, or fragments thereof, whereby the polynucleotide is identified. In some embodiments, step (b) of identifying the polynucleotide comprises hybridizing the labeled polynucleotide or fragments thereof to at least one probe.

In another aspect, the invention provides methods of determining gene expression profile in a sample, said method comprising: (a) generating a labeled polynucleotide, or fragments thereof, by any of the methods described herein; and (b) determining amount of labeled polynucleotide, or fragments thereof, generated from each template polynucleotide, wherein each said amount is indicative of amount of each template in the sample, whereby the gene expression profile in the sample is determined.

Any of these applications can use any of the methods (including various components and various embodiments of any of the components) as described herein.

The invention also provides compositions, kits, complexes, reaction mixtures and systems comprising various components (and various combinations of the components) used in the methods described herein.

### BRIEF DESCRIPTION OF THE DRAWINGS

FIG. 1: shows a diagrammatic illustration of a method for fragmenting and labeling a nucleic acid. “R” indicates a nucleotide residue.

FIG. 2: shows a diagrammatic illustration of a method for labeling a nucleic acid. “R” indicates a nucleotide residue.

FIG. 3: shows a diagrammatic illustration of a method for immobilizing a nucleic acid to a surface. “R” indicates a nucleotide residue.

FIG. 4: shows a gel showing fragmented labeled polynucleotide fragments generated by (1) creating an abasic site by cleaving a base portion of a non-canonical nucleotide present in an oligonucleotide, (2) cleaving the phosphodiester backbone at the abasic site, and (3) labeling the abasic site using an agent capable of specifically labeling an abasic site.

FIG. 5: shows a gel showing labeled polynucleotides generated by (1) creating an abasic site by cleaving a base portion of a non-canonical nucleotide present in an oligonucleotide, and (2) labeling the abasic site using an agent capable of specifically labeling an abasic site.

FIG. 6: shows a gel showing labeled polynucleotide fragments generated according to the fragmentation and labeling methods of the invention, wherein the synthesized polynucleotides were amplified using the single primer amplification methods described in Kurn, U.S. Patent Publication No. 2003/0087251 A1, which is hereby incorporated by reference in its entirety.

FIG. 7: shows an electropherogram showing labeled polynucleotide fragments generated according to the fragmentation and labeling methods of the invention, wherein the synthesized polynucleotides were amplified using the single primer amplification methods described in Kurn, U.S. Patent Publication No. 2003/0087251 A1, and the UNG treatment and amine fragmentation steps were performed in the same reaction mixture.

FIG. 8: shows a graph depicting the correlation observed between two populations of labeled fragments prepared from two independent RiboSPIATM amplification reactions using the single primer amplification methods described in Kurn, U.S. Patent Publication No. 2003/0087251 A1. Each sample was hybridized to two identical arrays, and intensities observed for each spot on the arrays are plotted against each other. The Pearson correlation coefficient was calculated, and a statistically significant correlation between duplicate arrays was observed (correlation coefficient r=0.98).

### MODES FOR CARRYING OUT THE INVENTION

Methods of the Invention

Methods for Labeling and Fragmenting a Polynucleotide, and Methods for Labeling a Polynucleotide

The invention provides novel methods and kits for labeling and fragmenting a polynucleotide, and novel methods and kits for labeling a polynucleotide. These methods are suitable for, for example, generation of labeled polynucleotides, or labeled polynucleotide fragments, for use as hybridization probes. Generally, the polynucleotide is labeled at an abasic site present in the polynucleotide, and fragmented at an abasic site present in the polynucleotide (in embodiments involving fragmentation). The abasic site present in the polynucleotide is generally prepared by cleavage of a base portion of a non-canonical nucleotide present in the polynucleotide. Thus, the spacing of the non-canonical nucleotide in the polynucleotide to be labeled and fragmented (in embodiments involving fragmentation), relates to and determines the size of fragments and intensity of labeling. This feature permits control of fragment size and/or site of labeling by use of conditions permitting controlled incorporation of non-canonical nucleotide, for example, during synthesis of the polynucleotide comprising the non-canonical nucleotide from a polynucleotide template.

Thus, in one aspect, the invention provides methods for labeling and fragmenting a polynucleotide. The methods generally comprise generation of a polynucleotide comprising a non-canonical nucleotide, cleavage of a base portion of the non-canonical nucleotide present in the polynucleotide with an agent (such as an enzyme) capable of cleaving a base portion of the non-canonical nucleotide (whereby an abasic site is generated); cleavage of the phosphodiester backbone at the abasic site, and labeling at the abasic site, whereby labeled polynucleotide fragments are generated. In another aspect, the invention provides methods for labeling a polynucleotide. The methods generally comprise generation of a polynucleotide comprising a non-canonical nucleotide, cleavage of a base portion of the non-canonical nucleotide present in the polynucleotide with an agent capable of cleaving a base portion of the non-canonical nucleotide (whereby an abasic site is generated); and labeling at the site of incorporation of the non-canonical nucleotide (i.e., at the abasic site), whereby a labeled polynucleotide(s) is generated.

The methods of labeling and fragmenting a polynucleotide and the methods of labeling a polynucleotide generally comprise synthesis of the polynucleotide comprising a non-canonical nucleotide from a polynucleotide template in the presence of a non-canonical nucleotide, whereby a polynucleotide comprising a non-canonical nucleotide(s) is generated.

Non-canonical nucleotides are known in the art and any suitable non-canonical polynucleotide can be used. In some embodiments, two or more different non-canonical nucleotides are used, such that a polynucleotide comprising two or more non-canonical nucleotides is generated. Method for synthesizing polynucleotides from a polynucleotide template are known in the art and described herein, and any suitable method can be used in the methods of the invention. In some embodiments, synthesis of the polynucleotide comprising the non-canonical nucleotides is using single primer isothermal amplification (see Kurn, U.S. Pat. No. 6,251,639 B1), Ribo-SPIA™ (see Kurn, U.S. Patent Publication No. 2003/0087251 A1), PCR, primer extension, reverse transcription, strand displacement amplification (SDA), multiple displacement amplification (MDA), DNA replication, and the like. The polynucleotide that is synthesized can single stranded, double-stranded or partially double stranded, and either or both strands can comprise a non-canonical nucleotide. In some embodiments, the polynucleotide that is synthesized comprises a cDNA. The polynucleotide template (along which the polynucleotide comprising a non-canonical nucleotide is synthesized) is any template from which labeled polynucleotide or fragments thereof is desired to be produced. In some embodiments, the template comprises RNA, mRNA, genomic DNA, cDNA, or synthetic DNA. In other embodiments, the template comprises a cDNA library, a subtractive hybridization library, or a genomic library. Generally, the polynucleotide comprising the non-canonical nucleotide is synthesized using limited and/or controlled incorporation of the non-canonical nucleotide, which results in generation of a polynucleotide with a frequency or proportion of non-canonical nucleotides such that, in embodiments involving fragmentation, labeled fragments of a desired size (or size range) are generated (following production of an abasic site, labeling at an abasic site, and cleavage of the phosphodiester backbone at an abasic site (in embodiments involving fragmentation). Similarly, in embodiments involving labeling but not fragmentation, labeled polynucleotides are produced (following production of an abasic site, and labeling at an abasic site).

In some embodiment, a labeled primer is used during synthesis of the polynucleotide comprising a non-canonical nucleotide. In other embodiments, a primer comprising a non-canonical nucleotide (such as dUTP) is used during synthesis of the polynucleotide comprising a non-canonical nucleotide. In other embodiments, the primer is a composite primer, said composite primer comprising a RNA portion and a 3′ DNA portion.

It is understood that a polynucleotide comprising a non-canonical nucleotide can be a multiplicity (from small to very large) of different polynucleotide molecules. Such populations can be related in sequence (e.g., members of a gene family or superfamily) or extremely diverse in sequence (e.g., generated from all mRNA, generated from all genomic DNA, etc.). Polynucleotides can also correspond to single sequences (which can be part or all of a known gene, for example a coding region, genomic portion, etc.).

A base portion of the non-canonical nucleotide is cleaved by an agent (such as an enzyme) capable of cleaving a base portion of a non-canonical nucleotide. Such agents are known in the art and described herein. In one embodiment, the agent capable of specifically cleaving a base portion of a non-canonical nucleotide is N-glycosylase. In another embodiment, the agent is Uracil N-Glycosylase (interchangeably termed “UNG” or “uracil DNA glyosylase”).

The polynucleotide comprising an abasic site is labeled using an agent capable of labeling an abasic site, and, in embodiments involving fragmentation, the phosphodiester backbone of the polynucleotide comprising an abasic site is cleaved at the site of incorporation of the non-canonical nucleotide (i.e., the abasic site by an agent capable of cleaving the phosphodiester backbone at an abasic site, such that two or more fragments are produced. As used herein, “cleaving the backbone or phosphodiester backbone” is also termed “fragmentation” or fragmenting”. In embodiments involving fragmentation, labeling can occur before fragmentation, fragmentation can occur before labeling, or fragmentation and labeling can occur simultaneously. For convenience, these steps are described separately below.

Agents capable of labeling (generally specifically labeling) an abasic site, whereby a polynucleotide (or polynucleotide fragment) comprising a labeled abasic site is generated, are known in the art. In some embodiments, the detectable moiety (label) is covalently or non-covalently associated with an abasic site. In some embodiments, the detectable moiety is directly or indirectly associated with an abasic site. In some embodiments, the detectable moiety (label) is directly or indirectly detectable. In some embodiments, the detectable signal is amplified. In some embodiments, the detectable moiety comprises an organic molecule. In other embodiments, the detectable moiety comprises an antibody. In other embodiments, the detectable signal is fluorescent. In other embodiments, the detectable signal is enzymatically generated. In some embodiments, the label is selected from 5-(((2-(carbohydrazino)-methyl)thio)acetyl)aminofluorescein, aminooxyacetyl hydrazide (“FARP”), N-(aminooxyacetyl)-N′-(D-biotinoyl) hydrazine, trifluoroacetic acid salt (ARP), Alexa Fluor 555, or an aminooxy-derivatized Alexa Fluor 555 (as described herein).

In embodiments involving fragmentation, the backbone of the polynucleotide comprising the abasic site is cleaved at the abasic site, whereby two or more fragments of the polynucleotide are generated. At least one of the fragments comprises an abasic site, which may be labeled and/or immobilized as described herein. Agents that cleave the phosphodiester backbone of a polynucleotide at an abasic site are known in the art. In some embodiments, the agent is E. coli AP endonuclease IV. In other embodiments, the agent is N,N′-dimethylethylenediamine (termed “DMED”). In other embodiments, the agent is heat, basic condition, acidic conditions, or an alkylating agent. Depending on the agent, the backbone can be cleaved 5′ to the abasic site (e.g., cleavage between the 5′-phosphate group of the abasic residue and the deoxyribose ring of the adjacent nucleotide, generating a free 3′ hydroxyl group), such that an abasic site is located at the 5′ end of the resulting fragment. In other embodiments, cleavage can also be 3′ to the abasic site (e.g., cleavage between the deoxyribose ring and 3′-phosphate group of the abasic residue and the deoxyribose ring of the adjacent nucleotide, generating a free 5′ phosphate group on the deoxyribose ring of the adjacent nucleotide), such that an abasic site is located at the 3′ end of the resulting fragment. In still other embodiments, more complex forms of cleavage are possible, for example, cleavage such that cleavage of the phosphodiester backbone and cleavage of a portion of the abasic nucleotide results. Selection of the fragmentation agent thus permits control of the orientation of the abasic site within the polynucleotide fragment, for example, at the 3′ end of the resulting fragment or the 5′ end of the resulting fragment. This feature has advantages, e.g., in embodiments involving immobilization as described below. Selection of reaction conditions also permits control of the degree, level or completeness of the fragmentation reactions. In some embodiments, reaction conditions can be selected such that the cleavage reaction is performed in the presence of a large excess of reagents and allowed to run to completion with minimal concern about excessive cleavage of the polynucleotide (i.e., while retaining a desired fragment size, which may be determined by spacing of the incorporated non-canonical nucleotide, during the synthesis step, above). By contrast, other methods known in the art, e.g., mechanical shearing, DNase cleavage, require careful titration of reaction conditions (including careful control of quantity of input DNA when DNase is used), to avoid excessive cleavage. In other embodiments, reaction conditions are selected such that fragmentation is not complete (in the sense that the backbone at some abasic sites remains uncleaved (unfragmented)), such that polynucleotide fragments comprising more than one abasic site are generated. Such fragments comprise internal (nonfragmented) abasic sites.

The methods of the invention include methods using the labeled polynucleotide fragments and labeled polynucleotides produced by the methods of the invention (so-called “applications”). The invention provides methods to characterize (for example, detect presence or absence of and/or quantify) a sequence of interest by analyzing the labeled and/or fragmented products by detection/quantification methods such as those based on array technologies or solution phase technologies. In some embodiments, the invention provides methods of detecting the presence or absence of mutations.

In other embodiments, the invention provides methods of producing a hybridization probe, hybridization using the hybridization probes; detection using the hybridization probes; characterizing and/or quantitating nucleic acid, preparing a subtractive hybridization probe, comparative genomic hybridization, and determining a gene expression profile, using the labeled and/or fragmented nucleic acids generated by the methods of the invention.

Methods for Immobilizing a Polynucleotide to a Substrate at an Abasic Site

The invention also provides methods for the generation of polynucleotides, or fragments thereof, immobilized to a substrate (surface). In some embodiments, the immobilized polynucleotide, or immobilized polynucleotide fragment (in embodiments involving fragmentation) is labeled according to the labeling methods described herein. These methods are suitable for, for example, the production of microarrays or tagged analytes.

As described herein, the abasic site is generally prepared by cleavage of a base portion of a non-canonical nucleotide present in the polynucleotide, and, as such, the spacing of the non-canonical nucleotide in the polynucleotide to be immobilized, optionally fragmented and/or optionally labeled, relates to and determines the site of immobilization, size of fragments (in embodiments involving fragmentation) and intensity of labeling (in embodiments involving labeling). This feature permits control of fragment size and/or intensity and location of labeling (in embodiments involving labeling) by use of conditions permitting controlled incorporation of non-canonical nucleotide, for example, during synthesis of the polynucleotide comprising the non-canonical nucleotide from a polynucleotide template.

Thus, in one aspect, the invention provides methods for immobilizing a polynucleotide to a substrate comprising cleavage of a base portion of a non-canonical nucleotide present in a polynucleotide comprising a non-canonical nucleotide with an agent capable of cleaving a base portion of the non-canonical nucleotide (whereby an abasic site is created); optionally, cleaving the phosphodiester backbone of the polynucleotide at the abasic site, whereby fragments are generated; and immobilizing the polynucleotide, or fragments thereof (in embodiments involving fragmentation) on a substrate at the abasic site. Generally, the polynucleotide comprising a non-canonical nucleotide is prepared using any method known in the art and as described herein. Agents capable of cleaving a base portion of a non-canonical nucleotide and, in embodiments involving fragmentation, agents capable of cleaving a phosphodiester backbone at an abasic site, are as described herein.

Optionally, the polynucleotides, or fragments thereof, are labeled according to any of the labeling methods described herein. Thus, in some embodiments, the invention provides methods for generating labeled polynucleotides, or labeled polynucleotide fragments, that are immobilized to a substrate. In some embodiments, the polynucleotide, or polynucleotide fragments are labeled according to any of the labeling methods disclosed herein.

The polynucleotide (or fragment thereof) comprising an abasic site is immobilized to a substrate at the abasic site. The substrate can be a solid or semi-solid surface, e.g., a microarray. In other embodiments, the microarray comprises at least one polynucleotide (or fragment thereof) immobilized on a substrate fabricated from a material selected from the group consisting of paper, glass, ceramic, plastic, polypropylene, polystyrene, nylon, polyacrylamide, nitrocellulose, silicon, and optical fiber. In other embodiments, the polynucleotide (or fragment thereof) is immobilized on the substrate in a two-dimensional configuration or a three-dimensional configuration comprising pins, rods, fibers, tapes, threads, beads, particles, microtiter wells, capillaries, and cylinders. In other embodiments, polynucleotide (or fragment thereof in embodiments involving fragmentation) comprising an abasic site is immobilized to a substrate selected from the group consisting of one or more of: protein, polypeptide, peptide, nucleic acid, carbohydrates, cells, microorganisms and fragments and products thereof, an organic molecule, and an inorganic molecule. In still other embodiment, the substrate is selected from a polypeptide, an antibody, an organic molecule and an inorganic molecule.

Single stranded polynucleotides (including polynucleotide fragments) are particularly suitable for preparing microarrays comprising the single stranded polynucleotides. Single stranded polynucleotide fragments (in embodiments involving cleavage of the phosphodiester backbone at an abasic site) are advantageous, because the orientation of the fragment with respect to the surface (upon which the fragment is immobilized) can be controlled by selection of the method used to cleave the phosphodiester backbone, such that an abasic site is positioned at the 3′ end of a fragment or at the 5′ end of a fragment. Immobilizing polynucleotides in a defined orientation (e.g., at the 3′ end, at the 5′ end) enhances hybridization of complementary oligonucleotides, and permits a higher density of immobilization.

The methods of the invention include methods using the immobilized polynucleotides, or immobilized polynucleotide fragments produced by the methods of the invention (so-called “applications”). In some embodiments, the invention provides methods of detecting nucleic acid sequence mutations.

The invention also provides methods to characterize (for example, detect presence or absence of and/or quantify) a sequence of interest using the immobilized polynucleotides, or fragments thereof

In another embodiment, the invention provides methods of determining a gene expression profile, using the immobilized polynucleotides, or fragments thereof, generated by the methods of the invention.

General Techniques

The practice of the invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Primers, oligonucleotides and polynucleotides employed in the invention can be generated using standard techniques known in the art.

Definitions

A “template sequence,” or “template nucleic acid” or “template” as used herein, is a polynucleotide comprising a sequence of interest, for which synthesis of a complement comprising a non-canonical nucleotide is desired. The template sequence may be known or not known, in terms of its actual sequence. In some instances, the terms “target,” “template,” and variations thereof, are used interchangeably.

“Polynucleotide,” or “nucleic acid,” as used interchangeably herein, refer to polymers of nucleotides of any length, and include DNA. The nucleotides can be deoxyribonucleotides, modified nucleotides or bases, and/or their analogs, or any substrate that can be incorporated into a polymer by DNA polymerase. Nucleotides include canonical and non-canonical nucleotides and a polynucleotide can comprise canonical and non-canonical nucleotides. A polynucleotide may comprise modified (altered) nucleotides, such as, for example, modification to the nucleotide structure and or modification to the phosphodiester backbone. As discussed herein modified nucleotide can be canonical nucleotide or non-canonical (cleavable) nucleotides. It is understood, however, that modified nucleotides that are not non-canonical (cleavable) nucleotide under the reaction conditions used in the methods of the invention, if present, generally should not affect the ability of the polynucleotide to undergo cleavage of a base portion of non-canonical nucleotide, such that an abasic site is generated, and/or cleavage of a phosphodiester backbone at an abasic site, such that fragments are generated, and/or immobilization of a polynucleotide (or fragment thereof) to a substrate, as described herein. If present, modification to the nucleotide structure, such as methylated nucleotides may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). It is understood that internucleotide modifications may, e.g., alter the efficiency and/or kinetics of cleavage of the phosphodiester backbone (as when, for example a phosphodiester backbone is cleaved at an abasic site, as described herein). Further, any of the hydroxyl groups ordinarily present in the sugars may be replaced, for example, by phosphonate groups, phosphate groups, protected by standard protecting groups, or activated to prepare additional linkages to additional nucleotides. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′—O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including DNA. It is understood, however, that modified nucleotides and/or internucleotide linkages and/or, if present, generally should not affect the ability of the polynucleotide to undergo cleavage of a base portion of a non-canonical nucleotide, such that an abasic site is generated, and/or the ability of a polynucleotide to undergo cleavage of a phosphodiester backbone at an abasic site, such that fragments are generated, and/or the ability of a polynucleotide to be immobilized at an abasic site (such as an abasic site at an end of a polynucleotide and/or an abasic site that is not at an end of a polynucleotide) to a surface, as described herein.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

A “primer,” as used herein, refers to a nucleotide sequence (a polynucleotide), generally with a free 3′-OH group, that hybridizes with a template sequence (such as a template RNA, or a primer extension product) and is capable of promoting polymerization of a polynucleotide complementary to the template. A “primer” can be, for example, an oligonucleotide. It can also be, for example, a sequence of the template (such as a primer extension product or a fragment of an RNA template created following RNase cleavage of a template RNA-DNA complex) that is hybridized to a sequence in the template itself (for example, as a hairpin loop), and that is capable of promoting nucleotide polymerization. Thus, a primer can be an exogenous (e.g., added) primer or an endogenous (e.g., template fragment) primer.

A “complex” is an assembly of components. A complex may or may not be stable and may be directly or indirectly detected. For example, as is described herein, given certain components of a reaction, and the type of product(s) of the reaction, existence of a complex can be inferred. For purposes of this invention, a complex is generally an intermediate with respect to the final polynucleotide fragments, labeled polynucleotide, labeled polynucleotide fragments, and/or immobilized polynucleotide or fragment thereof.

A “fragment” of a polynucleotide or oligonucleotide is a contiguous sequence of 2 or more bases. In other embodiments, a fragment (also termed “region” or “portion”) is any of about 3, about 5, about 10, about 15, about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In some embodiments, the fragments can be at least about 3, about 5, about 10, about 15, about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In other embodiments, the fragments can be less than about 3, about 5, about 10, about 15, about 20, about 25, about 30 about 35 about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In some embodiment, these fragment lengths represent an average size in the population of fragments generated using the methods of the invention.

A “reaction mixture” is an assemblage of components, which, under suitable conditions, react to form a complex (which may be an intermediate) and/or a product(s).

“A”, “an” and “the”, and the like, unless otherwise indicated include plural forms. “A” fragment means one or more fragments. “A” non-canonical nucleotide means one or more non-canonical nucleotides.

“Comprising” means including in accordance with well-established principles of patent law.

Conditions that “allow” an event to occur or conditions that are “suitable” for an event to occur, such as polynucleotide synthesis, cleavage of a base portion of a non-canonical nucleotide, cleavage of a phosphodiester backbone at an abasic site, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the polynucleotide sequence, temperature, and buffer conditions. These conditions also depend on what event is desired, such as polynucleotide synthesis, cleavage of a base portion of a non-canonical nucleotide, cleavage of a phosphodiester backbone at an abasic site, labeling an abasic site, immobilizing a polynucleotide fragment or a polynucleotide, etc.

“Microarray” and “array,” as used interchangeably herein, comprise a surface with an array, preferably ordered array, of putative binding (e.g., by hybridization) sites for a biochemical sample (target) which often has undetermined characteristics. In a preferred embodiment, a microarray refers to an assembly of distinct polynucleotide or oligonucleotide probes immobilized at defined positions on a substrate. Arrays are formed on substrates fabricated with materials such as paper, glass, plastic (e.g., polypropylene, nylon, polystyrene), polyacrylamide, nitrocellulose, silicon and other metals, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al., Nature Genetics (1996), 14:457-460; Shalon et al., Genome Res. (1996), 6:639-645; and Schena et al., Proc. Natl. Acad. Sci. U.S.A. (1995), 93:10539-11286); (iii) by masking (Maskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)). Probes may also be noncovalently immobilized on the substrate by hybridization to anchors, by means of magnetic beads, or in a fluid phase such as in microtiter wells or capillaries. The probe molecules are generally nucleic acids such as DNA, RNA, PNA, and cDNA but may also include proteins, polypeptides, oligosaccharides, cells, tissues and any permutations thereof which can specifically bind the target molecules.

The term “3′” generally refers to a region or position in a polynucleotide or oligonucleotide 3′ (downstream) from another region or position in the same polynucleotide or oligonucleotide.

The term “5′” generally refers to a region or position in a polynucleotide or oligonucleotide 5′ (upstream) from another region or position in the same polynucleotide or oligonucleotide.

The term “3′-DNA portion,” “3′-DNA region,” “3′-RNA portion,” and “3′-RNA region,” refer to the portion or region of a polynucleotide or oligonucleotide located towards the 3′ end of the polynucleotide or oligonucleotide, and may or may not include the 3′ most nucleotide(s) or moieties attached to the 3′ most nucleotide of the same polynucleotide or oligonucleotide. The 3′ most nucleotide(s) can be preferably from about 1 to about 50, more preferably from about 10 to about 40, even more preferably from about 20 to about 30 nucleotides.

As used herein, “canonical” nucleotide means a nucleotide comprising one the four common nucleic acid bases adenine, cytosine, guanine and thymine that are commonly found in DNA. The term also encompasses the respective deoxyribonucleosides, deoxyribonucleotides or 2′-deoxyribonucleoside-5′-triphosphates that contain one of the four common nucleic acid bases adenine, cytosine, guanine and thymine (though as explained herein, the base can be a modified and/or altered base as discussed, for example, in the definition of polynucleotide). As used herein, the base portions of canonical nucleotides are generally not cleavable under the conditions used in the methods of the invention.

As used herein, “non-canonical nucleotide” (interchangeably called “non-canonical deoxyribonucleoside triphosphate”) refers to a nucleotide comprising a base other than the four canonical bases. The term also encompasses the respective deoxyribonucleosides, deoxyribonucleotides or 2′-deoxyribonucleoside-5′-triphosphates that contain a base other than the four canonical bases. In the context of this invention, nucleotides containing uracil (such as dUTP), or the respective deoxyribonucleosides, deoxyribonucleotides or 2′-deoxyribonucleoside-5′-triphosphates, are a non-canonical nucleotides. As used herein, the base portions of non-canonical nucleotides are capable of being, generally, specifically or selectively cleaved (such that a nucleotide comprising an abasic site is created) under the reaction conditions used in the methods of the invention. As described herein, non-canonical nucleotides are generally also capable of being incorporated into a polynucleotide during synthesis of a polynucleotide (during e.g., primer extension and/or replication); capable of being generally, specifically or selectively cleaved by an agent that cleaves a base portion of a nucleotide, such that a polynucleotide comprising an abasic site is generated; comprise a suitable internucleotide connection (when incorporated into a polynucleotide) such that a phosphodiester backbone at an abasic site (i.e., the non-canonical nucleotide following cleavage of a base portion) is capable of being cleaved by an agent capable of such cleavage; capable of being labeled (following generation of an abasic site); and/or capable of immobilization to a surface (following generation of an abasic site), according to the methods described herein. It is understood that the non-canonical nucleotide may, but does not necessarily, require all of the features described above, depending on the particular method of the invention in which the non-canonical nucleotide is to be used. In some embodiments, non-canonical nucleotides are altered and/or modified nucleotides as described herein. Non-canonical nucleotide refers to a nucleotide that is incorporated into a polynucleotide as well as to a single nucleotide.

The term “analyte” as used herein refers to a substance to be detected or assayed by the method of the present invention, for example, a compound whose properties, location, quantity and/or identity is desired to be characterized. Typical analytes may include, but are not limited to proteins, peptides, nucleic acid segments, cells, microorganisms and fragments and products thereof, organic molecules, inorganic molecules, or any substance for which immobilization sites for binding partner(s) can be developed. As this disclosure clearly conveys, an analyte is a substrate.

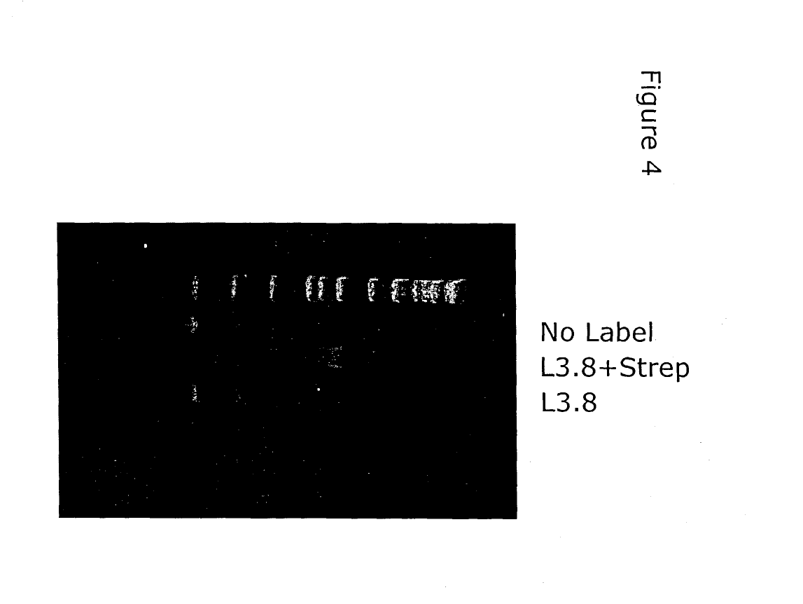

As used herein, an “abasic site” refers to the site of incorporation of the non-canonical nucleotide following treatment with an agent capable of effecting cleavage of a base portion of the non-canonical nucleotide. An abasic site (interchangeably termed “AP site”) can comprise a hemiacetal ring, and lacks a base portion of the non-canonical nucleotide. As used herein, “abasic site” encompasses any chemical structure remaining following treatment of a non-canonical nucleotide (present in a polynucleotide chain) with an agent (e.g., an enzyme, or heat or basic conditions) capable of effecting cleavage of a base portion of a non-canonical nucleotide. Thus, an abasic site as used herein includes a modified sugar moiety attached to the 3′ terminus of nicked polynucleotide, as when, for example, endonuclease III or OGGI protein are used to cleave the base portion of the non-canonical nucleotide. See, e.g., Kow, (2000) Methods 22, 164-169 (e.g., FIG. 4).

As used herein, “labeling at an abasic site” means association of a label with any chemical structure remaining following removal of a base portion (including the entire base) of a non-canonical nucleotide (present in a polynucleotide chain) by treatment with an agent (e.g., an enzyme, or heat)) capable of effecting cleavage of a base portion of a non-canonical nucleotide. In one embodiment, a reactive aldehyde form of a hemiacetal ring in an abasic site is labeled. In other embodiments, the label associate with a chemical structure remaining following treatment of a non-canonical nucleotide (present in a polynucleotide chain) with an agent (e.g., an enzyme, or heat or basic conditions) capable of effecting cleavage of a base portion of a non-canonical nucleotide and treatment of polynucleotide comprising an abasic site with an agent capable of effecting cleavage of the backbone at the abasic site (as described herein).

As used herein, cleavage of a backbone (e.g. phosphodiester backbone) “at” an abasic site means cleavage of the phosphodiester linkage 3′ to the abasic site or 5′ to the abasic site, or both. As the disclosure herein conveys, “at” an abasic site refers to proximate or near location (such as immediately 3′ or immediately 5′). In still other embodiments, more complex forms of cleavage are possible, for example, cleavage such that cleavage of the phosphodiester backbone and cleavage of (a portion of) the abasic nucleotide results.

As used herein, a “label” (interchangeably called a “detectable moiety”) refers to a moiety that is associated or linked with a polynucleotide (interchangeably called “labeling”). The labeled polynucleotide may be directly or indirectly detected, generally through a detectable signal. The detectable moiety (label) can be attached (or associated) either directly or through a non-interfering linkage group with other moieties capable of specifically associating with one or more sites to be labeled. The detectable moiety (label) may be covalently or non-covalently associated as well as directly or indirectly associated.

The following are examples of the methods of the invention. It is understood that various other embodiments may be practiced, given the general description provided herein. For example, reference to using an agent capable of cleaving a base portion of the non-canonical nucleotide means that any of the agents capable of cleaving a base portion of the non-canonical nucleotide described herein may be used.

Methods for Labeling and Fragmenting Nucleic Acids

The invention provides methods for generating labeled fragments, of nucleic acid. The methods generally comprise generation of a polynucleotide comprising at least one non-canonical nucleotide, cleavage of a base portion of the non-canonical nucleotide present in the polynucleotide with an agent capable of cleaving a base portion of the non-canonical nucleotide; and cleavage of the phosphodiester backbone of the polynucleotide comprising the abasic site at the abasic site; and labeling at the abasic site, whereby labeled nucleic acid fragments are generated. Generally, the polynucleotide comprising a non-canonical nucleotide is fragmented and labeled at the site of incorporation of the non-canonical nucleotide(s) present in the synthesized polynucleotide. Thus, the frequency of non-canonical nucleotides in the synthesized polynucleotide generally relates to and determines the size range of the labeled fragments produced from the polynucleotide. The methods of the invention generate labeled nucleic acid fragments, which are useful for, for example, hybridization to a microarray and other uses described herein.

For convenience, the synthesis of a polynucleotide comprising a non-canonical nucleotide, and the treatment of that polynucleotide with an agent, such as an enzyme, capable of cleaving a base portion of the non-canonical nucleotide are described as separate steps. It is understood that these steps (e.g., one or more of these steps) may be performed simultaneously, except (generally) in the case when a polynucleotide comprising a non-canonical nucleotide must be capable of serving as a template for further amplification (as in exponential methods of amplification, e.g. PCR), in which case it is preferable to synthesize the polynucleotide comprising an abasic site prior to cleaving the base portion of the non-canonical nucleotide.

The methods involve the following steps: (a) synthesizing a polynucleotide from a template in the presence of a non-canonical nucleotide (interchangeably termed “non-canonical deoxyribonucleoside triphosphate” or “non-canonical deoxyribonucleotide”), whereby a polynucleotide comprising a non-canonical nucleotide is generated; (b) contacting the polynucleotide comprising a non-canonical nucleotide with an agent capable of cleaving a base portion of the non-canonical nucleotide (i.e., cleaving a base portion of the non-canonical nucleotide), whereby an abasic site is created; (c) cleaving the backbone of the polynucleotide comprising the abasic site at the abasic site; and (d) contacting the polynucleotide comprising the abasic site with an agent capable of labeling the abasic site (i.e., labeling the abasic site), whereby labeled polynucleotide fragments are generated.

For simplicity, individual steps of the labeling and fragmentation method are discussed below. It is understood, however, that the steps may be performed simultaneously and/or in varied order, as discussed herein.

Synthesis of a Polynucleotide Comprising a Non-Canonical Nucleotide

The methods involve synthesizing a polynucleotide from a template in the presence of at least one non-canonical nucleotide (interchangeably termed “non-canonical deoxyribonucleoside triphosphate”), whereby a polynucleotide comprising a non-canonical nucleotide is generated. The frequency of incorporation of non-canonical nucleotides into the polynucleotide relates to the size of fragment produced using the methods of the invention because the spacing between non-canonical nucleotides in the polynucleotide comprising a non-canonical nucleotide, along with the reaction conditions used, determines the approximate size of the fragments resulting from generation of an abasic site from the non-canonical nucleotide and cleavage of the backbone at the abasic site, as described herein.

Generally, the polynucleotide is DNA, though, as noted herein, the polynucleotide can comprise altered and/or modified nucleotides, internucleotide linkages, ribonucleotides, etc. As generally used herein, it is understood that “DNA” applies to polynucleotide embodiments.

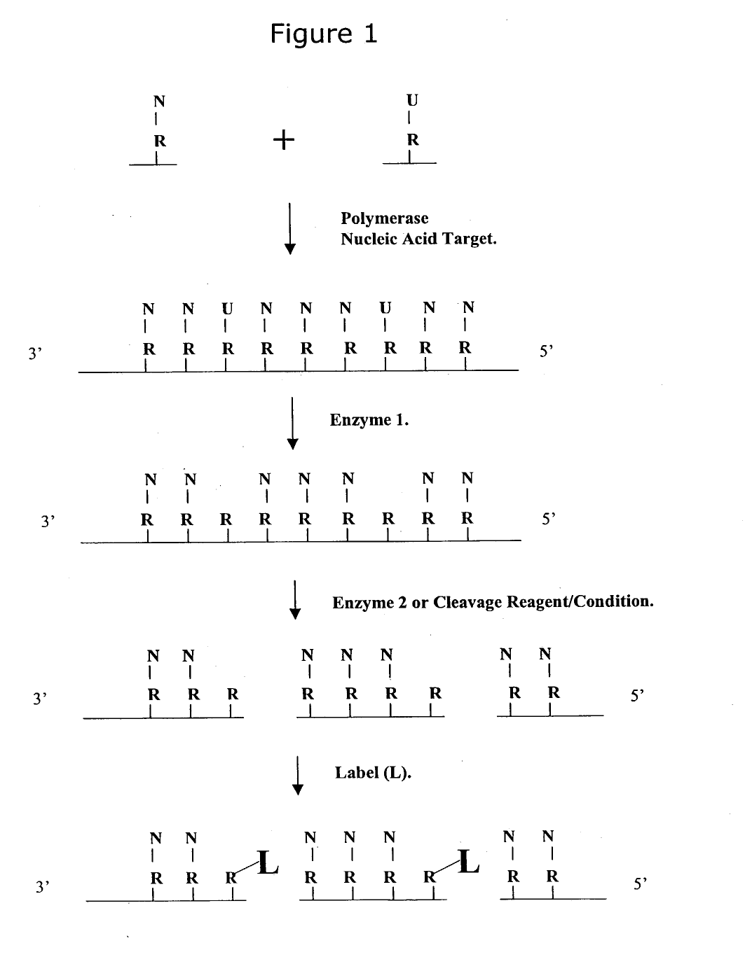

Methods for synthesizing polynucleotides, e.g., single and double stranded DNA, from a template are well known in the art, and include, for example, single primer isothermal amplification, Ribo-SPIA™, PCR, reverse transcription, primer extension, limited primer extension, replication (including rolling circle replication), strand displacement amplification (SDA), nick translation, multiple displacement amplification (MDA), and, e.g., any method that results in synthesis of the complement of a template sequence such that at least one non-canonical nucleotide can be incorporated into a polynucleotide. See, e.g., Kurn, U.S. Pat. No. 6,251,639 B1; Kurn, WO 02/00938; Kurn, U.S. Patent Publication No. 2003/0087251 A1; Mullis, U.S. Pat. No. 4,582,877; Wallace, U.S. Pat. No. 6,027,923; U.S. Pat. Nos. 5,508,178; 5,888,819; 6,004,744; 5,882,867; 5,710,028; 6,027,889; 6,004,745; 5,763,178; 5,011,769; see also Sambrook (1989) “Molecular Cloning: A Laboratory Manual”, second edition; Ausebel (1987, and updates) “Current Protocols in Molecular Biology”; Mullis, (1994) “PCR: The Polymerase Chain Reaction”. One or more methods known in the art can be used to generate a polynucleotide comprising a non-canonical nucleotide. It is understood that the polynucleotide comprising a non-canonical nucleotide can be single stranded or double stranded or partially double stranded, and that one or both strands of a double stranded polynucleotide can comprise a non-canonical nucleotide. For convenience, “DNA” is used herein to describe (and exemplify) a polynucleotide. Suitable methods include methods that result in one single- or double-stranded polynucleotide comprising a non-canonical nucleotide (for example, reverse transcription, production of double stranded cDNA, a single round of DNA replication), as well as methods that result in multiple single stranded or double stranded copies or copies of the complement of a template (for example, single primer isothermal amplification or Ribo-SPIA™ or PCR). In one embodiment, illustrated in FIG. 1, a single-stranded polynucleotide comprising a non-canonical nucleotide is synthesized using single primer isothermal amplification. See Kurn, U.S. Pat. No. 6,251,639 B1.

Generally, the polynucleotide comprising a non-canonical nucleotide is generated from a template in-the presence of all four canonical nucleotides and at least one non-canonical nucleotide under reaction conditions suitable for synthesis of polynucleotides, including suitable enzymes and primers, if necessary. Reaction conditions and reagents, including primers, for synthesizing the polynucleotide comprising a non-canonical nucleotide are known in the art, and further discussed herein. As described herein, non-canonical nucleotides are generally capable of polymerization (i.e., are substrates for DNA polymerase), and capable of being rendered abasic following treatment with a suitable agent capable of generally, specifically or selectively cleaving a base portion of a non-canonical nucleotide. Suitable non-canonical nucleotides are well-known in the art, and include: deoxyuridine triphosphate (dUTP), deoxyinosine triphosphate (dITP), 5-hydroxymethyl deoxycytidine triphosphate (5-OH-Me-dCTP). See, e.g., Jendrisak, U.S. Pat. No. 6,190,865 B1; Mol. Cell Probes (1992) 251-6. Generally, in embodiments in which a polynucleotide comprising an non-canonical nucleotide serves as a template for further amplification (e.g., as when multiple copies of a double stranded polynucleotides comprising a non-canonical nucleotide are synthesized, e.g., by PCR amplification), a polynucleotide comprising a non-canonical nucleotide must be capable of serving as a template for further amplification.

It is understood that two or more different non-canonical nucleotides can be incorporated into the polynucleotide synthesized from the template by DNA polymerase, whereby a polynucleotide comprising at least two different non-canonical nucleotides is generated.

Conditions for limited and/or controlled incorporation of a non-canonical nucleotide are known in the art. See, e.g., Jendrisak, U.S. Pat. No. 6,190,865 B1; Mol. Cell Probes (1992) 251-6; Anal. Biochem. (1993) 211:164-9; see also Sambrook (1989) “Molecular Cloning: A Laboratory Manual”, second edition; Ausebel (1987, and updates) “Current Protocols in Molecular Biology”. The frequency (or spacing) of non-canonical nucleotides in the resulting polynucleotide comprising a non-canonical nucleotide, and thus the average size of fragments generated using the methods of the invention (i.e., following cleavage of a base portion of a non-canonical nucleotide, and cleavage of a phosphodiester backbone at a non-canonical nucleotide), is controlled by variables known in the art, including: frequency of nucleotide(s) corresponding to the non-canonical nucleotide(s) in the template (or other measures of nucleotide content of a sequence, such as average G-C content), ratio of canonical to non-canonical nucleotide present in the reaction mixture; ability of the polymerase to incorporate the non-canonical nucleotide, relative efficiency of incorporation of non-canonical nucleotide verses canonical nucleotide, and the like. It is understood that the average fragmentation size also relates to the reaction conditions used during fragmentation, as is further discussed herein. The reaction conditions can be empirically determined, for example, by assessing average fragment size generated using the methods of the invention taught herein. The level of labeling at an abasic site also relates to the frequency of incorporation of non-canonical nucleotides, as is further discussed herein.

Generally, a non-canonical base can be incorporated at about every 5, 10, 15, 20, 25, 30, 40, 50, 65, 75, 85, 100, 123, 150, 175, 200, 225, 250, 300, 350, 400, 450, 500, 550, 600, 650 or more nucleotides apart in the resulting polynucleotide comprising a non-canonical nucleotide. In one embodiment, the non-canonical nucleotide is incorporated about every 200 nucleotides, about every 100 nucleotide, or about every 50 nucleotide. In another embodiment, the non-canonical nucleotide is incorporated about every 50 to about 200 nucleotides. In some embodiments, a 1:5 ratio of dUTP and dTTP is used in the reaction mixture.

The polynucleotide template (along which the polynucleotide comprising a non-canonical nucleotide is synthesized) may be any template from which labeled polynucleotide fragments are desired to be produced. As is evident from the description herein, the labeled polynucleotide fragments are the complement of the sequence of the polynucleotide template. The template includes double-stranded, partially double-stranded, and single-stranded nucleic acids from any source in purified or unpurified form, which can be DNA (dsDNA and ssDNA) or RNA, including tRNA, mRNA, rRNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and fragments thereof. Obtaining and purifying nucleic acids use standard techniques in the art. RNAs can be obtained and purified using standard techniques in the art. A DNA template (including genomic DNA template) can be transcribed into RNA form, which can be achieved using methods disclosed in Kurn, U.S. Pat. No. 6,251,639 B1, and by other techniques (such as expression systems) known in the art. RNA copies of genomic DNA would generally include untranscribed sequences generally not found in mRNA, such as introns, regulatory and control elements, etc. DNA copies of an RNA template can be synthesized using methods described in Kurn, U.S. Patent Publication No. 2003/0087251 A1 or other techniques known in the art). Synthesis of polynucleotide comprising a non-canonical nucleotide from a DNA-RNA hybrid can be accomplished by denaturation of the hybrid to obtain a ssDNA and/or RNA, cleavage with an agent capable of cleaving RNA from an RNA/DNA hybrid, and other methods known in the art. The template can be only a minor fraction of a complex mixture such as a biological sample and can be obtained from various biological material by procedures well known in the art. The template can be known or unknown and may contain more than one desired specific nucleic acid sequence of interest, each of which may be the same or different from each other. Therefore, the methods of the invention are useful not only for producing one specific polynucleotide comprising a non-canonical nucleotide, but also for producing simultaneously more than one different specific polynucleotides comprising a non-canonical nucleotide. The template DNA can be a sub-population of nucleic acids, for example, a subtractive hybridization probe, total genomic DNA, restriction fragments, a cDNA library, cDNA prepared from total mRNA, a cloned library, or amplification products of any of the templates described herein. In some cases, the initial step of the synthesis of the complement of a portion of a template nucleic acid sequence is template denaturation. The denaturation step may be thermal denaturation or any other method known in the art, such as alkali treatment.

For simplicity, the polynucleotide comprising a non-canonical nucleotide is described as a single nucleic acid. It is understood that the polynucleotide can be a single polynucleotide, or a population of polynucleotides (from a few to a multiplicity to a very large multiplicity of polynucleotides). It is further understood that a polynucleotide comprising a non-canonical nucleotide can be a multiplicity (from small to very large) of different polynucleotide molecules. Such populations can be related in sequence (e.g., member of a gene family or superfamily) or extremely diverse in sequence (e.g., generated from all mRNA, generated from all genomic DNA, etc.). Polynucleotides can also correspond to single sequence (which can be part or all of a known gene, for example a coding region, genomic portion, etc.). Methods, reagents, and reaction conditions for generating specific polynucleotide sequences and multiplicities of polynucleotide sequences are known in the art.

Suitable methods of synthesis of a polynucleotide comprising a non-canonical nucleotide are generally template-dependent (in the sense that polynucleotide comprising a non-canonical nucleotide is synthesized along a polynucleotide template, as generally described herein). It is understood that non-canonical nucleotides can be incorporated into a polynucleotide as a result of template-independent methods. For example, one or more primer(s) can be designed to comprise one or more non-canonical nucleotides. See, e.g., Richards, U.S. Pat. Nos. 6,037,152, 5,427,929, and 5,876,976. As discussed herein, inclusion of at least one non-canonical nucleotide in a primer results in cleavage of a base-portion of a non-canonical nucleotide and labeling at the abasic site (i.e., following generation of an abasic site, as described herein), thus generating a polynucleotide fragment or a labeled polynucleotide fragment comprising a portion of the primer. Inclusion of a non-canonical nucleotide in a primer may be particularly suitable for methods such as single primer isothermal amplification. See Kurn, U.S. Pat. No. 6,251,639 B 1; Kurn, WO 02/00938; Kurn, U.S. Patent Publication No. 2003/0087251 Al. Non-canonical nucleotide(s) can also be added to a polynucleotide by template-independent methods such as tailing and ligation of a second polynucleotide comprising a non-canonical nucleotide. Methods for tailing and ligation are well-known in the art.

Cleaving a Base Portion of the Non-Canonical Nucleotide to Create an Abasic Site

The polynucleotide comprising a non-canonical nucleotide is treated with an agent, such as an enzyme, capable of generally, specifically, or selectively cleaving a base portion of the non-canonical deoxyribonucleoside to create an abasic site. The exemplary embodiment shown in FIG. 1 illustrates cleavage of a base portion of the non-canonical nucleotides, by an enzyme, whereby an abasic site is created. As used herein, “abasic site” encompasses any chemical structure remaining following removal of a base portion (including the entire base) with an agent capable of cleaving a base portion of a nucleotide, e.g., by treatment of a non-canonical nucleotide (present in a polynucleotide chain) with an agent (e.g., an enzyme, acidic conditions, or a chemical reagent) capable of effecting cleavage of a base portion of a non-canonical nucleotide. In some embodiments, the agent (such as an enzyme) catalyzes hydrolysis of the bond between the base portion of the non-canonical nucleotide and a sugar in the non-canonical nucleotide to generate an abasic site comprising a hemiacetal ring and lacking the base (interchangeably called “AP” site), though other cleavage products are contemplated for use in the methods of the invention. Suitable agents and reaction conditions for cleavage of base portions of non-canonical nucleotides are known in the art, and include: N-glycosylases (also called “DNA glycosylases” or “glycosidases”) including Uracil N-Glycosylase (“UNG”; specifically cleaves dUTP) (interchangeably termed “uracil DNA glyosylase”), hypoxanthine-N-Glycosylase, and hydroxy-methyl cytosine-N-glycosylase; 3-methyladenine DNA glycosylase, 3- or 7-methylguanine DNA glycosylase, hydroxymethyluracile DNA glycosylase; T4 endonuclease V. See, e.g., Lindahl, PNAS (1974) 71(9):3649-3653; Jendrisak, U.S. Pat. No. 6,190,865 B1. In one embodiment, uracil-N-glycosylase is used to cleave a base portion of the non-canonical nucleotide. In other embodiments, the agent that cleaves the base portion of the non-canonical nucleotide is the same agent that cleaves a phosphodiester backbone at the abasic site.

Generally, cleavage of base portions of non-canonical nucleotides is general, specific or selective cleavage (in the sense that the agent (such as an enzyme) capable of cleaving a base portion of a non-canonical nucleotide generally, specifically or selectively cleaves the base portion of a particular non-canonical nucleotide), whereby greater than about 98%, about 95%, about 90%, about 85%, or about 80% of the base portions cleaved are base portions of non-canonical nucleotides. However, extent of cleavage can be less. Thus, reference to specific cleavage is exemplary. General, specific or selective cleavage is desirable for control of the fragment size in the methods of generating labeled polynucleotide fragments of the invention (i.e., the fragments generated by cleavage of the backbone at an abasic site). Generally, reaction conditions are selected such that the reaction in which the abasic site(s) are created can run to completion.

In some embodiments, the polynucleotide comprising a non-canonical nucleotide is purified following synthesis of the non-canonical polynucleotide (to eliminate, for example, residual free non-canonical nucleotides that are present in the reaction mixture). In other embodiments (such as the embodiment described in Example 4), there is no intermediate purification between the synthesis of the polynucleotide comprising the non-canonical nucleotide and subsequent steps (such as cleavage of a base portion of the non-canonical nucleotide and cleavage of a phosphodiester backbone at the abasic site).

As noted herein, for convenience, cleavage of a base portion of a non-canonical nucleotide (whereby an abasic site is generated) has been described as a separate step. It is understood that this step may be performed simultaneously with synthesis of the polynucleotide comprising a non-canonical nucleotide (as described above), cleavage of the backbone at an abasic site (fragmentation) and/or labeling at an abasic site.

It is understood that the choice of non-canonical nucleotide can dictate the choice of enzyme to be used to cleave the base portion of that non-canonical enzyme, to the extent that particular non-canonical nucleotides are recognized by particular enzymes that are capable of cleaving a base portion of the non-canonical nucleotide.

Cleaving the Backbone at the Abasic Site of the Polynucleotide comprising an Abasic Site and Labeling at the Abasic Site

The backbone of the polynucleotide is cleaved at the abasic site, and the abasic site is labeled, whereby labeled fragments of nucleotide are generated. It is understood that cleavage of the backbone and labeling can be performed in any order, or simultaneously. For convenience, however, these reactions are described as separate steps.

Cleaving the Backbone at the Abasic Site of the Polynucleotide Comprising an Abasic Site

Following generation of an abasic site by cleavage of the base portion of the non-canonical nucleotide present in the polynucleotide, the backbone of the polynucleotide is cleaved at the site of incorporation of the non-canonical nucleotide (also termed the abasic site, following cleavage of the base portion of the non-canonical nucleotide) with an agent capable of effecting cleavage of the backbone at the abasic site. Cleavage at the backbone (also termed “fragmentation”) results in at least two fragments (depending on the number of abasic sites present in the polynucleotide comprising an abasic site, and the extent of cleavage).

Suitable agents (for example, an enzyme, a chemical and/or reaction conditions such as heat) capable of cleavage of the backbone at an abasic site are well known in the art, and include: heat treatment and/or chemical treatment (including basic conditions, acidic conditions, alkylating conditions, or amine mediated cleavage of abasic sites, (see e.g., McHugh and Knowland, Nucl. Acids Res. (1995) 23(10):1664-1670; Bioorgan. Med. Chem (1991) 7:2351; Sugiyama, Chem. Res. Toxicol. (1994) 7: 673-83; Horn, Nucl. Acids. Res., (1988) 16:11559-71), and use of enzymes that catalyze cleavage of polynucleotides at abasic sites, for example AP endonucleases (also called “apurinic, apyrimidinic endonucleases”) (e.g., E. coli Endonuclease IV, available from Epicentre Tech., Inc, Madison Wis.), E. coli endonuclease III or endonuclease IV, E. coli exonuclease III in the presence of calcium ions. See, e.g. Lindahl, PNAS (1974) 71(9):3649-3653; Jendrisak, U.S. Pat. No. 6,190,865 B1; Shida, Nucleic Acids Res. (1996) 24(22):4572-76; Srivastava, J. Biol Chem. (1998) 273(13):21203-209; Carey, Biochem. (1999) 38:16553-60; Chem Res Toxicol (1994) 7:673-683. As used herein “agent” encompasses reaction conditions such as heat. In one embodiment, the AP endonuclease, E. coli endonuclease IV, is used the cleave the phosphodiester backbone at an abasic site. In another embodiment, cleavage is with an amine, such as N, N′-dimethylethylenediamine. See, e.g. McHugh and Knowland, supra.

Generally, cleavage is between the nucleotide immediately 5′ to the abasic residue and the abasic residue, or between the nucleotide immediately 3′ to the abasic residue and the abasic residue (though, as explained herein, 5′ or 3′ cleavage of the phosphodiester backbone may or may not result in retention of the phosphate group 5′ or 3′ to the abasic site, respectively, depending on the fragmentation agent used). As is well known in the art, cleavage can be 5′ to the abasic site (such as endonuclease IV treatment which generally results in cleavage of the backbone at a location immediately 5′ to the abasic site between the 5′-phosphate group of the abasic residue and the deoxyribose ring of the adjacent nucleotide, generating a free 3′ hydroxyl group on the adjacent nucleotide), such that an abasic site is located at the 0.5′ end of the resulting fragment. Cleavage can also be 3′ to the abasic site (e.g., cleavage between the deoxyribose ring and 3′-phosphate group of the abasic residue and the deoxyribose ring of the adjacent nucleotide, generating a free 5′ phosphate group on the deoxyribose ring of the adjacent nucleotide), such that an abasic site is located at the 3′ end of the resulting fragment. Treatment under basic conditions or with amines (such as N,N′-dimethylethylenediamine) results in cleavage of the phosphodiester backbone immediately 3′ to the abasic site. In addition, more complex forms of cleavage are also possible, for example, cleavage such that cleavage of the phosphodiester backbone and cleavage of (a portion of) the abasic nucleotide results. For example, under certain conditions, cleavage using chemical treatment and/or thermal treatment may comprise a β-elimination step which results in cleavage of a bond between the abasic site deoxyribose ring and its 3′ phosphate, generating a reactive α,β-unsaturated aldehyde which can be labeled or can undergo further cleavage and cyclization reactions. See, e.g. Sugiyama, Chem. Res. Toxicol. (1994) 7: 673-83; Horn, Nucl. Acids. Res., (1988) 16:11559-71. It is understood that more than one method of cleavage can be used, including two or more different methods which result in multiple, different types of cleavage products (e.g., fragments comprising an abasic site at the 3′ end, and fragments comprising an abasic site at the 5′ end).

Generally, cleavage of the backbone at an abasic site is general, specific or selective cleavage (in the sense that the agent (such as an enzyme) capable of cleaving the backbone at an abasic site specifically or selectively cleaves the base portion of a particular non-canonical nucleotide), whereby greater than about 98%, about 95%, about 90%, about 85%, or about 80% of the cleavage is at an abasic site. However, extent of cleavage can be less. Thus, reference to specific cleavage is exemplary. General, specific or selective cleavage is desirable for control of the fragment size in the methods of generating labeled polynucleotide fragments of the invention. In some embodiments, reaction conditions can be selected such that the cleavage reaction is performed in the presence of a large excess of reagents and allowed to run to completion with minimal concern about excessive cleavage of the polynucleotide (i.e., while retaining a desired fragment size, which is determined by spacing of the incorporated non-canonical nucleotide, during the synthesis step, above). In other embodiments,-extent of cleavage can be less, such that polynucleotide fragments are generated comprising an abasic site at an end and an abasic site(s) within or internal to the polynucleotide fragment (i.e., not at an end). As disclosed herein, polynucleotide fragments comprising internal abasic sites are useful e.g., in embodiments involving immobilization of a labeled polynucleotide (wherein one abasic site is used for immobilization and another abasic site(s) are labeled at an abasic site).

As noted herein, the frequency of incorporation of non-canonical nucleotides into the polynucleotide relates to the size of fragment produced using the methods of the invention because the spacing between non-canonical nucleotides in the polynucleotide comprising a non-canonical nucleotide, as well as the reaction conditions selected, determines the approximate size of the resulting fragments (following cleavage of a base portion of a non-canonical nucleotide, whereby an abasic site is generated, and cleavage of the backbone at the abasic site as described herein). Generally, suitable fragment sizes are about 5, 10, 15; 20, 25, 30, 40, 50, 65, 75, 85, 100, 123, 150, 175, 200, 225, 250, 300, 350, 400, 450, 500, 550, 600, 650 or more nucleotides in length. In some embodiments, the fragment is about 200 nucleotides, about 100 nucleotides, or about 50 nucleotides in length. In another embodiment, the size of a population of fragments is about 50 to 200 nucleotides. It is understood that the fragment size is approximate, particularly when populations of fragments are generated, because the incorporation of a non-canonical nucleotide (which relates to the fragment size following cleavage) will vary from template to template, and also between copies of the same template. Thus, fragments generated from same starting material (such as a single polynucleotide template) may have different (and/or overlapping) sequence, while still having the same approximate size or size range.

Following cleavage of the polynucleotide backbone at the abasic site, every fragment will comprise one abasic site (if cleavage is completely efficient), except for either the 5′- or 3′-most fragment, which will lack an abasic site depending on the cleavage agent. If the cleavage is 5′ to the abasic site, the 5′ most fragment will not comprise an abasic site. If cleavage is 3′ to the abasic site, the 3′ most fragment will not comprise an abasic site. If it is desired to incorporate an abasic site into a 5′-most fragment, (if the synthesis step requires a primer(s)), a primer comprising a non-canonical nucleotide can be used, as discussed herein, and the resulting abasic site in the primer will be cleaved. Generally, if cleavage of the phosphodiester backbone is 5′ to the abasic residue, the abasic site should be incorporated at the 5′ end of the primer (or the DNA portion of the primer, if an RNA-DNA composite primer is used, see Kurn, U.S. Pat. No. 6,251,639 B1).

Labeling the Abasic Site and Detection

The abasic site is labeled, whereby a polynucleotide (or polynucleotide fragment) comprising a label is generated. In some embodiments, a polynucleotide fragment comprising an abasic site is contacted with an agent capable of labeling at the abasic site; whereby labeled fragments of the polynucleotide are generated. As used herein, a “label” (interchangeably called a “detectable moiety”) is associated with a polynucleotide, such that the polynucleotide comprising an abasic site is associated with a label.

Thus, in some embodiments, the label associates with a chemical structure remaining following treatment of a non-canonical nucleotide (present in a polynucleotide chain) with an agent (e.g., an enzyme, or acidic conditions, or a chemical reagent) capable of effecting cleavage of a base portion of a non-canonical nucleotide. In embodiments involving fragmentation, the label associates with any chemical structure remaining following treatment of a non-canonical nucleotide (present in a polynucleotide chain) with an agent (e.g., an enzyme, or acidic conditions, or a chemical reagent) capable of effecting cleavage of a base portion of a non-canonical nucleotide, and following treatment with an agent capable of cleaving the backbone at the abasic site. In one embodiment, the label covalently bonds with a reactive aldehyde form of a hemiacetal ring in an abasic site. In some embodiments, labeling “at” an abasic site encompasses labels that bind to an abasic site, but do not bind to the intact (uncleaved) non-canonical nucleotide (whether incorporated or present as a single non-canonical nucleotide). In some embodiment, labeling “at” an abasic site specifically excludes labels that associate (e.g., covalently bind) with a phosphate group of a nucleotide (or polynucleotide) or a phosphate group of an abasic site. As made clear from the disclosure herein, “label” refers to any component of a labeling system.

The embodiment shown in FIG. 1 illustrates cleavage of the phosphodiester backbone at an abasic site of the polynucleotide comprising the abasic site, whereby a cleaved polynucleotide fragment is produced, then covalently or non-covalently associating a label with the cleaved fragment, such that labeled polynucleotide fragments are generated. It is understood that cleavage of the phosphodiester backbone at the abasic site, and labeling at an abasic site can be performed in any order, or simultaneously (for example, as disclosed in Example 4, herein).

The label can be detectable, or the label can be indirectly detected, for example as when the label (attached at an abasic residue) is covalently or non-covalently associated with another moiety which is itself detected. For example, biotin can be attached to the label capable of associating with the abasic site. In another example, an antibody (that can be detectably labeled) binds the label that is attached at the abasic site. In some embodiments, the label comprises an organic molecule, a hapten, or a particle (such as a polystyrene bead). In some embodiments, the label is detected using antibody binding, biotin binding, or via fluorescence or enzyme activity. In some embodiments, the detectable signal is amplified.

Generally, labeling at an abasic site is general, specific, or selective labeling (in the sense that the agent capable of labeling at an abasic site specifically or selectively labels the abasic site), whereby greater than about 98%, about 95%, about 93%, about 90%, about 85%, or about 80% of the labels bind abasic sites. However, extent of labeling can be less. Thus, reference to specific labeling is exemplary. Generally, reaction conditions are selected such that the reaction in which the abasic site(s) are labeled can run to completion.

In some embodiments, labeled polynucleotide fragments are produced which each comprise a single label (to the extent that cleavage of the phosphodiester backbone is generally complete, in the sense that many or essentially all of the polynucleotide fragments comprise a single abasic site). This aspect is useful in quantitating level of hybridization, because signal is proportional to number of bound fragments, and does not relate to the length of the hybridizing fragment or the number of labels per fragment. Thus, hybridization intensity can generally be directly compared, regardless of fragment length. This offers an advantage over prior art methods in which nucleic acid fragments are labeled with multiple detectable moieties, e.g., incorporation of labeled nucleotides, and other methods of directly and indirectly detecting incorporated nucleotides. These methods generally result in multiple labels per hybridizing fragment, and thus are generally less suitable for quantitative applications. Multiple labels per nucleic acid can result in quenching, and potential interference with hybridization kinetics (due to the presence of multiple labeled moieties per nucleic acid).

In another embodiment, labeled fragments are produced which comprise a labeled abasic site at an end (such as the 3′ end and/or the 5′ end) and a labeled internal abasic site.

Methods and reaction conditions for labeling abasic sites are known in the art. For example, a common functional group exposed in an abasic site (and therefore suitable for use in labeling) is the highly reactive aldehyde form of the hemiacetal ring which can be covalently or noncovalently attached to a label using reaction conditions that are known in the art. Many labels comprise substituted hydrazines or hydroxylamines which readily form imine bonds with aldehydes, for example, 5-(((2-(carbohydrazino)-methyl)thio)acetyl)aminofluorescein, aminooxyacetyl hydrazide (FARP). See Makrogiorgos, WO 00/39345. The stable oxime formed by this compound can be detected directly by fluorescence or the signal can be amplified using an antibody-enzyme conjugate. See, e.g., Srivastava, J. Biol. Chem. (1998) 273(33): 21203-209; Makrigiorgos, Int J. Radiat. Biol. (1998) 74(1):99-109; Makriogiorgos, U.S. Pat. No. 6,174,680 B1; Makrogiorgos, WO 00/39345. Suitable sidechains (present on the substrate) to react with the aldehyde (of the abasic site) include at least the following: substituted hydrazines, hydrazides, or hydroxylamines (which readily form imine bonds with aldehydes), and the related semicarbazide and thiosemicarbazide groups, and other amines which can form stable carbon-nitrogen double bonds, that can catalyze simultaneous cleavage and binding (see Horn, Nucl. Acids. Res., (1988) 16:11559-71), or can be coupled to form stable conjugates, e.g. by reductive amination. Other methods for attaching a reactive group present in an abasic site to a reactive group present on a label are known in the art. In another example, the abasic site may be chemically modified, then the modified abasic site covalently or non-covalently attached to a suitable reactive group on a substrate. For example, the aldehyde (in the abasic site) can be oxidized or reduced (using methods known in the art), then covalently immobilized to a substrate using, e.g., reductive amination or various oxidative processes.

Other suitable reagents are known in the art, e.g., fluorescein aldehyde reagents. See, e.g., Boturyn (1999) Chem. Res. Toxicol. 12:476-482. See, also, Adamczyk (1998) Bioorg. Med. Chem. Lett. 8(24):3599-3602; Adamczyk (1999) Org. Lett. 1(5):779-781; Kow (2000) Methods 22(2):164-169; Molecular Probes Handbook, Section 3.2 (www.probes.com). For example, detectable moieties comprising aminooxy groups can be used. See, Boturyn, supra. The aminoooxy group readily reacts with the highly reactive aldehyde form of the hemiacetal ring of an abasic site. In one embodiment, the label comprising an aminooxy reactive group is N-(aminooxyacetyl)-N′-(D-biotinoyl) hydrazine, trifluoroacetic acid salt (ARP) (available from Molecular Probes, Eugene Oreg., catalog No. A-10550). See, e.g., Kubo et al., Biochem 31:3703-3708 (1992); Ide et al., Biochem. 32:8276-8283 (1993).

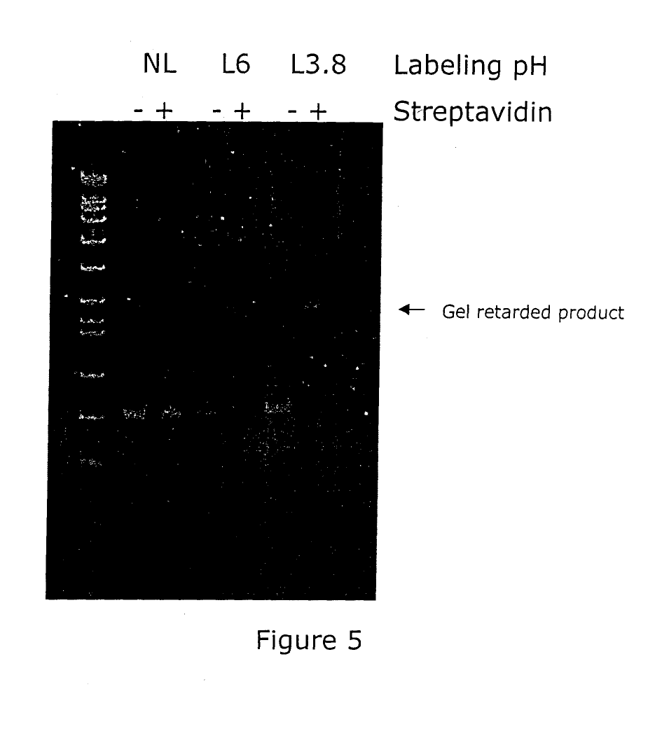

In yet another example, labels comprising a hydrazide linker can be converted to an aminooxy derivative, then used to label abasic sites as described herein. In one embodiment, the label comprises an aminooxy derivatized Alexa Fluor 555 reagent. As shown in FIG. 5, use of the aminooxy-derivatized Alexa Fluor 555 resulted in greater labeling efficiency, as well as increased fluorescence as compared to labeling with unmodified Alexa Fluor 555 hydrazide (Order No. A-20501, Molecule Probes, Eugene Oreg.).

In another example, the abasic site may be chemically modified (before, during or after cleavage of the phosphodiester backbone as described herein), then the modified abasic site detected directly or indirectly. For example, fluorescent cadaverine can be incorporated into an abasic site as described in Horn (Nucl. Acids. Res., (1988) 16:11559-71). In another example, the abasic site may be chemically modified by reaction with NHBA (0-4-nitrobenzyl hydroxylamine), then the NBHA-modified abasic site is detected with an antibody that specifically binds to the NBHA-modified abasic sites See Kow et al, WO 92/07951 (1992).

In another example, the abasic site may be labeled with an antibody (such as a monoclonal or polyclonal antibody or antigen binding fragment). Methods for detecting specific antibody binding are well known in the art.

In another example, the aldehyde and/or hemiacetal ring may itself be detected, as when for example, detectable signal is generated using chemical or electrochemical reactions specific to those chemical structures, including for example, oxidation reactions, enzymes with dehydrogenase or oxidase activity, and the like. In another example, many aldehydes are substrates for enzymes, such that a detectable product is generated in the presence of the aldehyde. For example, dehydrogenases typically couple oxidation of an aldehyde with reduction of NAD+ which can be detected spectrophotometrically. In another example, glucose oxidases generate hydrogen peroxide in the presence of sugar aldehydes. Hydrogen peroxide is readily detectable by coupling to horseradish peroxidase with suitable substrates. Thus, the invention provides methods for detecting an abasic site.

Methods of signal detection are known in the art. Signal detection may be visual or utilize a suitable instrument appropriate to the particular label used, such as a spectrometer, fluorimeter, or microscope. For example, where the label is a radioisotope, detection can be achieved using, for example, a scintillation counter, or photographic film as in autoradiography. Where a fluorescent label is used, detection may be by exciting the fluorochrome with the appropriate wavelength of light and detecting the resulting fluorescence, such as by microscopy, visual inspection or photographic film, fluorometer, CCD cameras, scanner and the like. Where enzymatic labels are used, detection may be by providing appropriate substrates for the enzyme and detecting the resulting reaction product. For example, many substrates of horseradish peroxidase, such as o-phenylenediamine, give colored products. Simple colorimetric labels can usually be detected by visual observation of the color associated with the label; for example, conjugated colloidal gold is often pink to reddish, and beads appear the color of the bead. Instruments suitable for high sensitivity detection are known in the art.

It is understood that the polynucleotide or polynucleotide fragments can be additionally labeled using other methods known in the art, such as incorporation of labeled nucleotide analogs during synthesis of the polynucleotide comprising a non-canonical nucleotide. In addition, following cleavage of the phosphodiester backbone of the polynucleotide comprising an abasic site, either the 5′ most or the 3′ most fragment will lack an abasic site, depending on the cleavage agent (in embodiments in which the fragmentation reaction goes to completion). However, as discussed herein, if the synthesis step requires primer(s), a labeled primer(s) can be used such that the resulting fragment comprising a primer is labeled. Suitable labels and methods of labeling primers are known. In addition, a primer comprising a non-canonical nucleotide can be used. Following generation of an abasic site, cleavage of the phosphodiester backbone at the abasic site, and labeling at the abasic site, the fragment comprising at least a portion of the primer will be labeled. Generally, if cleavage of the phosphodiester backbone is 5′ to the abasic residue, the abasic site should be incorporated at the 5′ end of the primer (or the DNA portion of the primer, if a composite primer is used, see Kurn, U.S. Pat. No. 6,251,639 B1); U.S. Patent Publication No. 2003/0087251 A1.

Labeled polynucleotide fragments can be immobilized to a substrate, as described herein.

Methods for Labeling Nucleic Acids

The invention provides methods for generating labeled nucleic acid(s). The methods generally comprise generation of a polynucleotide comprising at least one non-canonical nucleotide, cleavage of a base portion of the non-canonical nucleotide present in the polynucleotide with an agent capable of cleaving a base portion of the non-canonical nucleotide; and labeling the abasic site, whereby labeled polynucleotide(s) is generated. Generally, the polynucleotide comprising a non-canonical nucleotide is labeled at the site of incorporation of non-canonical nucleotides in the polynucleotide (following generation of an abasic site by cleavage of a base portion of a non-canonical nucleotide). The methods of the invention generate labeled polynucleotide(s), which are useful for, for example, hybridization to a microarray and other uses described herein.

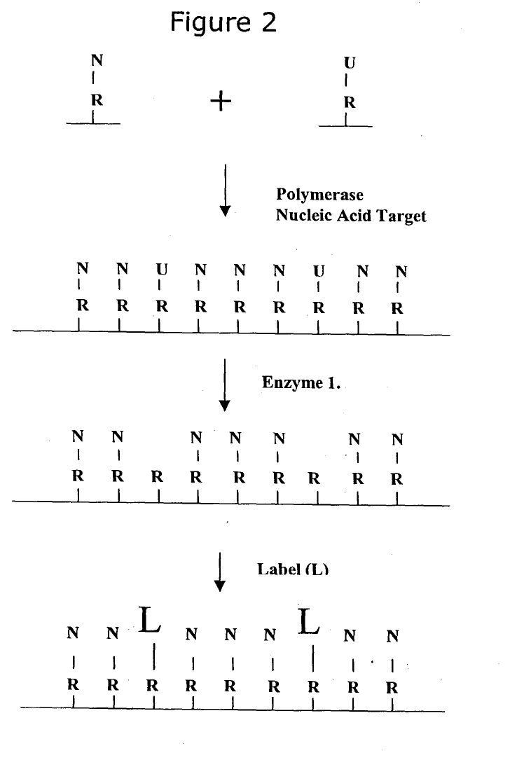

The methods involve the following steps: (a) synthesizing a polynucleotide from a template in the presence of at least one non-canonical nucleotide (interchangeably termed “non-canonical deoxyribonucleoside triphosphate”), whereby a polynucleotide comprising a non-canonical nucleotide is generated; (b) contacting the polynucleotide comprising a non-canonical nucleotide with an agent capable of cleaving a base portion of the non-canonical nucleotide, whereby an abasic site is created; and (c) labeling the abasic site in the polynucleotide comprising the abasic site, whereby labeled polynucleotide(s) is generated. A schematic description of one embodiment of the labeling methods of the invention is given in FIG. 2.

For simplicity, individual steps of the labeling methods are discussed below. It is understood, however, that the steps may be performed simultaneously and in varied order, as discussed herein.

Synthesis of a Polynucleotide Comprising a Non-Canonical Nucleotide

The methods involve synthesizing a polynucleotide from a template in the presence of at least one non-canonical nucleotide, whereby a polynucleotide comprising a non-canonical nucleotide is generated. The exemplary embodiment illustrated in FIG. 2 illustrates synthesis of a single stranded polynucleotide from a template in the presence of non-canonical nucleotides, such that a single stranded polynucleotide comprising the non-canonical nucleotide is generated. The frequency of incorporation of non-canonical nucleotides into the polynucleotide relates to the frequency of labeled abasic site generated using the methods of the invention because the spacing between non-canonical nucleotides in the polynucleotide comprising a non-canonical nucleotide determines the approximate spacing of the labeled sites in the labeled nucleic acid.

Generally, the polynucleotide is DNA, though, as noted herein, the polynucleotide can comprise altered and/or modified nucleotides, internucleotide linkages, ribonucleotides, etc. As generally used herein, it is understood that “DNA” applies to polynucleotide embodiments.

Methods for synthesizing polynucleotides, e.g., single and double stranded DNA, from a template are well known in the art, and are described herein. For convenience, “DNA” is used herein to describe (and exemplify) a polynucleotide.

Generally, single or double stranded polynucleotide is generated from a template in the presence of all four canonical nucleotides and at least one non-canonical nucleotide under reaction conditions suitable for synthesis of DNA, including suitable enzymes and primers, if necessary. Reaction conditions and reagents, including primers, for synthesizing the polynucleotide comprising a non-canonical nucleotide are known in the art, and discussed herein. As described herein, non-canonical nucleotides are generally capable of polymerization, and capable of being rendered abasic following treatment with a suitable agent capable of generally, specifically or selectively cleaving a base portion of a non-canonical nucleotide. Suitable non-canonical nucleotides are well-known in the art and are described herein. In some embodiments, the polynucleotide comprising a non-canonical nucleotide is synthesized using single primer isothermal amplification, see Kurn, U.S. Pat. No. 6,251,639 B1; Kurn, WO02/00938; and/or Ribo-SPIATM, see Kurn, U.S. Patent Publication No. 2003/0087251 A1.

Conditions for limited and/or controlled incorporation of a non-canonical nucleotide are known in the art and are described herein. The frequency (or proportion) of non-canonical bases in the resulting polynucleotide comprising a non-canonical nucleotide, and thus the frequency of labeling in the labeled polynucleotide, is controlled by variables known in the art, including: frequency of nucleotide(s) corresponding to the non-canonical nucleotide(s) in the template (or other measures of nucleotide content of a sequence, such as average G-C content), ratio of canonical to non-canonical nucleotide present in the reaction mixture; ability of the polymerase to incorporate the non-canonical nucleotide, relative efficiency of incorporation of non-canonical nucleotide verses canonical nucleotide, and the like.

Generally, the polynucleotide comprising a non-canonical nucleotide is labeled at the site of incorporation of the non-canonical nucleotide(s) (i.e., at an abasic site, as described herein) present in the synthesized polynucleotide. Thus, the frequency of non-canonical nucleotides in the synthesized polynucleotide generally determines the frequency of labels in the labeled polynucleotide. Generally, a non-canonical base can be incorporated at about every 5, 10, 15, 20, 25, 30, 40, 50, 65, 75, 85, 100, 123, 150, 175, 200, 225, 250, 300, 350, 400, 450, 500, 550, 600, 650 or more nucleotides apart in the resulting polynucleotide comprising a non-canonical nucleotide. In one embodiment, the non-canonical nucleotide is incorporated about every 500 nucleotides. In one embodiment, the non-canonical nucleotide is incorporated about every 100 nucleotides. In another embodiment, the non-canonical nucleotide is incorporated about every 50 nucleotides. In another embodiments, the non-canonical nucleotide is incorporated about every 50 to 200 nucleotides. It is understood that these length generally represent average lengths in a population of polynucleotides generated using the methods of the invention.

Methods of synthesis are generally template-dependent (as described herein). However, it is understood that non-canonical nucleotides can be incorporated into a polynucleotide as a result of template-independent methods (e.g. ligation, tailing), as described herein.

The template may be any template from which labeled polynucleotides are desired to be produced. The template includes double-stranded, partially double stranded and single-stranded nucleic acids from any source in purified or unpurified form, as described herein.

For simplicity, the polynucleotide comprising a non-canonical nucleotide is described as a single nucleic acid. It is understood, however, that the polynucleotide comprising a non-canonical nucleotide can be a single nucleic acid, for example, as produced by reverse transcription, first and second strand cDNA production, or a single cycle of DNA replication. The polynucleotide can also be a population of amplified products (from a few to very many), for example single stranded DNA products produced using single primer isothermal amplification and/or Ribo-SPIATM, see Kurn, U.S. Pat. No. 6,251,639 B1; Kurn, U.S. Patent Publication No. 2003/0087251 A1, or double stranded DNA product produced by, for example, PCR. It is further understood that a polynucleotide comprising a non-canonical nucleotide can be a multiplicity (from small to very large) of different polynucleotide molecules. Such populations can be related in sequence (e.g., member of a gene family or superfamily) or extremely diverse in sequence (e.g., generated from all mRNA, generated from all genomic DNA, etc.). Polynucleotides can also correspond to single sequence (which can be part or all of a known gene, for example a coding region, genomic portion, etc.). Methods, reagents, and reaction conditions for generating specific polynucleotide sequences and multiplicities of polynucleotide sequences are known in the art

Cleaving a Base Portion of the Non-Canonical Nucleotide to Create an Abasic Site

The polynucleotide comprising a non-canonical nucleotide (synthesized from a template, as described herein) is treated with an agent (such as an enzyme) capable of generally, specifically or selectively cleaving a base portion of the non-canonical nucleotide to create an abasic site. The embodiment shown in FIG. 2 illustrates cleavage of a base portion of the non-canonical nucleotides, whereby an abasic site is created. In some embodiments, the agent (such as an enzyme) catalyzes hydrolysis of the bond between the base portion of the non-canonical nucleotide and a sugar in the non-canonical nucleotide to generate an abasic site comprising a hemiacetal ring and lacking the base (interchangeably called “AP” site), though other cleavage products are contemplated for use in the methods of the invention. Suitable agents and reaction conditions for cleavage of base portions of non-canonical nucleotides are known in the art and are described herein. In one embodiment, uracil-N-glycosylase is used to cleave a base portion of the non-canonical nucleotide.

Generally, cleavage of base portions of non-canonical nucleotides is general, specific or selective cleavage, whereby greater than about 98%, about 95%, about 90%, about 85%, or about 80% of the base portions cleaved are base portions of non-canonical nucleotides. However, extent of cleavage can be less. Thus, reference to specific cleavage is exemplary. General, specific or selective cleavage is desirable for control of the number of potential labeling sites (and thus the intensity of labeling) in the methods of generating labeled polynucleotides of the invention. Generally, reaction conditions are selected such that the reaction in which the abasic site(s) are created can run to completion.

For convenience, the synthesis of a polynucleotide comprising a non-canonical nucleotide, and the cleavage of that polynucleotide by an enzyme capable of cleaving a base portion of the non-canonical nucleotide are described as separate steps. It is understood that these steps may be performed simultaneously, except (generally) in the case when a polynucleotide comprising a non-canonical nucleotide must be capable of serving as a template for further amplification (as in exponential methods of amplification, e.g. PCR).

Labeling the Abasic Site and Detection

The abasic site is labeled, whereby a polynucleotide (or polynucleotide fragment) comprising a detectable moiety is generated. The embodiment shown in FIG. 2 illustrates labeling at the abasic sites of a single stranded polynucleotide comprising abasic sites, such that labeled polynucleotides are produced. As used herein, “detectable moiety” (interchangeably called a label) refers to a covalent or non-covalent association of agent (interchangeably called “labeling”) with an abasic site in a polynucleotide such that polynucleotides comprising an abasic site are associated with a detectable signal. Accordingly, in some embodiments, the detectable moiety (label) is covalently or non-covalently associated with an abasic site. In some embodiments, the detectable moiety (label) is directly or indirectly detectable. In some embodiments, the detectable signal is amplified. In some embodiments, the detectable moiety comprises an organic molecule. In other embodiments, the detectable moiety comprises an antibody. In other embodiments, the detectable signal is fluorescent. In other embodiments, the detectable signal is enzymatically generated. Other labeling embodiments are described herein.

Generally, labeling at an abasic site is general, specific or selective labeling (in the sense that the agent capable of labeling at an abasic site specifically or selectively labels the abasic site), whereby greater than about 98%, about 95%, about 93%, about 90%, about 85%, or about 80% of the labels bind abasic sites. However, extent of labeling can be less. Thus, reference to specific labeling is exemplary. Generally, reaction conditions are selected such that the reaction in which the abasic site(s) are labeled can run to completion.

Methods and reaction conditions for generally, specifically or selectively labeling abasic sites are known in the art and are described herein. Generally, methods for labeling abasic site which also result in cleavage of a phosphodiester backbone should be avoided, unless simultaneous cleavage and labeling is desired (see, e.g., Horn, Nucl. Acids. Res. (1988) 16:11559-71).

In some embodiments, labeled polynucleotide fragments are produced which each comprise a single label (to the extent that cleavage of the phosphodiester backbone is generally complete, in the sense that many or essentially all of the polynucleotide fragments comprise a single abasic site). In another embodiment, labeled fragments are produced which comprise a labeled abasic site at an end (such as the 3′ end and/or the 5′ end) and a labeled internal abasic site.

Methods of detecting detectable signals are known in the art and are described herein. Signal detection may be visual or utilize a suitable instrument) appropriate to the particular label used, such as a spectrometer, fluorometer, or microscope. For example, where the label is a radioisotope, detection can be achieved using, for example, a scintillation counter, or photographic film as in autoradiography. Where a fluorescent label is used, detection may be by exciting the fluorochrome with the appropriate wavelength of light and detecting the resulting fluorescence, such as by microscopy, visual inspection or photographic film. Where enzymatic labels are used, detection may be by providing appropriate substrates for the enzyme and detecting the resulting reaction product. Simple colorimetric labels can usually be detected by visual observation of the color associated with the label; for example, conjugated colloidal gold is often pink to reddish, and beads appear the color of the bead.

It is understood that the polynucleotide or polynucleotide can be additionally labeled using other methods known in the art, such as incorporation of labeled nucleotide analogs during synthesis of the polynucleotide comprising a non-canonical nucleotide. If the synthesis step requires primer(s), a labeled primer(s) can be used. Suitable labels and methods of labeling primers are known. In addition, a primer comprising a non-canonical nucleotide can be used. Following generation of an abasic site, cleavage of the phosphodiester backbone at the abasic site, and labeling at the abasic site, the primer will be labeled.

Labeled polynucleotide can be immobilized to a substrate as described herein.

Methods for Preparing Polynucleotides (or Fragments Thereof) Immobilized on a Substrate

The invention provides methods for generating polynucleotides or polynucleotide fragments immobilized on a substrate (interchangeably termed a “surface”, herein). The methods generally comprise immobilization of a polynucleotide comprising an abasic site, or fragments thereof (in embodiments involving fragmentation), to a substrate at the abasic site. In some embodiments, the methods provide cleavage of a base portion of a non-canonical nucleotide present in a polynucleotide with an agent capable of cleaving a base portion of the non-canonical nucleotide, whereby an abasic site is created; optionally cleaving the phosphodiester backbone of the polynucleotide at the abasic site, whereby fragments of the synthesized nucleic acid are generated; and immobilizing the polynucleotide, or fragments thereof, on a substrate, wherein the polynucleotide or fragment thereof is immobilized at the abasic site. Optionally, the polynucleotide comprising an abasic site can be labeled at an abasic site according to the labeling methods described herein. The labeling may be anywhere on the immobilized fragment (for example, as when an internal abasic site is labeled, or an abasic site at a terminus of the polynucleotide is labeled). Generally, the polynucleotide comprising an abasic site is immobilized at the abasic site in the polynucleotide. Thus, as discussed above, the frequency of non-canonical nucleotides in the synthesized polynucleotide generally determines the number of abasic sites available for immobilization to a substrate (and the size range of the fragments produced from the polynucleotide, in embodiments involving cleavage of the phosphodiester backbone). The methods of the invention generate polynucleotides, and fragments thereof, immobilized on a substrate, for example, a microarray. In some embodiments, one or more abasic site(s) are labeled (as described herein) and one or more abasic site(s) are immobilized to a substrate.

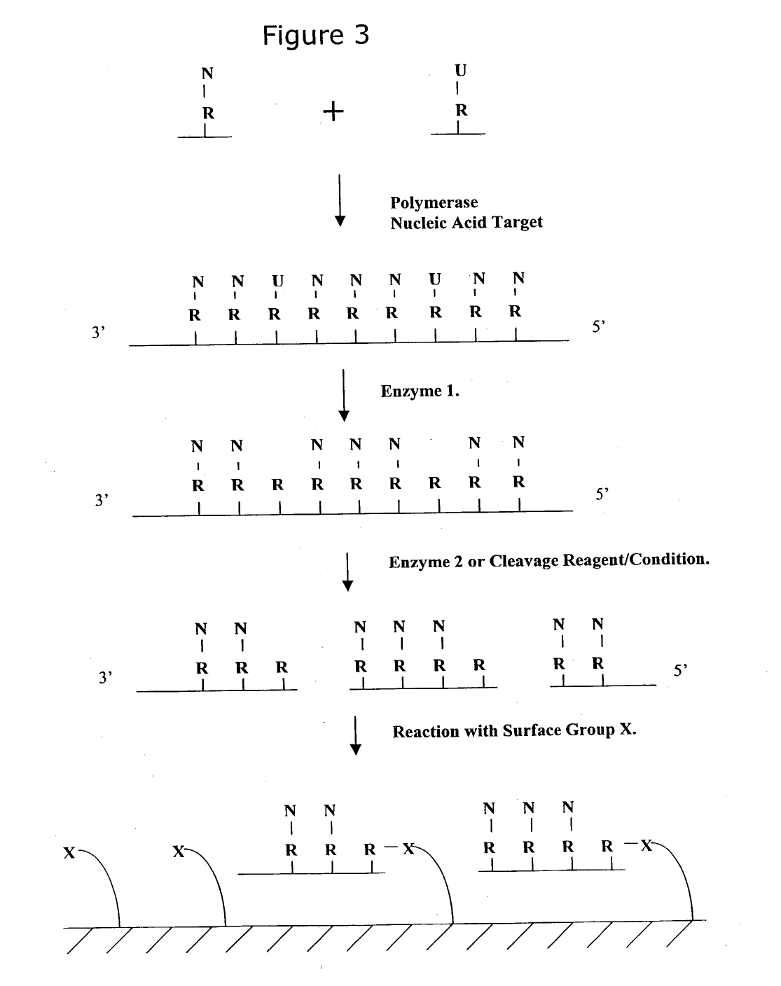

The methods involve the following steps: (a) contacting a polynucleotide comprising a non-canonical nucleotide with an agent capable of cleaving a base portion of the non-canonical nucleotide, whereby an abasic site is created; (b) optionally cleaving a phosphodiester backbone at the abasic site; whereby fragments of the synthesized nucleic acid are generated; (c) optionally labeling a polynucleotide at the abasic site; and (d) immobilizing the polynucleotide (or polynucleotide fragments) on a substrate, wherein the polynucleotide is immobilized to the substrate through the abasic site. In some embodiments, the polynucleotide comprising a non-canonical nucleotide is synthesized from a template in the presence of at least one non-canonical nucleotide. In some embodiments, the polynucleotide comprising a non-canonical nucleotide is synthesized using single primer isothermal TM amplification or Ribo-SPIA™. See Kurn, U.S. Pat. No. 6,251,639 B1; Kurn, WO02/00938; Kurn, U.S. Patent Publication No. 2003/0087251 A1. A schematic description of one embodiment of the immobilization methods of the invention is given in FIG. 3.

For simplicity, individual steps of the methods are discussed below. It is understood, however, that the steps may be performed simultaneously and in varied order, as discussed herein. It is also understood that the invention encompasses methods in which the initial, or first, step is any of the steps described herein. For example, the method encompasses embodiments wherein a polynucleotide comprising an abasic site, or a polynucleotide fragment comprising an abasic site, are immobilized to a substrate as described herein.

Preparation of a Polynucleotide Comprising a Non-Canonical Nucleotide

In some embodiments, the polynucleotide comprising a non-canonical nucleotide is synthesized from a template in the presence of at least one non-canonical nucleotide, as discussed herein. The embodiment illustrated in FIG. 3 illustrates synthesis of a single stranded polynucleotide from a template in the presence of non-canonical nucleotides, such that a single stranded polynucleotide comprising the non-canonical nucleotide is generated, though other embodiments are contemplated by the methods of the invention. Other methods for generating a polynucleotide comprising a non-canonical nucleotide are well known in the art, including tailing, ligation, oligonucleotide synthesis, and the like. See, e.g., Sambrook (1989) “Molecular Cloning: A Laboratory Manual”, second edition; Ausebel (1987, and updates) “Current Protocols in Molecular Biology”.

Generally, the polynucleotide is DNA, though, as noted herein, the polynucleotide can comprise altered and/or modified nucleotides, internucleotide linkages, ribonucleotides, etc. As generally used herein, it is understood that “DNA” applies to polynucleotide embodiments.

Methods for synthesizing polynucleotides, e.g., single and double stranded DNA, are well known in the art, and include template-dependent and template-independent methods. Examples of template-dependent methods include, for example, single primer isothermal amplification, Ribo-SPIA™, PCR, reverse transcription, primer extension, limited primer extension, replication (including rolling circle replication), strand displacement amplification (SDA), nick translation and, e.g., any method that results in synthesis of the complement of a template sequence such that at least one non-canonical nucleotide can be incorporated into a polynucleotide. See, e.g., Kurn, U.S. Pat. No. 6,251,639 B1; Kurn, WO02/00938; Kurn, U.S. Patent Publication No. 2003/0087251 A1; Mullis, U.S. Pat. No. 4,582,877; Wallace, U.S. Pat. Nos. 6,027,923; 5,508,178; 5,888,819; 6,004,744; 5,882,867; 5,710,028; 6,027,889; 6,004,745; 5,763,178; 5,011,769; see also Sambrook (1989) “Molecular Cloning: A Laboratory Manual”, second edition; Ausebel (1987, and updates) “Current Protocols in Molecular Biology”; Mullis, (1994) “PCR: The Polymerase Chain Reaction”. In one embodiment, the polynucleotide comprising a non-canonical nucleotide is synthesized using single primer isothermal amplification and/or Ribo-SPIA™. See Kurn, U.S. Pat. No. 6,251,639 B1; Kurn, WO02/00938; Kurn, U.S. Patent Publication No. 2003/0087251 A1. Methods of template independent methods include oligonucleotide synthesis, ligation, and tailing, as described herein.

Suitable methods include methods that result in one single- or double-stranded (or partially double stranded) polynucleotide comprising a non-canonical nucleotide (for example, reverse transcription, production of double stranded cDNA, a single round of DNA replication), as well as methods that result in multiple single stranded or double stranded copies or copies of the complement of a template (for example, single primer isothermal amplification, Ribo-SPIA™ or PCR). See Kurn, U.S. Pat. No. 6,251,639 B1; Kurn, WO02/00938; Kurn, U.S. Patent Publication No. 2003/0087251 A1. One or more methods known in the art can be used to generate a polynucleotide comprising a non-canonical nucleotide. It is understood that the polynucleotide comprising a non-canonical nucleotide can be single-stranded or double-stranded, e.g. single and double stranded DNA, or partially double stranded, and that each strand of a double-stranded polynucleotide can comprises a non-canonical nucleotide. For convenience, “DNA” is used herein to describe (and exemplify) a polynucleotide.

Reaction conditions and reagents, including primers, for producing the polynucleotide comprising a non-canonical nucleotide are known in the art, and described herein (see, e.g., methods for synthesizing a polynucleotide comprising an abasic site, as described herein).

Generally, the polynucleotide comprising an abasic site is immobilized to a substrate at the abasic sites, as described herein. Thus, the frequency of non-canonical nucleotides in the polynucleotide relates to the frequency of abasic site generated (in the polynucleotide comprising the non-canonical nucleotide following cleavage of a base portion of the non-canonical nucleotide), and thus the number of abasic sites in the polynucleotide comprising abasic site(s) useful (or available) for immobilization of the polynucleotide according to the method of the invention. Generally, a non-canonical base can be incorporated at about every 5, 10, 15, 20, 25, 30, 40, 50, 65, 75, 85, 100, 123, 150, 175, 200, 225, 250, 300, 350, 400, 450, 500, 550, 600, 650 or more nucleotides apart in the resulting polynucleotide comprising a non-canonical nucleotide. In one embodiment, the non-canonical nucleotide is incorporated about every 500 nucleotides. In one embodiment, the non-canonical nucleotide is incorporated about every 100 nucleotides. In another embodiment, the non-canonical nucleotide is incorporated about every 50 nucleotides. In still other embodiments, the non-canonical nucleotide is incorporated about every 50 to 200 nucleotides. It is understood that these length generally represent average lengths in a population of polynucleotides (or fragments thereof in embodiments involving fragmentation) generated using the methods of the invention.

In some embodiments, the polynucleotide comprising a non-canonical nucleotide is cleaved at the non-canonical nucleotide(s) (i.e., at an abasic site following cleavage of a base portion of the non-canonical nucleotide) present in the synthesized polynucleotide. Thus, the frequency of non-canonical nucleotides in the polynucleotide generally determines the size range of the fragments produced from the polynucleotide. Generally, a non-canonical nucleotide can be present at about every 5, 10, 15, 20, 25, 30, 40, 50, 65, 75, 85, 100, 123, 150, 175, 200, 225, 250, 300, 350, 400, 450, 500, 550, 600, 650 or more nucleotides apart in the resulting polynucleotide comprising a non-canonical nucleotide. In one embodiment, the non-canonical nucleotide is incorporated about every 500 nucleotides. In one embodiment, the non-canonical nucleotide is incorporated about every 100 nucleotides. In another embodiment, the non-canonical nucleotide is incorporated about every 50 nucleotides. In still another embodiment, the non-canonical nucleotide is incorporated about every 50 to about every 200 nucleotides. It is understood that these length generally represent average lengths in a population of polynucleotides (or fragments thereof in embodiments involving fragmentation) generated using the methods of the invention. Conditions for limited and/or controlled incorporation of a non-canonical nucleotide are known in the art and are described herein. The frequency (or proportion) of non-canonical bases in the resulting polynucleotide comprising a non-canonical nucleotide, and thus the average size of fragments generated using the methods of the invention (i.e., following cleavage of a base portion of a non-canonical nucleotide, and cleavage of a phosphodiester bond at a non-canonical nucleotide), is controlled by variables known in the art, including: frequency of nucleotide(s) corresponding to the non-canonical nucleotide(s) in the template (or other measures of nucleotide content of a sequence, such as average G-C content), ratio of canonical to non-canonical nucleotide present in the reaction mixture; ability of the polymerase to incorporate the non-canonical nucleotide, relative efficiency of incorporation of non-canonical nucleotide verses canonical nucleotide, and the like. The reaction conditions can be empirically determined, for example, by assessing average fragment size generated using the methods of the invention taught herein.

The template may be any template from which immobilized polynucleotides (polynucleotide fragments) are desired to be produced, as described herein.

For simplicity, the polynucleotide comprising a non-canonical nucleotide is described as a single nucleic acid. It is understood, however, that the polynucleotide comprising a non-canonical nucleotide can be a single nucleic acid, for example, as produced by reverse transcription, first and second strand cDNA production, or a single cycle of DNA replication. The polynucleotide can also be a population of amplified products (from a few to very many), for example single stranded DNA products produced using single primer isothermal amplification and/or Ribo-SPIA™,see Kurn, U.S. Pat. No. 6,251,639 B1; Kurn, U.S. Patent Publication No. 2003/0087251 A1, or double stranded DNA product produced by, for example, PCR. It is further understood that a polynucleotide comprising a non-canonical nucleotide can be a multiplicity (from small to very large) of different polynucleotide molecules. Such populations can be related in sequence (e.g., member of a gene family or superfamily) or extremely diverse in sequence (e.g., generated from all mRNA, generated from all genomic DNA, etc.). Polynucleotides can also correspond to single sequence (which can be part or all of a known gene, for example a coding region, genomic portion, etc.). Methods, reagents, and reaction conditions for generating specific polynucleotide sequences and multiplicities of polynucleotide sequences are known in the art.

Generating a Polynucleotide Comprising an Abasic Site

A polynucleotide comprising an abasic site can be generated using methods known in the art (e.g., Makrigiorgos, Int J. Radiat. Biol. (1998) 74(1):99-109), and as described herein. Generally, a polynucleotide comprising a non-canonical nucleotide (which can be synthesized from a template, as described herein) is treated with an enzyme capable of generally, specifically or selectively cleaving a base portion of the non-canonical nucleotide to create an abasic site. The embodiment shown in FIG. 3 illustrates cleavage of a base portion of the non-canonical nucleotides, whereby an abasic site is created. Generally, an agent, such as an enzyme, catalyzes hydrolysis of the bond between the base portion of the non-canonical nucleotide and a sugar in the non-canonical nucleotide to generate an abasic site comprising a hemiacetal ring and lacking the base (interchangeably called “AP” site), though other cleavage products are contemplated for use in the methods of the invention. Suitable agents and reaction conditions for cleavage of base portions of non-canonical nucleotides are known in the art and described herein.

Generally, cleavage of base portions of non-canonical nucleotides is general, specific or selective cleavage, whereby greater than about 98%, about 95%, about 90%, about 85%, or about 80% of the base portions cleaved are bases portions of non-canonical nucleotides. However, extent of cleavage can be less. Thus, reference to specific cleavage is exemplary. In embodiments involving generation of polynucleotide fragments, specific or selective cleavage is desirable for control of the fragment size in the methods of generating immobilized nucleotide fragments of the invention (i.e., the fragments generated by cleavage of the phosphodiester backbone at an abasic site). Generally, reaction conditions are selected such that the reaction in which the abasic site(s) are created can run to completion.

For convenience, the synthesis of a polynucleotide comprising a non-canonical nucleotide, and the cleavage of that polynucleotide by an enzyme capable of cleaving a base portion of the non-canonical nucleotide are described as separate steps. It is understood that these steps may be performed simultaneously, except (generally) in the case when a polynucleotide comprising a non-canonical nucleotide must be capable of serving as a template for further amplification (as in exponential methods of amplification, e.g. PCR).

Cleaving the Phosphodiester Backbone at the Abasic Site of the Polynucleotide Comprising an Abasic Site

In some embodiments, the phosphodiester backbone of the polynucleotide is cleaved at the abasic site with an agent capable of effecting cleavage of a backbone at the abasic site, whereby polynucleotide fragments are generated. The embodiment shown in FIG. 3 illustrates cleavage of the backbone immediately 5′ to the abasic sites of the polynucleotide comprising the abasic sites, whereby cleaved fragments are produced. Cleavage of the backbone at an abasic site is described herein. Suitable enzymes and/or reaction conditions for cleavage of the backbone are well known in the art, and are described herein.

As noted herein, the frequency of incorporation of non-canonical nucleotides into the polynucleotide relates to the size of fragment produced using the methods of the invention because the spacing between non-canonical nucleotides in the polynucleotide comprising a non-canonical nucleotide determines the approximate size of the resulting fragments (following generation of an abasic site from the non-canonical nucleotide and cleavage of the phosphodiester backbone at the site of incorporation of the non-canonical nucleotide (also termed the abasic site), as described herein). Generally, suitable fragment sizes are about 5, 10, 15, 20, 25, 30, 40, 50, 65, 75, 85, 100, 123, 150, 175, 200, 225, 250, 300, 350, 400,450, 500, 550, 600, 650 or more nucleotides in length. It is understood that the fragment size is approximate, particularly when populations of fragments are generated, because the incorporation of a non-canonical nucleotide (which relates to the fragment size following cleavage) will vary from template to template, and also between copies of the same template. Thus, fragments generated from same starting material may have different (and/or overlapping) sequence, while still having the same approximate size or size range.

Generally, cleavage of the backbone at an abasic site is general, specific or selective cleavage (in the sense that the agent (such as an enzyme) capable of cleaving the backbone at an abasic site specifically or selectively cleaves the base portion of a particular non-canonical nucleotide), whereby greater than about 98%, about 95%, about 90%, about 85%, or about 80% of the cleavage is at an abasic site. However, extent of cleavage can be less. Thus, reference to specific cleavage is exemplary. Generally, about 98%, about 95%, about 90%, about 85%, or about 80% of the abasic sites at the backbone are cleaved. However, extent of cleavage can be less (such that fragments comprising uncleaved abasic sites are produced). In some embodiments, abasic sites are labeled (either before or after immobilization to a substrate, as described herein).

Labeling at an Abasic Site

In some embodiments, the polynucleotide, or fragment thereof, is labeled at an abasic site. Labeling is as described herein. As the disclosure herein makes clear, it is understood that labeling and fragmentation steps or labeling and immobilization steps, or labeling and immobilization, and fragmentation steps, can be performed in any order, or simultaneously.

Immobilizing a Polynucleotide Comprising an Abasic Site to a Substrate

After generation of the polynucleotide comprising an abasic site, the polynucleotide (or polynucleotide fragment, if the backbone is cleaved) is immobilized to a substrate at the abasic site. In embodiments involving cleavage of the backbone at an abasic site (whereby fragments of the synthesized nucleic acid are generated), the cleaved fragments are immobilized to a substrate at the cleaved abasic site. FIG. 3 diagrammatically depicts an embodiment in which a polynucleotide fragment is immobilized to a substrate at the cleaved abasic site. Immobilizing a polynucleotide(s) is useful, for example, to tag an analyte, or to create a microarray. Single stranded polynucleotides (including polynucleotide fragments) are particularly suitable for preparing microarrays comprising the single stranded polynucleotides. Single stranded polynucleotide fragments (in embodiments involving cleavage of the phosphodiester backbone at an abasic site) are advantageous, because the orientation of the fragment with respect to the substrate (upon which the fragment is immobilized) can be controlled by selection of the method used to cleave the phosphodiester backbone, such that an abasic site is positioned at the 3′ end of a fragment or at the 5′ end of a fragment. Immobilizing polynucleotides in a defined orientation (e.g., at the 3′ end, at the 5′ end) enhances hybridization of complementary oligonucleotides, and permits a higher density of immobilization.

The polynucleotide comprising the abasic site is immobilized to a substrate as follows: generally, reagents are used that are capable of covalently or non-covalently attaching a reactive group present in the abasic site to a reactive group present on a substrate. For example, a common functional group exposed in an abasic site (and therefore suitable for use in labeling) is the aldehyde of the hemiacetal ring which can be covalently or noncovalently attached to a reactive group on a suitable substrate using reaction conditions that are known in the art. Suitable sidechains (present on the substrate) to react with the aldehyde (of the abasic site) include at least the following: substituted hydrazines, hydrazides, or hydroxylamines (which readily form imine bonds with aldehydes), and the related semicarbazide and thiosemicarbazide groups, and other amines which can form stable carbon-nitrogen double bonds, that can catalyze simultaneous cleavage and binding (see Horn, Nucl. Acids. Res., (1988) 16:11559-71), or can be coupled to form stable conjugates, e.g. by reductive amination.

The substrate to which the polynucleotide is to be immobilized can be functionalized with suitable reactive groups using methods known in the art. For example, a solid or semi-solid substrate (e.g., silicon or glass slide) can be coated with polymers (e.g., polyacrylamide, dextran, acrylamide, or latex) comprising hydrazine, hydrazide, or amine derivatized substrates (e.g. semicarbazides). Methods for functionalizing substrates with suitable reactive groups are known in the art, and disclosed in, for example, Luktanov, U.S. Pat. No. 6,339,147; Van Ness, U.S. Pat. No. 5,667,976; Bangs Laboratories, Inc. TechNote 205 (available at bangslabs.com); Ghosh, Anal. Biochem (1989) 178:43-51; O'Shannessy, Anal. Biochem. (1990) 191:1-8; Wilchek, Methods Enzymol. (1987) 138:429-442; Baumgartner, Anal. Biochem. (1989) 181:182-189; Zalipsky, Bioconjugate Chem. (1995) 6: 150-165, and references cited therein.

Methods and reaction conditions for performing these reactions are known in the art. See, e.g. Luktanov, U.S. Pat. No. 6,339,147; Van Ness, U.S. Pat. No. 5,667,976; Bangs Laboratories, Inc. TechNote 205 (available at bangslabs.com); Ghosh, Anal. Biochem (1989) 178:43-51; O'Shannessy, Anal. Biochem. (1990) 191:1-8; Wilchek, Methods Enzymol. (1987) 138:429-442; Baumgartner, Anal. Biochem. (1989) 181:182-189; Zalipsky, Bioconjugate Chem. (1995) 6: 150-165, and references cited therein. It is appreciated that similar chemistry is described herein with respect to the methods of labeling an abasic site (i.e., embodiments in which a reactive group in the abasic site is covalently or non-covalently attached to a suitable reactive group on a label). See, e.g., Srivastava, J. Biol. Chem. (1998) 273(33): 21203-209; Makrigiorgos, Int J Radiat. Biol. (1998) 74(1):99-109; Makriogiorgos, U.S. Pat. No. 6,174,680 B1; Makrogiorgos, WO 00/39345.

In another example, the abasic site may be chemically modified, then the modified abasic site covalently or non-covalently attached to a suitable reactive group on a substrate. For example, the aldehyde (in the abasic site) can be oxidized or reduced (using methods known in the art), then covalently immobilized to a substrate using, e.g., reductive amination or various oxidative processes.

The substrate may consist of many materials, limited primarily by capacity to immobilize (or, in some embodiments, capacity for derivatization to immobilize) any of a number of chemically reactive groups and compatibility with the synthetic chemistry used to immobilize the polynucleotide comprising an abasic site. The substrate can be a solid or semi-solid support, which may be made, e.g., from glass, plastic (e.g., polystyrene, polypropylene, nylon), polyacrylamide, nitrocellulose, or other materials such as metals. As described herein, the substrate can be functionalized, if necessary to add a suitable reactive group (to which the abasic site is covalently or non-covalently immobilized). The polynucleotides may also be spotted as a matrix on substrates comprising paper, glass, plastic, polystyrene, polypropylene, nylon, polyacrylamide, nitrocellulose, silicon, optical fiber or any other suitable solid or semi-solid (e.g., thin layer of polyacrylamide gel, assuming that the substrate is suitably functionalized, as described herein (Khrapko, et al., DNA Sequence (1991), 1:375-388)).

An array may be assembled as a two-dimensional matrix on a planar substrate or may have a three-dimensional configuration comprising pins, rods, fibers, tapes, threads, beads, particles, microtiter wells, capillaries, cylinders and any other arrangement suitable for hybridization and detection of template molecules. In one embodiment the substrate to which the polynucleotide (or fragments thereof) is immobilized is magnetic beads or particles. In another embodiment, the solid substrate comprises an optical fiber. In yet another embodiment, the polynucleotides are dispersed in fluid phase within a capillary which, in turn, is immobilized with respect to a solid phase.

In another embodiment, the substrate comprises a polypeptide, a protein, a peptide, carbohydrates, cells, microorganisms and fragments and products thereof, an organic molecule, an inorganic molecule, carrier molecules, PEG, aminodextran, carbohydrates, supramolecular assemblies, organelles, cells, microorganisms, organic molecules, inorganic molecules, or any substance for which immobilization sites for polynucleotides comprising abasic sites naturally exist, can be created (e.g. by functionalizing or otherwise modifying the substrate) or can be developed. In one embodiment, the substrate is a polynucleotide.

The substrate may be an analyte. Typical analytes may include, but are not limited to antibodies, proteins (including enzymes), peptides, nucleic acid molecules or segments thereof, carrier molecules, PEG, amino-dextran, carbohydrates, supramolecular assemblies, organelles, cells, microorganisms, organic molecules, inorganic molecules, or any substance for which immobilization sites for polynucleotides comprising abasic sites naturally exist, can be created (e.g. by functionalizing the analyte) or can be developed.

It is understood that a substrate may be a member(s) of a binding pair. Non-limiting examples of a binding pair include a protein:protein binding pair, and a protein: antibody binding pair. In another embodiment, polynucleotides (or fragments thereof) are immobilized to (tag) a molecular library of substrates, e.g., a molecular library of chemical compounds, a phage peptide display library, or a library of antibodies.

In some embodiments, the substrate (to which the polynucleotide is immobilized) is an enzyme, such that enhanced detection of hybridization of the polynucleotide is provided. For example, a polynucleotide immobilized to an enzyme can be hybridized to a microarray, and hybridized polynucleotide detected by contacting the microarray with a defined substrate.

In embodiments of the invention involving cleavage of the phosphodiester backbone at an abasic site (whereby fragments of the synthesized nucleic acid are generated), the cleaved fragments can also be immobilized to a substrate using any method known in the art for immobilization of a nucleic acid to a substrate.

For example, single or double stranded polynucleotide fragments (generally single stranded) can be immobilized to a solid or semi-solid support or substrate, which may be made, e.g., from plastics, ceramics, metals, acrylamide, cellulose, nitrocellulose, glass, polystyrene, polyethylene vinyl acetate, polypropylene, polymethacrylate, polyethylene, polyethylene oxide, polysilicates, polycarbonates, Teflon®, fluorocarbons, nylon, silicon rubber, polyanhydrides, polyglycolic acid, polylactic acid, polyorthoesters, polypropylfumerate, collagen, glycosaminoglycans, and polyamino acids, and other materials. Substrates may be two-dimensional or three-dimensional in form, such as gels, membranes, thin films, glasses, plates, cylinders, beads, magnetic beads, optical fibers, woven fibers, microtiter well, capillaries, etc. For example, the fragments can be contacted with a solid or semi-solid substrate, such as a glass slide, which is coated with a reactive group which will form a covalent link with the reactive group that is on the polynucleotide fragment and become covalently immobilized to the substrate.

Microarrays comprising the nucleotide fragments can be fabricated using a Biodot (BioDot, Inc. Irvine, Calif.) spotting apparatus and aldehyde-coated glass slides (CEL Associates, Houston, Tex.). Polynucleotide fragments can be spotted onto the aldehyde-coated slides following suitable functionalization, and processed according to published procedures (Schena et al., Proc. Natl. Acad. Sci. U.S.A. (1995) 93:10614-10619), provided suitable care is taken to avoid interfering with other desired reactions at the abasic sites. Arrays can also be printed by robotics onto glass, nylon (Ramsay, G., Nature Biotechnol. (1998), 16:40-44), polypropylene (Matson, et al., Anal Biochem. (1995), 224(1):110-6), and silicone slides (Marshall, A. and Hodgson, J., Nature Biotechnol. (1998), 16:27-31). Other approaches to array assembly include fine micropipetting within electric fields (Marshall and Hodgson, supra), and spotting the polynucleotides directly onto positively coated plates. Methods such as those using amino propyl silane surface chemistry are also known in the art, as disclosed at http://www.cmt.corning.com and http://cmgm.stanford.edu/pbrown/.

One method for making microarrays is by making high-density polynucleotide arrays. Techniques are known for rapid deposition of polynucleotides (Blanchard et al., Biosensors & Bioelectronics, 11:687-690). In principle, and as noted above, any type of array, for example, dot blots on a nylon hybridization membrane, could be used. However, as will be recognized by those skilled in the art, very small arrays will frequently be preferred because hybridization volumes will be smaller.

Methods for immobilizing polynucleotide fragments to analytes (as described herein) are known in the art. See, e.g., U.S. Pat. Nos. 6,309,843; 6,306,365; 6,280,935; 6,087,103 (and methods discussed therein).

It is understood that the polynucleotide fragments prepared according to the method of the invention can comprise a free 3′-hydroxyl or a free 5′-hydroxyl group. Methods and reaction conditions for immobilization of nucleotide through free hydroxyl groups are well known in the art. See, e.g., U.S. Pat. Nos. 6,169,194; 5,726,329.

Reaction Conditions and Detection

Appropriate reaction media and conditions for carrying out the methods of the invention are those that permit nucleic acid synthesis according to the methods of the invention. Such media and conditions are known to persons of skill in the art, and are described in various publications, such as U.S. Pat. Nos. 6,190,865; 5,554,516; 5,716,785; 5,130,238; 5,194,370; 6,090,591; 5,409,818; 5,554,517; 5,169,766; 5,480,784; 5,399,491; 5,679,512; PCT Pub. No. WO99/42618; Mol. Cell Probes (1992) 251-6; and Anal. Biochem. (1993) 211:164-9. For example, a buffer may be Tris buffer, although other buffers can also be used as long as the buffer components are non-inhibitory to enzyme components of the methods of the invention. The pH is preferably from about 5 to about 11, more preferably from about 6 to about 10, even more preferably from about 7 to about 9, and most preferably from about 7.5 to about 8.5. The reaction medium can also include bivalent metal ions such as Mg²⁺ or Mn²⁺, at a final concentration of free ions that is within the range of from about 0.01 to about 15 mM, and most preferably from about 1 to 10 mM. The reaction medium can also include other salts, such as KCl or NaCl, that contribute to the total ionic strength of the medium. For example, the range of a salt such as KCl is preferably from about 0 to about 125 mM, more preferably from about 0 to about 100 mM, and most preferably from about 0 to about 75 mM. The reaction medium can further include additives that could affect performance of the amplification reactions, but that are not integral to the activity of the enzyme components of the methods. Such additives include proteins such as BSA, single strand binding proteins (e.g., T4 gene 32 protein), and non-ionic detergents such as NP40 or Triton. Reagents, such as DTT, that are capable of maintaining enzyme activities can also be included. Such reagents are known in the art. Where appropriate, an-RNase inhibitor (such as Rnasin) that does not inhibit the activity of the RNase employed in the method (if any) can also be included. Any aspect of the methods of the invention can occur at the same or varying temperatures. The synthesis reactions (particularly, primer extension other than the first and second strand cDNA synthesis steps, and strand displacement) can be performed isothermally, which avoids the cumbersome thermocycling process. The synthesis reaction is carried out at a temperature that permits hybridization of the oligonucleotides (primer) of the invention to the template polynucleotide and primer extension products, and that does not substantially inhibit the activity of the enzymes employed. The temperature can be in the range of preferably about 25° C. to about 85° C., more preferably about 30° C. to about 80° C., and most preferably about 37° C. to about 75° C. In some embodiments that include RNA transcription, the temperature for the transcription steps is lower than the temperature(s) for the preceding steps. In these embodiments, the temperature of the transcription steps can be in the range of preferably about 25° C. to about 85° C., more preferably about 30° C. to about 75° C., and most preferably about 37° C. to about 70° C.

Nucleotides, including non-canonical nucleotides (or other nucleotide analogs), that can be employed for synthesis of the nucleic acid comprising a non-canonical nucleotide in the methods of the invention are provided in the amount of from preferably about 50 to about 2500 μM, more preferably about 100 to about 2000 μM, even more preferably about 200 to about 1700 μM, and most preferably about 250 to about 1500 μM. The oligonucleotide components of the synthesis reactions of the invention are generally in excess of the number of template nucleic acid sequence to be replicated. They can be provided at about or at least about any of the following: 10, 10², 10⁴, 10⁶, 10⁸, 10¹² times the amount of target nucleic acid. Composite primers can be provided at about or at least about any of the following concentrations: 50 nM, 100 nM, 500 nM, 1000 nM, 2500 nM, 5000 nM.

Optionally, the polynucleotide comprising a non-canonical nucleotide can be treated with hydroxylamine (or any other suitable agent) to remove any aldehydes that may have formed spontaneously in the nucleic acid. See, e.g., Makrogiorgos, WO00/39345.

For convenience, the synthesis of a polynucleotide comprising a non-canonical nucleotide, and the cleavage of a base portion of that polynucleotide by an enzyme capable of cleaving a base portion of the non-canonical nucleotide, and the cleavage of the phosphodiester backbone at the abasic site, are described as separate steps. It is understood that these steps may be performed simultaneously, except (generally) in the case when a polynucleotide comprising a non-canonical nucleotide must be capable of serving as a template for further amplification (as in exponential methods of amplification, e.g. PCR).

Appropriate reaction media and conditions for carrying out the cleavage of a base portion of a non-canonical nucleotide according to the methods of the invention are those that permit cleavage of a base portion of a non-canonical nucleotide. Such media and conditions are known to persons of skill in the art, and are described in various publications, such as Lindahl, PNAS (1974) 71(9):3649-3653; Jendrisak, U.S. Pat. No. 6,190,865 BI; U.S. Pat. No. 5,035,996; U.S. Pat. No. 5,418,149. For example, buffer conditions can be as described above with respect to polynucleotide synthesis. In one embodiment, UDG (Epicentre Technologies, Madison Wis.) is added to a nucleic acid synthesis reaction mixture, and incubated at 37° C. for 20 minutes. In one embodiment, the reaction conditions are the same for the synthesis of a polynucleotide comprising a non-canonical nucleotide and the cleavage of a base portion of the non-canonical nucleotide. In another embodiment, different reaction conditions are used for these reactions. In some embodiments, a chelating regent (e.g. EDTA) is added before or concurrently with UNG in order to prevent the polymerase from extending the ends of the cleavage products.

In embodiments involving cleavage of the phosphodiester backbone, appropriate reaction media and conditions for carrying out the cleavage of the phosphodiester backbone at an abasic site according to the methods of the invention are those that permit cleavage of the phosphodiester backbone at an abasic site. Such media and conditions are known to persons of skill in the art, and are described in various publications, such as Bioorgan. Med. Chem (1991) 7:2351; Sugiyama, Chem. Res. Toxicol. (1994) 7: 673-83; Horn, Nucl. Acids. Res., (1988) 16:11559-71); Lindahl, PNAS (1974) 71(9):3649-3653; Jendrisak, U.S. Pat. No. 6,190,865 B1; Shida, Nucleic Acids Res. (1996) 24(22):4572-76; Srivastava, J. Biol Chem. (1998) 273(13):21203-209; Carey, Biochem. (1999) 38:16553-60; Chem Res Toxicol (1994) 7:673-683. For example, E. coli AP endonuclease IV is added to reaction conditions as described above. AP Endonuclease IV can be added at the same or different time as the agent (such as an enzyme) capable of cleaving the base portion of a non-canonical nucleotide. For example, AP Endonuclease IV can be added at the same time as UNG, or at different times. A reaction mixture suitable for simultaneous UNG treatment and N,N′-dimethylethylenediamine treatment is described in Example 4 herein.

In another example, nucleic acids containing abasic sites are heated in a buffer solution containing an amine, for example, 25 mM Tris-HCl and 1-5 mM magnesium ions, for 10-30 minutes at 70° C. to 95° C. Alternatively, 1.0 M piperidine (a base) is added to polynucleotide comprising an abasic site which has been precipitated with ethanol and vacuum dried. The solution is then heated for 30 minutes at 90° C. and lyophilized to remove the piperidine. In another example, cleavage is effected by treatment with basic solution, e.g., 0.2 M sodium hydroxide at 37° for 15 minutes. See Nakamura (1998) Cancer Res. 58:222-225. In yet another example, incubation at 37° C. with 100 mM N,N′-dimethylethylenediamine acetate, pH 7.4 is used to cleave. See McHugh and Knowland, (1995) Nucl. Acids Res. 23(10) 1664-1670.

In one embodiment, the reaction conditions are the same for the cleavage of a base portion of the non-canonical nucleotide and for the cleavage of the phosphodiester backbone at abasic sites. In another embodiment, different reaction conditions are used for these reactions.

In embodiments involving labeling at an abasic site, appropriate reaction media and conditions for carrying out the labeling at an abasic site according to the methods of the invention are those that permit labeling at an abasic site. Such reaction mixtures and conditions are known to persons of skill in the art, and are described in various publications, such as Makrogiorgos, WO 00/39345; Srivastava, J. Biol. Chem. (1998) 273(33): 21203-209; Makrigiorgos,Int J. Radiat. Biol. (1998) 74(1):99-109; Makriogiorgos, U.S. Pat. No. 6,174,680 B1; Makrogiorgos, WO 00/39345; Boturyn (1999) Chem. Res. Toxicol. 12:476-482. See, also, Adamczyk (1998) Bioorg. Med. Chem. Lett. 8(24):3599-3602; Adamczyk (1999) Org. Lett. 1(5):779-781; Kow (2000) Methods 22(2):164-169; Molecular Probes Handbook, Section 3.2 (www.probes.com); Horn (Nucl. Acids. Res., (1988) 16:11559-71). For example, 5-(((2-(carbohydrazino)-methyl)thio)acetyl)aminofluorescein, aminooxyacetyl hydrazide (FARP); N-(aminooxyacetyl)-N′-(D-biotinoyl) hydrazine, trifluorecetic acid salt (ARP); Alexa Fluor 555 (Molecular Probes); aminooxy-derivatized Alexa Fluor 555; and other aldehyde-reactive reagents can be reacted with a polynucleotide comprising abasic sites. The buffer can be sodium citrate or sodium phosphate buffer, though other buffers are acceptable as long as the buffer components are non-inhibitory to enzyme components and/or desired chemical reactions used in the methods of the invention. The pH is preferably from about 3 to about 11, more preferably from about 4 to about 10, even more preferably from about 4 to about 8, and most preferably from about 4 to about 7. The reaction can be conducted at room temperature to 37°, though other temperatures are suitable as long as the temperature is non-inhibitory to enzyme components and/or desired chemical reactions used in the methods of the invention. Generally, the label (e.g. ARP or FARP) is added at about 1-10 mM, preferable 2-5 mM, though other concentrations are suitable. If an antibody label is used, conditions for antibody binding are well-known in the art, and can be as described herein. Optionally, a stop buffer can be used that neutralizes the pH of the labeling reaction, thereby stopping the labeling reaction and optionally, facilitating subsequent purification of labeled product.

In embodiments involving immobilization of a polynucleotide at an abasic site, appropriate reaction media and conditions for carrying out the immobilization at an abasic site according to the methods of the invention are those that permit immobilization at an abasic site. Such reaction mixtures and conditions are known to persons of skill in the art, and are described in various publications, such as Luktanov, U.S. Pat. No. 6,339,147; Van Ness, U.S. Pat. No. 5,667,976; Bangs Laboratories, Inc. TechNote 205 (available at bangslabs.com); Ghosh, Anal. Biochem (1989) 178:43-51; O'Shannessy, Anal. Biochem. (1990) 191:1-8; Wilchek, Methods Enzymol. (1987) 138:429-442; Baumgartner, Anal. Biochem. (1989) 181:182-189; Zalipsky, Bioconjugate Chem. (1995) 6: 150-165, and references cited therein. In some cases, the initial product can be stabilized by reduction with sodium cyanoborohydride or similar agents known in the art. See, e.g., O'Shannessy, supra.

In one embodiment, the foregoing components are added simultaneously at the initiation of the synthesis step of the fragmentation and/or labeling and/or immobilization processes. In another embodiment, components are added in any order prior to or after appropriate timepoints during the synthesis step. Such timepoints, some of which are noted below, can be readily identified by a person of skill in the art. In these embodiments, the reaction conditions and components may be varied between the different reactions.

The fragmenting and/or labeling and/or immobilization process can be stopped at various timepoints, and resumed at a later time. Said timepoints can be readily identified by a person of skill in the art. Methods for stopping the reactions are known in the art, including, for example, cooling the reaction mixture to a temperature that inhibits enzyme activity or heating the reaction mixture to a temperature that destroys an enzyme. Methods for resuming the reactions are also known in the art, including, for example, raising the temperature of the reaction mixture to a temperature that permits enzyme activity or replenishing a destroyed (depleted) enzyme or other reagent. In some embodiments, one or more of the components of the reactions is replenished prior to, at, or following the resumption of the reactions. Alternatively, the reaction can be allowed to proceed (i.e., from start to finish) without interruption.

The reaction can be allowed to proceed without purification of intermediate complexes, for example, to remove primer. Products can be purified at various timepoints, which can be readily identified by a person of skill in the art.

Compositions and Kits of the Invention

The invention also provides compositions and kits used in the methods described herein. The compositions may be any component(s), reaction mixture and/or intermediate described herein, as well as any combination. For example, the invention provides a composition comprising a primer (which can be an RNA-DNA composite primer), non-canonical nucleotides, an agent (such as an enzyme) capable of cleaving a base portion of a non-canonical nucleotide, optionally an agent (such as an enzyme) capable of effecting cleavage of a phosphodiester backbone at an abasic site, and an agent capable of labeling an abasic site. In another example, the invention provides a composition comprising a polynucleotide comprising a non-canonical nucleotide, said polynucleotide synthesized from a template, and an agent capable of labeling an abasic site. In still another example, the composition comprises a primer (which can be a RNA-DNA composite primer), dUTP, UNG, (optionally) E. coli Endonuclease IV, and N-(aminooxyacetyl)-N′-(D-biotinoyl) hydrazine, trifluorecetic acid salt (ARP).

In another embodiment, the invention provides a composition comprising a composite primer, said composite comprising a DNA portion and a 5′ RNA portion; and a non-canonical nucleotide (such as dUTP). In another embodiment, the composition comprises a composite primer, said composite primer comprising an RNA portion and a 3′ DNA portion; and an agent (such as UNG) that is capable of cleaving a base portion from a non-canonical nucleotide. In another embodiment, the composition comprises a composite primer, said composite primer comprising an RNA portion and a 3′ DNA portion; and an agent (such as an amine, such as N,N′-dimethylethylenediamine) capable of cleaving the phosphodiester back at an abasic site. In other embodiments, the composition comprises a composite primer, said composite primer comprising an RNA portion and a 3′ DNA portion; and an agent that labels an abasic site (such as ARP). In other embodiments, the composition comprises a composite primer, said composite primer comprising an RNA portion and a 3′ DNA portion; dUTP; and UNG. In still other embodiments, the composition comprises a composite primer, said composite primer comprising an RNA portion and a 3′ DNA portion; dUTP; UNG; and ARP. In still other embodiments, the composition comprises a composite primer, said composite primer comprising an RNA portion and a 3′ DNA portion; dUTP; UNG; and N,N′-dimethylethylenediamine. In still other embodiments, the composition comprises a composite primer, said composite primer comprising an RNA portion and a 3′ DNA portion; dUTP; UNG; N,N′-dimethylethylenediamine; and ARP.

In still other embodiments, the invention provides a composition comprising a composite primer, said composite primer comprising an RNA portion and a 3′ DNA portion; a non-canonical nucleotide; an agent (such as an enzyme) capable of cleaving a base portion of a non-canonical nucleotide; an agent (such as an enzyme) capable of cleaving a phosphodiester backbone at an abasic site; and an agent capable of labeling an abasic site. In some embodiment, the composition further provides a suitable substrate for immobilization. In some embodiments, the RNA portion is 5′ to the DNA portion, the 5′ RNA portion of the composite primer is adjacent to the 3′ DNA portion, the RNA portion of the composite primer consists of about 10 to about 20 nucleotides and the DNA portion of the composite primer consists of about 7 to about 20 nucleotides. In still other embodiments, the composition comprises a second, different composite primer. In some embodiments, the RNA portion of the composite primer comprises the following ribonucleotide sequence: 5′-GACGGAUGCGGUCU-3′.

In still another embodiment, invention provides a composition comprising (a) UNG; (b) N,N′-dimethylethylenediamine; and (c) ARP. In other embodiments, the invention provides a composition comprising (a) UNG; (b) N,N′-dimethylethylenediamine; (c) ARP; (d) dUTP; (e) a mixture of dATP, dTTP, dCTP, and dGTP; (f) a DNA polymerase; (g) a composite primer, wherein the composite primer comprises a 5′ RNA portion and a 3′ DNA portion. In still other embodiments, the invention provides a composition comprising (a) UNG; (b) N,N′-dimethylethylenediamine; (c) ARP; (d) dUTP; (e) a mixture of dATP, dTTP, dCTP, and dGTP; (f) a DNA polymerase; (g) RNAse H; (h) a composite primer, wherein the composite primer comprises a 5′ RNA portion and a 3′ DNA portion. In yet another embodiment, the invention provides a composition comprising (a) UNG; (b) N,N′-dimethylethylenediamine; (c) ARP; (d) dUTP; (e) a mixture of dATP, dTTP, dCTP, and dGTP; (f) a DNA polymerase; (g) RNAse H; (h) a composite primer, wherein the composite primer comprises a 5′ RNA portion and a 3′ DNA portion (i) MgCl₂ solution; (j) acetic acid solution; and optionally, (k) a stop buffer comprising 1.5M Tris, pH 8.5. In some embodiments, the dUTP and the mixture of dATP, dTTP, dCTP, and cGTP are combined. In some embodiments, the DNA polymerase and RNAse H are provided as a mixture. In some embodiments, the RNA portion of the composite primer is 5′ with respect to the 3′ DNA portion, the 5′ RNA portion is adjacent to the 3′ DNA portion, the RNA portion of the composite primer consists of about 10 to about 20 nucleotides and the DNA portion of the composite primer consists of about 7 to about 20 nucleotides. In still other embodiments, the composition comprises a second, different composite primer. In some embodiments, the RNA portion of the composite primer comprises the following ribonucleotide sequence: 5′-GACGGAUGCGGUCU-3′.

In still other embodiments, the invention provides a composition comprising: (a) UNG; (b) ARP; (c) dUTP; (d) a DNA polymerase; (e) RNAse H; (f) a composite primer, wherein the composite primer comprises a 5′ RNA portion and a 3′ DNA portion. In other embodiments, the composition further comprises (g) MgCl₂ solution; (h) acetic acid solution; and optionally, (i) a stop buffer comprising 1.5M Tris, pH 8.5. In other embodiments, the invention provides a composition comprising (a) UNG; (b) an agent capable of labeling an abasic site (for example, Alexa Fluor 555 or an aminooxy-modified Alexa Fluor 555); (c) dUTP; (d) a DNA polymerase; (e) RNAse H; (f) a composite primer, wherein the composite primer comprises a 5′ RNA portion and a 3′ DNA portion. In some embodiments, the DNA polymerase and RNAse H are provided as a mixture. In some embodiments, the RNA portion of the composite primer is 5′ with respect to the 3′ DNA portion, the 5′ RNA portion is adjacent to the 3′ DNA portion, the RNA portion of the composite primer consists of about 10 to about 20 nucleotides and the DNA portion of the composite primer consists of about 7 to about 20 nucleotides. In still other embodiments, the composition comprises a second, different composite primer. In some embodiments, the RNA portion of the composite primer comprises the following ribonucleotide sequence: 5′-GACGGAUGCGGUCU-3′.

In another example, the invention provides compositions comprising a polynucleotide comprising an abasic site and a suitable substrate for attachment through an abasic site (e.g., a microarray; an analyte), which may be functionalized if necessary. In still another example, the invention provides a composition comprising a polynucleotide comprising a non-canonical nucleotide, UNG, (optionally) E. coli Endonuclease IV, and a suitable substrate for attachment through an abasic site, which may be functionalized if necessary.

The compositions are generally in lyophilized or aqueous form (if appropriate), preferably in a suitable buffer.

The invention also provides compositions comprising the labeled and/or fragmented products described herein. Accordingly, the invention provides a population of labeled and/or fragmented polynucleotides, which are produced by any of the methods described herein (or compositions comprising the products).

The invention also provides compositions comprising the immobilized polynucleotides or immobilized polynucleotide fragments described herein. In some embodiments, the immobilized polynucleotide (or immobilized fragment, in embodiments involving fragmentation) are labeled, as described herein. Accordingly, the invention provides a population of immobilized polynucleotides or immobilized polynucleotide fragments which are produced by any of the methods described herein (or compositions comprising the products).

The compositions are generally in a suitable medium, although they can be in lyophilized form. Suitable media include, but are not limited to, aqueous media (such as pure water or buffers).

The invention also provides reaction mixtures (or compositions comprising reaction mixtures) which contain various combinations of components described herein. Examples of reaction mixtures have been described. In some embodiments, the invention provides reaction mixtures comprising: a composite primer, said composite primer comprising an RNA portion and a 3′ DNA portion; and a non-canonical nucleotide (such as dUTP). In another embodiment, the reaction mixture comprises a polynucleotide comprising an abasic site, wherein the polynucleotide was synthesized using a composite primer; and an agent (such as UNG) that is capable of cleaving a base portion from a non-canonical nucleotide. In another embodiment, the reaction mixture comprises a polynucleotide comprising an abasic site, wherein the polynucleotide was synthesized using a composite primer; and an agent (such as an amine, such as N,N′-dimethylethylenediamine) capable of cleaving the phosphodiester back at an abasic site. In other embodiments, the reaction mixture comprises a polynucleotide comprising an abasic site, wherein the polynucleotide was synthesized using a composite primer; and an agent that labels an abasic site (such as ARP). In other embodiments, the reaction mixture comprises a composite primer, said composite primer comprising an RNA portion and a 3′ DNA portion; dUTP; and UNG. In still other embodiments, the reaction mixture comprises a polynucleotide comprising an abasic site, wherein the polynucleotide was synthesized using a composite primer; and ARP. In still other embodiments, the reaction mixture comprises a polynucleotide comprising an abasic site, wherein the polynucleotide was synthesized using a composite primer; and N,N′-dimethylethylenediamine. In still other embodiments, the reaction mixture comprises a polynucleotide comprising an abasic site, wherein the polynucleotide was synthesized using a composite primer; N,N′-dimethylethylenediamine; and ARP. In still another embodiment, invention provides a reaction mixture comprising (a) UNG; (b) N,N′-dimethylethylenediamine; and (c) ARP. In other embodiments, the invention provides a reaction mixture comprising (a) UNG; and (b) N,N′-dimethylethylenediamine. In still other embodiments, the invention provides a reaction mixture comprising: (a) dUTP; (b) a DNA polymerase; (c) RNAse H; and (d) a composite primer, wherein the composite primer comprises a 5′ RNA portion and a 3′ DNA portion. In still other embodiments, the invention provides a reaction mixture comprising (a) a composite primer, wherein the composite primer comprises an RNA portion and a 3′ DNA portion; (b) dUTP; (c) a mixture of dATP, dTTP, dCTP, and dGTP; (d) a DNA polymerase; and (e) RNAse H. In still other embodiments, the invention provides a reaction mixture comprising a composite primer, said composite primer comprising an RNA portion and a 3′ DNA portion; and a non-canonical nucleotide. In some embodiment, the reaction mixture further provides a suitable substrate for immobilization. In some embodiments, the 5′ RNA portion of the composite primer is adjacent to the 3′ DNA portion, the RNA portion of the composite primer consists of about 10 to about 20 nucleotides and the DNA portion of the composite primer consists of about 7 to about 20 nucleotides. In still other embodiments, the reaction mixture comprises a second, different composite primer. In some embodiments, the RNA portion of the composite primer comprises the following ribonucleotide sequence: 5′-GACGGAUGCGGUCU-3′.

In other embodiments, the reaction mixture comprises a polynucleotide comprising an abasic site, wherein the polynucleotide was synthesized using a composite primer, said composite primer comprising an RNA portion and a 3′ DNA portion; and a substrate suitable for immobilization.

The invention provides kits for carrying out the methods of the invention. Accordingly, a variety of kits are provided in suitable packaging. The kits may be used for any one or more of the uses described herein, and, accordingly, may contain instructions for any one or more of the following uses: methods of producing a hybridization probe, characterizing and/or quantitating nucleic acid, detecting a mutation, preparing a subtractive hybridization probe, detection (using a hybridization probe), and determining a gene expression profile, using the labeled and/or fragmented nucleic acids generated by the methods of the invention.

The kits of the invention comprise one or more containers comprising any combination of the components described herein, and the following are examples of such kits. A kit may comprise: a primer (such as a RNA-DNA composite primer), a non-canonical nucleotide, an agent (such as an enzyme) capable of cleaving a base portion of a non-canonical nucleotide, an agent (such as an enzyme) capable of effecting cleavage of a phosphodiester backbone at an abasic site, and an agent capable of labeling an abasic site, which may or may not be separately packaged. In still another example, the kit comprises a primer (such as a composite primer as described herein), dUTP, UNG, (optionally) E. coli Endonuclease IV, and N-(aminooxyacetyl)-N′-(D-biotinoyl) hydrazine, trifluorecetic acid salt (ARP). In another embodiment, the kit comprises a primer (such as a composite primer), a non-canonical nucleotide, an agent (such as an enzyme) capable of cleaving a base portion of a non-canonical nucleotide, and an agent (such as an enzyme) capable of effecting cleavage of a phosphodiester backbone at an abasic site. In another embodiment, the kit comprises a polynucleotide comprising an abasic site, wherein the polynucleotide was generated by synthesis using a template, and an agent capable of labeling an abasic site. In still another example, the kit comprises a polynucleotide comprising a non-canonical nucleotide, UNG, (optionally) E. coli Endonuclease IV, and a suitable substrate for attachment through an abasic site (e.g. a microarray,; an analyte), which may be functionalized if necessary. In another embodiment, the kit comprises a polynucleotide comprising an abasic site and a suitable substrate (which may be functionalized if necessary) for attachment to an abasic site.

In other embodiments, the invention provides a kit comprising a primer (which can be an RNA-DNA composite primer), non-canonical nucleotides, an agent (such as an enzyme) capable of cleaving a base portion of a non-canonical nucleotide, optionally an agent (such as an enzyme) capable of effecting cleavage of a phosphodiester backbone at an abasic site, and an agent capable of labeling an abasic site. In another example, the invention provides a kit comprising a polynucleotide comprising a non-canonical nucleotide, said polynucleotide synthesized from a template, and an agent capable of labeling an abasic site. In still another example, the composition comprises a primer (which can be a RNA-DNA composite primer), dUTP, UNG, (optionally) E. coli Endonuclease IV, and N-(aminooxyacetyl)-N′-(D-biotinoyl) hydrazine, trifluorecetic acid salt (ARP).

In another embodiment, the invention provides a kit comprising a composite primer, said composite primer comprising an RNA portion and a 3′ DNA portion; and a non-canonical nucleotide (such as dUTP). In another embodiment, the composition comprises a composite primer, said composite primer comprising an RNA portion and a 3′ DNA portion; and an agent (such as UNG) that is capable of cleaving a base portion from a non-canonical nucleotide. In another embodiment, the kit comprises a composite primer, said composite primer comprising an RNA portion and a 3′ DNA portion; and an agent (such as an amine, such as N,N′-dimethylethylenediamine) capable of cleaving the phosphodiester back at an abasic site. In other embodiments, the kit comprises a composite primer, said composite primer comprising an RNA portion and a 3′ DNA portion; and an agent that labels an abasic site (such as ARP). In other embodiments, the kit comprises a composite primer, said composite primer comprising an RNA portion and a 3′ DNA portion; dUTP; and UNG. In still other embodiments, the kit comprises a composite primer, said composite primer comprising an RNA portion and a 3′ DNA portion; dUTP; UNG; and ARP. In still other embodiments, the kit comprises a composite primer, said composite primer comprising an RNA portion and a 3′ DNA portion; dUTP; UNG; and N,N′-dimethylethylenediamine. In still other embodiments, the kit comprises a composite primer, said composite primer comprising an RNA portion and a 3′ DNA portion; dUTP; UNG; N,N′-dimethylethylenediamine; and ARP.

In still other embodiments, the kit comprises an agent capable of cleaving RNA from a RNA-DNA hybrid (such as RNAse H); a non-canonical nucleotide (dUTP); and an agent capable of cleaving a base portion of a non-canonical nucleotide (UNG). In still other embodiments, the kit comprises an agent capable of cleaving RNA from a RNA-DNA hybrid (such as RNAse H); and an agent capable of labeling an abasic site (such as ARP, Alexa Fluor 555 hydrazide, or FARP). In still other embodiments, the kit comprises an agent capable of cleaving RNA from a RNA-DNA hybrid (such as RNAse H); and an agent capable of cleaving the backbone at an abasic site (such as an amine, such as N,N′-dimethylethylenediamine). In still other embodiments, the kit comprises RNAse H; N,N′-dimethylethylenediamine; and ARP.

In still other embodiments, the invention provides a kit comprising: a composite primer, said composite primer comprising an RNA portion and a 3′ DNA portion; a non-canonical nucleotide; an agent (such as an enzyme) capable of cleaving a base portion of a non-canonical nucleotide; an agent (such as an enzyme) capable of cleaving a phosphodiester backbone at an abasic site; and an agent capable of labeling an abasic site. In some embodiment, the kit further provides a suitable substrate for immobilization. In some embodiments, the 5′ RNA portion of the composite primer is adjacent to the 3′ DNA portion, the RNA portion of the composite primer consists of about 10 to about 20 nucleotides and the DNA portion of the composite primer consists of about 7 to about 20 nucleotides. In still other embodiments, the kit comprises a second, different composite primer. In some embodiments, the RNA portion of the composite primer comprises the following ribonucleotide sequence: 5′-GACGGAUGCGGUCU-3′.

In still another embodiment, invention provides a kit comprising (a) UNG; (b) N,N′-dimethylethylenediamine; and (c) ARP. In other embodiments, the invention provides a kit comprising (a) UNG; (b) N,N′-dimethylethylenediamine; (c) ARP; (d) dUTP; (e) a mixture of dATP, dTTP, dCTP, and dGTP; (f) a DNA polymerase; (g) a composite primer, wherein the composite primer comprises a 5′ RNA portion and a 3′ DNA portion. In still other embodiments, the invention provides a kit comprising (a) UNG; (b) N,N′-dimethylethylenediamine; (c) ARP; (d) dUTP; (e) a mixture of dATP, dTTP, dCTP, and dGTP; (f) a DNA polymerase; (g) RNAse H; (h) a composite primer, wherein the composite primer comprises a 5′ RNA portion and a 3′ DNA portion. In yet another embodiment, the invention provides a kit comprising (a) UNG; (b) N,N′-dimethylethylenediamine; (c) ARP; (d) dUTP; (e) a mixture of dATP, dTTP, dCTP, and dGTP; (f) a DNA polymerase; (g) RNAse H; (h) a composite primer, wherein the composite primer comprises a 5′ RNA portion and a 3′ DNA portion (i) MgCl₂ solution; (j) acetic acid solution; and optionally, (k) a stop buffer comprising 1.5M Tris, pH 8.5. In some embodiments, the dUTP and the mixture of dATP, dTTP, dCTP, and cGTP are combined. In some embodiments, the DNA polymerase and RNAse H are provided as a mixture. In some embodiments, the RNA portion of the composite primer is 5′ with respect to the 3′ DNA portion, the 5′ RNA portion is adjacent to the 3′ DNA portion, the RNA portion of the composite primer consists of about 10 to about 20 nucleotides and the DNA portion of the composite primer consists of about 7 to about 20 nucleotides. In still other embodiments, the kit comprises a second, different composite primer. In some embodiments, the RNA portion of the composite primer comprises the following ribonucleotide sequence: 5′-GACGGAUGCGGUCU-3′.

In still other embodiments, the invention provides a kit comprising: (a) UNG; (b) ARP; (c) dUTP; (d) a DNA polymerase; (e) RNAse H; (f) a composite primer, wherein the composite primer comprises a 5′ RNA portion and a 3′ DNA portion. In other embodiments, the kit further comprises (g) MgCl₂ solution; (h) acetic acid solution; and optionally, (i) a stop buffer comprising 1.5M Tris, pH 8.5. In other embodiments, the invention provides a kit comprising (a) UNG; (b) an agent capable of labeling an abasic site (for example, Alexa Fluor 555 or an aminooxy-modified Alexa Fluor 555); (c) dUTP; (d) a DNA polymerase; (e) RNAse H; (f) a composite primer, wherein the composite primer comprises a 5′ RNA portion and a 3′ DNA portion. In some embodiments, the DNA polymerase and RNAse H are provided as a mixture. In some embodiments, the RNA portion of the composite primer is 5′ with respect to the 3′ DNA portion, the 5′ RNA portion is adjacent to the 0.3′ DNA portion, the RNA portion of the composite primer consists of about 10 to about 20 nucleotides and the DNA portion of the composite primer consists of about 7 to about 20 nucleotides. In still other embodiments, the kit comprises a second, different composite primer. In some embodiments, the RNA portion of the composite primer comprises the following ribonucleotide sequence: 5′-GACGGAUGCGGUCU-3′.

Kits may also include one or more suitable buffers (as described herein). One or more reagents in the kit can be provided as a dry powder, usually lyophilized, including excipients, which on dissolution will provide for a reagent solution having the appropriate concentrations for performing any of the methods described herein. Each component can be packaged in separate containers or some components can be combined in one container where cross-reactivity and shelf life permit.

The kits of the invention may optionally include a set of instructions, generally written instructions, although electronic storage media (e.g., magnetic diskette or optical disk) containing instructions are also acceptable, relating to the use of components of the methods of the invention for the intended methods of the invention, and/or, as appropriate, for using the products for purposes such as, for example preparing a hybridization probe, expression profiling, preparing a microarray, or characterizing a nucleic acid. The instructions included with the kit generally include information as to reagents (whether included or not in the kit) necessary for practicing the methods of the invention, instructions on how to use the kit, and/or appropriate reaction conditions.

The component(s) of the kit may be packaged in any convenient, appropriate packaging. The components may be packaged separately, or in one or multiple combinations.

The relative amounts of the various components in the kits can be varied widely to provide for concentrations of the reagents that substantially optimize the reactions that need to occur to practice the methods disclosed herein and/or to further optimize the sensitivity of any assay.

Applications using the Labeling and/or Fragmentation and/or Immobilization Methods of the Invention

The methods and compositions of the invention can be used for a variety of purposes. For purposes of illustration, methods of producing a hybridization probe, characterizing and/or quantitating nucleic acid, detecting a mutation, preparing a subtractive hybridization probe, detection (using the hybridization probe), and determining a gene expression profile, using the labeled and/or fragmented nucleic acids generated by the methods of the invention, are described.

Immobilized polynucleotides, for example on a microarray, prepared according to any of the methods of the invention, are also useful for methods of analyzing and characterizing nucleic acids, including methods of hybridizing nucleic acids, methods of characterizing and/or quantitating nucleic acids, methods of detecting a mutation in a nucleic acids, and methods of determining a gene expression profile, as described below, and these applications likewise apply to immobilized polynucleotides.

Method of Producing a Hybridization Probe

The labeled polynucleotides obtained by the methods of the invention are useful as a hybridization probe. Accordingly, in one aspect, the invention provides methods for generating hybridization probes, comprising generating labeled polynucleotides using any of the methods described herein, and using the labeled polynucleotides as a hybridization probe. In another embodiment, the invention provides methods for generating a hybridization probe, comprising generating labeled polynucleotide fragments using any of the methods described herein, and using the labeled polynucleotide fragments as a hybridization probe. The labeled polynucleotide (or polynucleotide fragments) can be produced from any template known in the art, including RNA, DNA, genomic DNA (including global genomic DNA amplification), and libraries (including cDNA, genomic or subtractive hybridization library). The invention also provides methods of hybridizing using the hybridization probes described herein.

Characterization of Nucleic Acids

The labeled and/or fragmented nucleic acids obtained by the methods of the invention are amenable to further characterization.

The labeled and/or fragmented nucleic acids (i.e., products of any of the methods described herein), can be analyzed using, for example, probe hybridization techniques known in the art, such as Southern and Northern blotting, and hybridizing to probe arrays. They can also be analyzed by electrophoresis-based methods, such as differential display and size characterization, which are known in the art.

In one embodiment, the methods of the invention are utilized to generate labeled and/or fragmented nucleic acids, and analyze the labeled and/or fragmented nucleic acids by contact with a probe. The labeled and/or fragmented nucleic acid can be produced from any template known in the art, including RNA, DNA, genomic DNA (including global genomic DNA amplification), and libraries (including cDNA, genomic or subtractive hybridization library).

In one embodiment, the methods of the invention are utilized to generate labeled and/or fragmented nucleic acids which are analyzed (for example, detection and/or quantification) by contacting them with, for example, microarrays (of any suitable substrate, which includes glass, chips, plastic), beads, or particles, that comprise suitable probes such as cDNA and/or oligonucleotide probes. Thus, the invention provides methods to characterize (for example, detect and/or quantify and/or identify) a labeled and/or fragmented nucleic acid by analyzing the labeled products, for example, by hybridization of the labeled products to, for example, probes immobilized at, for example, specific locations on a solid or semi-solid substrate, probes immobilized on defined particles (including beads, such as Bead Array, Illumina), or probes immobilized on blots (such as a membrane), for example arrays, or arrays of arrays. Immobilized probes include immobilized probes generated by the methods described herein, and also include at least the following: cDNA and synthetic oligonucleotides, which can be synthesized directly on the substrate.

Other methods of analyzing labeled products are known in the art, such as, for example, by contacting them with a solution comprising probes, followed by extraction of complexes comprising the labeled products and probes from solution. The identity of the probes provides characterization of the sequence identity of the products, and thus by extrapolation can also provide characterization of the identity of a template from which the products were prepared (for example, the identity. of an RNA in a solution). For example, hybridization of the labeled products is detectable, and the amount of specific labels that are detected is proportional to the amount of the labeled products prepared from a specific RNA sequence of interest. This measurement is useful for, for example, measuring the relative amounts of the various RNA species in a sample, which are related to the relative levels of gene expression, as described herein. The amount of labeled products (as indicated by, for example, detectable signal associated with the label) hybridized at defined locations on an array can be indicative of the detection and/or quantification of the corresponding template RNA species in the sample.

Methods of characterization include sequencing by hybridization (see, e.g., Dramanac, U.S. Pat. No. 6,270,961) and global genomic hybridization (also termed comparative genome hybridization) (see, e.g., Pinkel, U.S. Pat. No. 6,159,685).

In another aspect, the invention provides a method of quantitating labeled and/or fragmented nucleic acids comprising use of an oligonucleotide (probe) of defined sequence (which may be immobilized, for example, on a microarray). Mutation detection utilizing the methods of the invention

The labeled and/or fragmented nucleic acids generated according to the methods of the invention are also suitable for analysis for the detection of any alteration in the template nucleic acid sequence (from which the labeled and/or fragmented nucleic acids are synthesized), as compared to a reference nucleic acid sequence which is identical to the template nucleic acid sequence other than the sequence alteration. The sequence alterations may be sequence alterations present in the genomic sequence or may be sequence alterations which are not reflected in the genomic DNA sequences, for example, alterations due to post transcriptional alterations, and/or mRNA processing, including splice variants. Sequence alterations (interchangeably called “mutations”) include deletion, substitution, insertion and/or transversion of one or more nucleotide.

Accordingly, the invention provides methods of detecting presence or absence of a mutation in a template, comprising: (a) generating a labeled polynucleotide, or fragments thereof, by any of the methods described herein; and (b) analyzing the labeled polynucleotide, or fragments thereof, whereby presence or absence of a mutation is detected. In some embodiments, the labeled polynucleotide, or fragments thereof, is compared to a labeled reference template, or fragments thereof. Step (b) of analyzing the labeled polynucleotide, or fragments thereof, whereby presence or absence of a mutation is detected, can be performed by any method known in the art. In some embodiments, probes for detecting mutations are provided as a microarray.

Any alteration in the test nucleic acid sequence, such as base substitution, insertions or deletion, could be detected using this method. The method is expected to be useful for detection of specific single base polymorphism, SNP, and the discovery of new SNPs.

Other art recognized methods of analysis for the detection of any alteration in the template nucleic acid sequence, as compared to a reference nucleic acid sequence, are suitable for use in the methods of the present invention. For example, essentially any hybridization-based method of detection of mutations is suitable for use with the labeled and/or fragmented nucleic acids produced by the methods of the invention.

Determination of Gene Expression Profile

The labeled and/or fragmented nucleic acids produced by the methods of the invention are particularly suitable for use in determining the levels of expression of one or more genes in a sample. As described above, labeled and/or fragmented nucleic acids can be detected and quantified by various methods, as described herein and/or known in the art. Since RNA is a product of gene expression, the levels of the various RNA species, such as mRNAs, in a sample is indicative of the relative expression levels of the various genes (gene expression profile). Thus, determination of the amount of RNA sequences of interest present in a sample, as determined by quantifying products (for example amplification products) of the sequences, provides for determination of the gene expression profile of the sample source.

Accordingly, the invention provides methods of determining gene expression profile in a sample, said method comprising: amplifying single stranded (or double stranded) product from at least one RNA sequence of interest in the sample, using any of the methods described herein, wherein non-canonical nucleotides are incorporated during synthesis of a polynucleotide; labeling and/or fragmenting the polynucleotide comprising the non-canonical nucleotide; and determining amount of labeled and/or fragmented nucleic acids produced from each RNA sequence of interest, wherein each said amount is indicative of amount of each RNA sequence of interest in the sample, whereby the expression profile in the sample is determined.

Accordingly, the invention provides of determining gene expression profile in a sample, said method comprising: (a) generating labeled polynucleotide or fragments thereof from at least one polynucleotide template in the sample using any of the methods described herein; and (b) determining amount of labeled polynucleotide or fragments thereof of each polynucleotide template, wherein each said amount is indicative of amount of each polynucleotide template in the sample, whereby the gene expression profile in the sample is determined.

It is understood that amount of labeled and/or fragmented nucleic acids produced (and thus the amount of product) may be determined using quantitative and/or qualitative methods. Determining amount of labeled and/or fragmented nucleic acids includes determining whether labeled and/or fragmented nucleic acids are present or absent. Thus, an expression profile can include information about presence or absence of one or more RNA sequence of interest. “Absent” or “absence” of product, and “lack of detection of product” as used herein includes insignificant, or de minimus levels.

The methods of expression profiling are useful in a wide variety of molecular diagnostics, and especially in the study of gene expression in essentially any cell (including a single cell) or cell population. A cell or cell population (e.g. a tissue) may be from, for example, blood, brain, spleen, bone, heart, vascular, lung, kidney, pituitary, endocrine gland, embryonic cells, tumors, or the like. Expression profiling is also useful for comparing a control (normal) sample to a test sample, including test samples collected at different times, including before, after, and/or during development, a treatment, and the like.

Methods of Preparing a Subtractive Hybridization Probe

The labeled and/or fragmented nucleic acids methods of the invention are particularly suitable for use in preparation of labeled and/or fragmented subtractive hybridization probes. For example, two nucleic acid populations, one sense and one antisense, can be allowed to mix together with one population present in molar excess (“driver”). Sequence present in both populations will form hybrids, while sequences present in only one population remain single-stranded. Thereafter, various well-known techniques are used to separate the unhybridized molecules representing differentially expressed sequences. See, e.g., Hamson et al., U.S. Pat. No. 5,589,339; Van Gelder, U.S. Pat. No. 6,291,170. Labeled and/or fragmented subtractive hybridization probe is then labeled and/or fragmented according to the methods of the invention described herein.

Comparative Hybridization

In another aspect, the invention provides methods for comparative hybridization (such as comparative genomic hybridization), said method comprising: (a) preparing a first population of labeled polynucleotides or fragments thereof from a first template polynucleotide sample using any of the methods described herein; (b) comparing hybridization of the first population to at least one probe with hybridization of a second population of labeled polynucleotides or fragments thereof. In some embodiments, the at least one probe is a chromosomal spread. In still other embodiments, the at least one probe is provided as a microarray. In some embodiments, the first and second population comprise detectably different labels. In other embodiments, the second population of labeled polynucleotides, or fragments thereof, are prepared from a second polynucleotide sample using any of the methods described herein. The method according to claim 57, wherein the first population and second population comprise detectably different labels. In some embodiments, step (b) of comparing comprises determining amount of said products, whereby the amount of the first and second polynucleotide templates is quantified.

In some embodiments, comparative hybridization comprises preparing a first population of labeled polynucleotides (which can be polynucleotide fragments) according to any of the methods described herein, wherein the template from which the first population is synthesized is genomic DNA. A second population of labeled polynucleotides (to which the first population is desired to be compared) is prepared from a second genomic DNA template. The first and second populations are labeled with different labels. The hybridized first and second populations are mixed, and hybridized to an array or chromosomal spread. The different labels are detected and compared.

The following Examples are provided to illustrate, but not limit, the invention.

### EXAMPLES

### Example 1

### Demonstration of Fragmentation and Labeling with Biotin of an Abasic Site of a Synthetic Oligonucleotide

A synthetic 75mer oligodeoxynucleotide with a single deoxyuridine incorporated at the 49th position from the 5′ end (Sequence 1) was obtained from Operon (Alameda, Calif.) and dissolved in TE buffer (10 mM Tris/1 mM EDTA, pH 8.0) at a concentration of 0.4 mg/mL.

Sequence 1:

(SEQ ID NO:1)

5′-GGA CCA CCG TTC CGC CGA CCA GAC TCT GCA TAT CTT
CCG CCA TCC CGG UGA CCA TAC CGT AAA AAA AAA AAA
AAA-3′.

Uracil was removed (creating an abasic site) by mixing 5 μL of the oligonucleotide stock with 35 μL of Isotherm® buffer (Epicentre, Madison, Wis.) and 2 Units of UNG (Epicentre, Madison, Wis.), and incubating the mixture at 37° C. for 60 minutes in a thin-walled polypropylene tube in a thermal cycler. Next, the oligonucleotide comprising an abasic site was fragmented (cleaved at the phosphodiester backbone at the abasic site) at the abasic site by incubating the mixture at 99° C. for 30 minutes. The cleaved oligonucleotide product was purified with a QIAquick Nucleotide Removal Kit (Qiagen, Valencia, Calif.) following the manufacturer's instructions, and recovered in approximately 35 μL of water. The fragmented product was labeled by adding 4 μL of 100 mM acetic acid/tetramethylethylenediamine buffer, pH 3.8 (the buffer was prepared by preparing 100 mM acetic acid, and adjusting the pH to 3.8 with TEMED), and 4 μL of ARP (N-(aminooxyacetyl)-N′-(D-biotinoyl) hydrazine, trifluoroacetic acid salt), 22.5 mM in water (Molecular Probes, Eugene, Oreg.), and incubating for 60 minutes at 37° C. The labeling reaction was terminated by adding 5 μL of 1 M Tris buffer, pH 8.5, and the product was again purified as above and recovered in approximately 35 μL water. Appropriate controls were included which omitted either the UNG (data not shown) or the labeling reagent (ARP).

Incorporation of biotin in the product (via the labeling of abasic site with ARP) was detected by mixing 5 μL of product with 3 μL of a 2.5 mg/mL aqueous solution of streptavidin (Sigma, St. Louis. MO) before electrophoresis. The reaction products were analyzed on a PAGE gel (4-20%; InVitrogen, San Diego, Calif.). DNA was visualized using ethidium bromide.

The results of this experiment are shown in the gel photograph of FIG. 4. Lane 1 shows 50 and 100 bp double stranded DNA marker, lane 2 (labeled “no label”) shows the no-label control, lane 3 (labeled “L3.8+Strep”) shows the fragmented and labeled oligonucleotide treated with streptavidin, and lane 4 (labeled “L3.8”) shows the fragmented and labeled oligonucleotide. Note that the single stranded oligonucleotide runs more slowly than the double stranded marker.

Excision of uracil and cleavage of the oligonucleotide were found to be nearly complete in the No Label control (lane 1), as evidenced by appearance of a strong band at ca. 50 nucleotide length and near disappearance of the starting material band at ca. 75 nucleotides. Reaction product treated with label (shown in lane 4, labeled L3.8) was similar in appearance, but the product additionally treated with streptavidin (shown in Lane “L3.8+Strep) was strongly retarded, appearing as fuzzy bands with apparent lengths of several hundred nucleotides. Only a fraction of fragmented product did not react with streptavidin. It was concluded that fragment was nearly completely labeled with ARP.

### Example 2

### Labeling of an abasic site in a Synthetic Oligonucleotide with Biotin without Fragmentation

The experiment in Example 1 was repeated, except that the 99° C. fragmentation step was omitted and the starting oligodeoxynucleotide was Sequence 2. An additional reaction was performed in which the labeling reaction was performed as described in Example 1, except that the buffer was 100 mM acetic acid/tetramethylethylenediamine buffer, pH 6 (the buffer was prepared by preparing 100 mM acetic acid, and adjusting the pH to 6 with TEMED).

Sequence 2:

(SEQ ID NO:2)

5′-GGA CCA CCG TTC CGC CGA CCA GAC UCT GCA TAT CTT
CCG CCA TCC CGG TGA CCA TAC CGT AAA AAA AAA AAA
AAA-3′.

The results of this experiment are shown in FIG. 5. Lane 1 shows molecular weight marker (as described in FIG. 1). Note that the single stranded oligonucleotide runs more slowly than the double stranded marker. “NL” refers to the no-label control, “L6” refers to reactions in which labeling was performed at pH 6, and “L3.8” refers to reactions in which labeling was performed at pH 3.8. Lanes marked “−”,show reaction samples that were not treated with streptavidin, and lanes marked “+” show reaction samples that were treated with streptavidin. The lower arrow marks the expected molecular weight of oligonucleotide not retarded by streptavidin, and the upper arrow marks the expected position of gel retarded product treated with streptavidin.

As shown in lanes “L6” and “L3.8”, nearly all of the product reacted with label could be retarded by streptavidin treatment. Only a fraction of labeled product did not react with streptavidin. It was concluded that fragment was nearly completely labeled with ARP.

By contrast, product not reacted with label (“NL”, or the no label control) was not capable of being retarded by streptavidin treatment.

### Example 3

### Demonstration of Fragmentation and Labeling with Biotin of Ribo-SPIA™ Product.

A mixture of DNA products incorporating deoxyunridine was prepared using Ribo-SPIA™ amplification using commercial total RNA preparation from breast cancer tumor (CLONTECH; cat. no.: 64015-1) as follows:

Primer sequences:

MTB4: 5′-GAC GGA UGC GGU CUC CAG UGU dTdTdT dTdTdT dTdTdT dTdTdT dTdNdN-3′ (SEQ ID NO:3) where dN denotes a degenerate nucleotide (i.e., it can be dA, dT, dC, and dG), and italicized and underlined letters denote ribonucleotides.

MTA4: 5′-GAC GGA UGC GGU CUC CdAdG dTdGdT dTdT-3′ (SEQ ID NO:4) where italicized and underlined letters denote ribonucleotides.

Step 1: First strand cDNA synthesis. Each reaction mixture comprised the following:

4 μl of a 5× buffer (250 mM Tris-HCl, pH 8.3; 375 mM KCl, 15 mM MgCl₂)

MTB4 primer @1 μM

25 mM dNTPs

0.2 μl RNasin Ribonuclease Inhibitor (Promega N2511, 40 u/μl)

1 μl 0.1 M DTT

20 ng of total RNA per reaction

DEPC-treated water to a total volume of 19 μl

The reaction mixtures were pre-incubated at 75° C. for 2 minutes, and then cooled down to 42° C. 1 μL Sensiscript per reaction (Qiagen, Valencia, Calif., Cat No. 205211) was added to each reaction, and the reactions were incubated at 42° C. for 50 minutes.

Step 2: Synthesis of second strand cDNA. 10 μl of the first strand cDNA synthesis reaction mixture was aliquoted to individual reaction tubes. 20 μl of second strand synthesis stock reaction mixture was added to each tube. The second strand synthesis stock reaction mixture contained the following:

2 μL of 10×Klenow reaction buffer (10×buffer: 500 mM Tris-HCl, pH 8.0; 100 mM MgCl₂, 500 mM NaCl)

2U Klenow DNA polymerase (BRL 18012-021)

0.1 μl of AMV reverse transcriptase (BRL 18020-016, 25 U/μl)

0.2 μl of E Coli Ribonuclease H (BRL 18021-014, 4 U/μl)

0.2 μl (25 mM) dNTPs

0 or 0.2 μl of E. coli DNA ligase (BRL 18052-019, 10U/μl)

The reaction mixtures were incubated at 37° C. for 30 minutes. The reactions were stopped by heating to 75° C. for 5 minutes to inactivate the enzymes.

Step 3: Amplification of total cDNA. Amplification was carried out using 1 μl of the second strand cDNA reaction mixture above, using the MTA4 composite primer in the presence of T4 gene 32 protein at 50° C. for 60 min. Each reaction mixture contained the following: 2 μl of 10×buffer (200 mM Tris-HCl, pH 8.5, 50 mM MgCl₂, 1% NP-40) 0.2 μl of dATP, dGTP, dCTP (25 mM) 0.2 μL of a stock containing 20 mM dTTP and 5 mM dUTP 0.2 μl of MTA4 (100 μM) 1 l of the second strand cDNA synthesis mixture 0.1 μl Rnasin 0.1 μl DTT (0.1M) DEPC-treated water to a total volume of 18.8 μl

Reactions were heated to 50° C., 8 Units of Bst DNA Polymerase Large Fragment (New England Biolabs, Beverly, Mass.), 0.02U Hybridase Thermostable Rnase H (Epicentre H39100), and 0.3 μg T4 Gene 32 Protein (USB 70029Z)were added, and the reactions were further incubated at this temperature for 60 min.

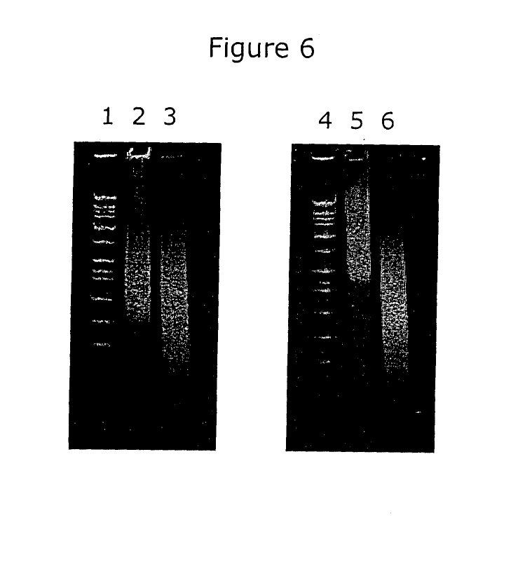

Amplified single stranded DNA product was fragmented and labeled as follows: Approximately 2 μg of product DNA in 40 μL of Isotherm® buffer (Epicentre, Madison, Wis.) was treated with 2 Units of UNG, and fragmented, and labeled as described in Example 1. A control was performed lacking UNG and label (ARP), and without heat treatment. A portion of the fragmented and labeled product was treated with avidin, as described in Example 1. Reaction products were analyzed as described in Example 1 and the results are shown in FIG. 6.

FIG. 6 shows the following:

Lane 1: DNA molecular weight marker as described in Example 1

Lane 2: amplified single stranded DNA product

Lane 3: amplified single stranded DNA product treated with UNG, labeled with biotin, and cleaved by heat treatment

Lane 4: DNA molecular weight marker

Lane 5: streptavidin-treated amplified single stranded DNA product treated with UNG, labeled with biotin, and fragmented by heat treatment

Lane 6: No streptavidin control (contains amplified single stranded DNA product treated with UNG, labeled with biotin, and fragmented by heat treatment, as shown in Lane 3)

Analysis of average size of DNA in the reaction mixtures revealed that the control product of lane 2 was an average length of ca. 400 nucleotides (with the largest products over about 1000 bases). By contrast, the UNG-treated and heat-fragmented product of lane 3 was an average length of 150 nucleotides after UNG and heat treatment, and the largest products (over ca. 1,000 bases) disappeared almost entirely.

An aliquot of the UNG-treated, heat fragmented product was treated with streptavidin, and the results are shown in the Lane 5. Lane 6 shows the no-streptavidin control. Streptavidin treatment resulted in a shift of nearly the entire product band to larger size, indicating virtually complete labeling of the single stranded DNA products treated with UNG, labeled with biotin and fragmented by heat treatment.

### Example 4

### Efficient Labeling and Fragmentation of Ribo-SPIA Product Using a Single Reaction Mixture for Creation of Abasic Sites and Fragmentation at Abasic Sites, with no Intermediate Purification Steps

A mixture of DNA products incorporating deoxyuridine was prepared using total RNA preparation from mouse brain (obtained from the Gladstone Institute, San Francisco Calif.; used with permission) as follows:

Step 1: First strand cDNA synthesis. Each reaction mixture comprised the following:

4 μl of a 5×buffer (250 mM Tris-HCl, pH 8.3; 375 mM KCl, 15 mM MgCl₂)

MTB4 primer @ 0.25 μM

0.2 μL 25 mM dNTPs

0.25 μl RNasin Ribonuclease Inhibitor (Promega N2511, 40 u/μl)

20 ng of total RNA per reaction

DEPC-treated water to a total volume of 19 μl

The reaction mixtures were pre-incubated at 65° C. for 2 minutes, and then cooled down to 42° C. 11L Sensiscript per reaction (Qiagen, Valencia, Calif., Cat No. 205211) was added to each reaction, and the reactions were incubated at 48° C. for 60 minutes, then at 70° C. for 15 minutes.

Step 2: Synthesis of second strand cDNA. The entire 20 μl of the first strand cDNA synthesis reaction mixture was aliquoted to individual reaction tubes, and 20 μl of second strand synthesis stock reaction mixture was added to each tube and mixed. The second strand synthesis stock reaction mixture contained the following:

1 μl of 10×Klenow reaction buffer (10×buffer: 500 mM Tris-HCl, pH 8.0; 100 mM MgCl₂, 500 mM NaCl)

1 mM DTT

1 U/μl of exo-Klenow DNA polymerase (USB catalog number 70057Z)

0.02 U/μl of Ribonuclease H (USB catalog number 70054Z)

0.2 μl (25 mM) dNTPs

0.4 U/μl RNasin (USB catalog number 71571)

water to a total volume of 20 μl.

The reaction mixtures were incubated at 37° C. for 30 minutes. The reactions were stopped by addition of 2 μl of 0.5M EDTA.

Step 3: Amplification of total cDNA: 5 μl of the second strand cDNA reaction mixture was aliquoted into 8 20 μl reaction mixtures. Each reaction mixture contained the following:

2 μl of 10×buffer (200 mM Tris-HCl, pH 8.5, 50 mM MgCl₂, 1% NP-40)

0.2 μl of dATP, dGTP, dCTP (25 mM)

0.2 μL of a stock containing 20 mM dTTP and 5 mM dUTP

0.2 μl of MTA4 (100 μM)

5 μl of the second strand cDNA synthesis mixture

0.1 μl Rnasin

DEPC-treated water to a total volume of 18.8 μl

Reactions were placed on ice and 8 Units of Bst DNA Polymerase Large Fragment (New England Biolabs, Beverly, Mass.), 0.02U Hybridase Thermostable Rnase H (Epicentre H39100), and 0.3 μg T4 Gene 32 Protein (USB 70029Z) were added. Reactions were placed at 50° C. for 60 minutes, then amplification was stopped by heating at 80° C. for 5 minutes.

Removal of uracil (to create abasic sites) and fragmentation of abasic sites was conducted in the same reaction mixture as follows: 76 μl of unpurified amplification reaction product was. mixed with 3.2 μL of N,N′-dimethylethylenediamine buffer (Aldrich Chemical, St. Louis, Mo.; prepared by diluting to 0.5 M solution in water, pH adjusted to 8.5 with HCl) and 4 Units of HK-UNG (Epicentre, Madison Wis.). This mixture was incubated at 37° C. for 60 minutes, then 6.8 μL of 1 M acetic acid in water, 2 μL of 0.2 M MgCl₂ in water, and 8 μL of ARP solution (N-(aminooxyacetyl)-N′-(D-biotinoyl) hydrazine, trifluoroacetic acid salt; 22.5 mM in water; obtained from Molecular Probes, Eugene, Oreg.; catalog no. A-10550) were added. This reaction mixture was incubated for 60 minutes more at 37°, then the reaction mixture was split into two tubes and purified as described in Examples 1-3. Fragmented and labeled reaction product was recovered by elution with water. A portion of the fragmented and labeled product was treated with streptavidin essentially as described in Example 1.

Control reaction mixtures were performed which lacked HK-UNG, or in which the Ribo-SPIA™ product was first purified using a QIAquick PCR purification kit (Qiagen, Valencia, Calif.) following the manufacturer's instructions before fragmentation and labeling as described. A portion of the fragmented and labeled product was treated with streptavidin essentially as described in Example 1.

The following reactions were analyzed using a PAGE gel as described in Example 1 (data not shown):

Lane 1: 50 and 100 bp double stranded DNA molecular weight marker.

Lane 2: No UNG control.

Lane 3: Same as Lane 2, but reacted with streptavidin.

Lane 4: Example 4 reaction product prepared with purified Ribo-SPIA™ product.

Lane 5: Same as Lane 4, reacted with streptavidin.

Lane 6: Example 4 reaction product prepared with unpurified Ribo-SPIA™ product.

Lane 7: Same as Lane 6, reacted with streptavidin.

Analysis of average size of DNA in the reaction mixtures revealed that the no-UNG control product of lane 2 was an average length of ca. 400 nucleotides (with the largest products over about 1000 bases. An aliquot of the no-UNG control product was treated with streptavidin. Streptavidin treatment did not result in a shift of the product band to larger sizes, indicating that the single stranded DNA products were not labeled with biotin, as expected, and that nonspecific interactions between streptavidin and DNA do not cause a shift on the gel.

By contrast, the UNG-treated and dimethylethylenediamine-fragmented product of lanes 4 and 6 was an average length of about 250 nucleotides after UNG and dimethylethylenediamine treatment, and the largest products (over ca. 1,000 bases) disappeared almost entirely. No difference in product length was observed between UNG-treated and dimethylethylenediamine-fragmented product prepared using unpurified Ribo-SPIA single stranded DNA (lane 6) and UNG-treated and dimethylethylenediamine-fragmented product prepared using purified Ribo-SPIA single stranded DNA (lane 4).

Aliquots of UNG-treated and dimethylethylenediamine-fragmented product prepared using unpurified Ribo-SPIA single stranded DNA (lane 6) and UNG-treated and dimethylethylenediamine-fragmented product prepared using purified Ribo-SPIA single stranded DNA (lane 4) were treated with streptavidin, and the results are shown in Lanes 5 and 7, respectively. Streptavidin treatment resulted in a shift of nearly the entire product band to larger size, indicating virtually complete labeling of the single stranded DNA products treated with UNG, labeled with biotin and fragmented by dimethylethylenediamine treatment.

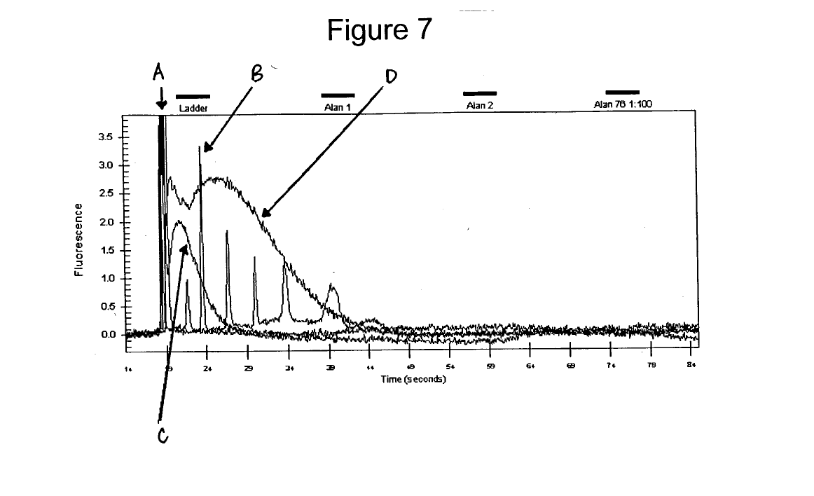

Aliquots of no-UNG treatment control product (corresponding to that of lane 2, above) and UNG-treated and dimethylethylenediamine-fragmented product prepared using purified Ribo-SPIA single stranded DNA (corresponding to that of lane 4, above) were further analyzed by gel electrophoresis using an Agilent Bioanalyzer (Agilent, Mountain View, Calif.). FIG. 7 shows the superimposed resulting electropherograms as follows:

The closely spaced peaks at 19 seconds (“see”) (marked with “A”) are an internal marker included in all samples. The closely matching elution times serve to demonstrate that the instrument is performing reproducibly.

The sharp peak at 22 seconds is a synthetic single-stranded 75mer oligonucleotide used as a size marker (marked with “B”).

The broader peak centered at 21 seconds is the UNG-treated and dimethylethylenediamine-fragmented product prepared using purified Ribo-SPIA single stranded DNA (marked with “C”); material from Lane 6 appeared very similar.

The much broader peak extending to about 42 seconds is the un-fragmented control (no-UNG treatment control product) (marked with “D”).

For comparison, a series of RNA markers (Ambion, Austin Tex.) are also shown in FIG. 7. Marker sizes are 0.2, 0.5, 1, 2, 4, and 6 kb (running at about 21, 23.5, 27, 30, 34, and 39 seconds, respectively).

Both the conventional gel and the Bioanalyzer results establish that the UNG-treated Ribo-SPIA™ products are fragmented compared to a no-UNG-treated control. The difference is much more dramatic in the Bioanalyzer traces because the conventional gel was stained with ethidium bromide, which does not stain small single-stranded DNA well compared to the stain used in the Bioanalyzer.

### Example 5

### Labeling of Ribo-SPIA™ Product with an Aminooxy-Derivatized Dye

The hydrazide of Alexa Fluor 555 (“AF555” or “Alexa Fluor hydrazide”) (Molecular Probes, Eugene, Oreg.) was converted to the aminooxy derivative Alexa Fluor 555-NHNHCOCH₂ONH₂ (“AF555-aminooxy”) using the synthesis protocol disclosed in Ide et al, Biochemistry 32: 8276-83 (1993) (shown as the conversion of compound 2 to compound 5) The starting material shown in Ide as compound 2, BOC-aminooxy)acetic acid is available from Aldrich. The final product was purified using HPLC, and the identity of the product was verified by HPLC and mass spectrometry, which showed a mass that was 73 mass units higher than the starting material.

The aminooxy derivatized dye was dissolved in water to give a 2.1 mM solution. An aliquot was diluted at 1:1000 in water and analyzed on a Beckman DU520 spectrophotometer. The aminooxy derivatized dye retained a UV spectrum identical to unmodified Alexa Fluor 555 (data not shown).

Single stranded amplified DNA product containing dUTP was prepared from Universal Human Reference RNA (Strategene, catalog number A740000) (reaction “U”) or Human Universal Reference Total RNA (Clontech catalog number 64115-1) (reaction “C”), essentially as described in Example 4. Single stranded DNA product (termed “Ribo-SPIA™” product) was then purified using a QIAquick column as described in Example 4.

Purified Ribo-SPIA™ product was labeled as follows: Approximately 10 μg of Ribo-SPIA™ DNA product from reactions U or C was concentrated to 80 μL in water using a SpeedVac. HK-UNG (10 Units; Epicentre; Madison Wis.) and 8 μL of 10×Isotherm® buffer (Epicentre; Madison Wis.) were added to each product and the mixtures were incubated at 37° C. for 60 minutes. Each reaction mixture was then split into two 0.2 mL tubes. One tube of each sample received 1.7 μL of 1 M acetic acid and 3 μL of Alexa Fluor 555 hydrazide in water (7.1 mM or 21.3 nmol total); the other tube received 1.7 μL of 1 M acetic acid plus 10.2 μL of the aminooxy derivative of Alexa Fluor 555 in water (2.1 mM or 21.4 nmol total). After a further incubation at 37° C. for 60 minutes and storage at −20° C. overnight, 5 μL of 1 M Tris pH 8.5 was added to each tube. All products were purified using QIAquick PCR columns as described above, and each product was eluted into 60 μL of water.

Incorporation of dye into fragmented Ribo-SPIA product was analyzed by comparison of dye absorbance at 551 nm to DNA absorbance at 260 nm, using an extinction coefficient of 150,000 for dye and assuming 1 OD of DNA=33 μg/mL. The results of this analysis were expressed as pmol dye/ug DNA and are shown in the column titled “Dye” in Table 1.

TABLE 1

| Sample | Dye (pmol dye/μg DNA) | Fluorescence |
|---|---|---|
| C + AF555 hydrazide | 12.7 | 0.098 × 10⁶ |
| C + AF555 aminooxy | 40.6 | 2.9 × 10⁶ |
| U + AF555 hydrazide | 9.2 | 0.094 × 10⁶ |
| U + AF555 aminooxy | 37.8 | 2.8 × 10⁶ |

The fluorescence intensity of incorporated dye in fragmented Ribo-SPIA product was further analyzed as follows. 0.5 ug of sample in 15 ul of water was diluted in 4 volumes of GeneTac hybridization buffer (Genomic Solutions, Ann Arbor Mich.), re-purified using QIAquick columns as described above, and purified product was reduced to 8 ul under vacuum. A 2 ul aliquot was diluted with 160 ul of water. Fluorescence of duplicate 80 ul aliquots was measured using a Wallac Victor2 fluorometer (Ex=544 nm; Em=595 nm), and the results were averaged. The results of this analysis are shown in the column titled “Fluorescence” in Table 1.

Both dye absorbance and fluorometry analysis reveal that dye was incorporated into Ribo-SPIA product. These results demonstrate that single stranded DNA products containing abasic sites prepared using UNG treatment can be labeled using commercially available dye-containing hydrazide reagents, such as Alexa Fluor 555 hydrazide.

3.2-4.1-fold more dye was incorporated when the aminooxy-derivatized Alexa 555 was used, compared with dye incorporated using the unmodified Alexa-555-hydrazide dye. These results demonstrate that labeling is more efficient when the Alexa 555 dye is converted into an aminooxy derivative.

About 30 fold more fluorescence was detected from the Alexa-555-aminooxy (derivatized)-labeled samples compared to the Alexa-555 hydrazide (unmodified)-labeled samples. Thus, the aminooxy derivative of Alexa 555 dye shows greater brightness.

We note that these samples were subjected to additional purification. The higher fluorescence intensity ratio may indicate that some of the dye in the Alexa 555-hydrazide-labeled samples was not attached covalently and was removed by the additional purification step prior to the fluorescence analysis. Alternatively, or in addition, the fluorescence from the dye moiety in the aminooxy derivatives may be less quenched by interaction with DNA because of the longer linker present in the Alexa 555-aminooxy derivative.

### Example 6

### Detection of Hybridized Fragmented and Labeled Polynucleotides on a Microarray

Total mRNAs were amplified from total RNA from rat brain and rat kidney (Ambion, Austin, Tex., Cat. Nos 7912 and 7926), fragmented, and labeled with biotin as described in the Example 4 control reaction in which the Ribo-SPIA™ product was purified before fragmentation and labeling. Fragmented and labeled probes were prepared for hybridization as follows: 2 μg aliquots of each fragmented and labeled product in 65 μL of water were mixed with 65 μL of formamide, denatured by heating for 2 minutes at 99° C. in a 0.2 mL thin-wall PCR tube, then chilled on ice. An equal amount of 2×GeneTAC buffer (Genomic Solutions, Inc., Ann Arbor, Mich.) was added and the mixtures were applied to CodeLink microarrays (Uniset Rat 1, Part # 300012-03, Motorola Life Sciences, Inc., Northbrook Ill.) and allowed to hybridize following manufacturer's instructions. Post-hybridization processing utilized two 30 minute incubations at 46° C. rather than one incubation for one hour, but otherwise also followed manufacturer's instructions. Detection utilized a 1:100 dilution of Streptavidin-Alexa 647 as described by the manufacturer. Slides were scanned on an Axon GenePix 6000 scanner (Axon Instruments, Inc., Union City, Calif.).

Different spot patterns were observed depending on the starting mRNA sample (i.e., brain vs. kidney), indicating that expression analysis using labeled and fragmented polynucleotides prepared according to the methods of the invention can detect differential expression of RNA in different tissues. The wide range of signal intensities observed indicated that binding is specific for different capture sequences immobilized on different spots, rather than nonspecific binding to DNA on surfaces.

In a further experiment, total RNA from rat kidney (Ambion, Austin, Tex., Cat. No 7926) was amplified, fragmented, and labeled with biotin in duplicate as described in the Example 4 control reaction in which the SPIA™ product was purified before fragmentation and labeling. Probes were prepared for hybridization as follows:2 μg aliquots of each fragmented and labeled product in 65 μL of water were mixed with 65 μL of formamide, denatured by heating for 2 minutes at 99° C. in a 0.2 mL thin-wall PCR tube, then chilled on ice. An equal amount of 2×GeneTAC buffer (Genomic Solutions, Inc., Ann Arbor, Mich.) was added and the mixtures were applied to CodeLink microarrays (Uniset Rat 1, Part # 300012-03, Motorola Life Sciences, Inc., Northbrook Ill.) and allowed to hybridize following manufacturer's instructions. Post-hybridization processing utilized two 30 minute incubations at 46° C. rather than one incubation for one hour, but otherwise also followed manufacturer's instructions. Detection utilized a 1:100 dilution of Streptavidin-Alexa 647 as described by the manufacturer. Slides were scanned on an Axon GenePix 6000 scanner (Axon Instruments, Inc., Union City, Calif.).

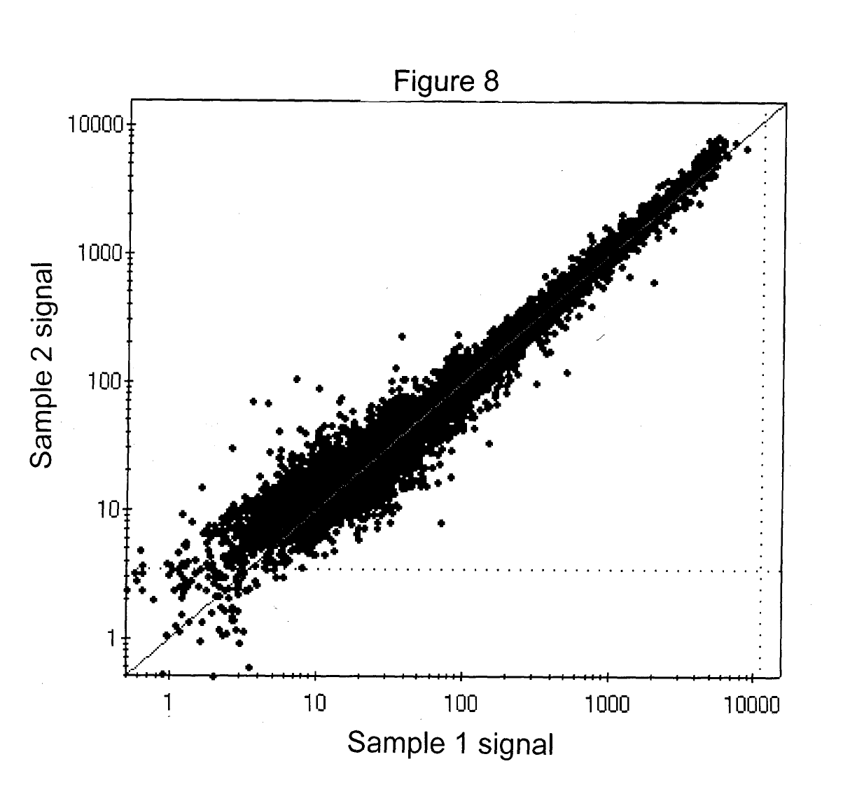

Hybridization intensities were compared between the duplicate hybridizations. The correlation was calculated using the Pearson correlation coefficient calculated using the Codelink (TM) System Software available from Motorola Life Science. The correlation observed between the two independent hybridization reactions is shown in FIG. 8, where the intensities observed for each spot on the arrays are plotted against each other. A useful signal range (dynamic range) of at least three orders of magnitude was obtained, demonstrating that the fragmentation and labeling reaction incorporated enough biotin label to enable detection of gene expression over a large range. The observation of signals three orders of magnitude over background demonstrated that binding to spots on the array is specific for sequences immobilized on individual spots. Good correlation between duplicate arrays (correlation coefficient r=0.98) further confirmed the specificity of the entire amplification and detection process.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

What is claimed is:

1. A method for labeling and fragmenting a polynucleotide, said method comprising:
(a) synthesizing a polynucleotide from a polynucleotide template in the presence of a non-canonical nucleotide, whereby a polynucleotide comprising the non-canonical nucleotide is generated;
(b) cleaving a base portion of the non-canonical nucleotide from the synthesized polynucleotide with an enzyme capable of cleaving the base portion of the non-canonical nucleotide, whereby an abasic site is generated;
(c) cleaving a phosphodiester backbone of the polynucleotide comprising the abasic site at the abasic site; and
(d) labeling the polynucleotide at the abasic site;
whereby a labeled polynucleotide fragment is generated.

2. The method of claim 1, wherein the non-canonical nucleotide is selected from the group consisting of dUTP, dITP, and 5-OH-Me-dCTP.

3. The method of claim 1, wherein the enzyme capable of cleaving a base portion of the non-canonical nucleotide is an N-glycosylase.

4. The method of claim 1, wherein the enzyme capable of cleaving a base portion of the non-canonical nucleotide is selected from the group consisting of Uracil N-Glycosylase (UNG), hypoxanthine-N-Glycosylase, and hydroxy-methyl cytosine-N-glycosylase.

5. The method of claim 1, wherein the non-canonical nucleotide is dUTP and the enzyme capable of cleaving a base portion of the non-canonical nucleotide is Uracil N-Glycosylase.

6. The method of claim 1, wherein the phosphodiester backbone is cleaved with an enzyme or an amine.

7. The method of claim 1, wherein the phosphodiester backbone is cleaved with N,N′-dimethylethylenediamine or AP endonuclease.

8. The method of claim 1, wherein the non-canonical nucleotide is dUTP, the enzyme capable of cleaving a base portion of the non-canonical nucleotide is Uracil N-Glycosylase, and the phosphodiester backbone is cleaved with N,N′-dimethylethylenediamine.

9. The method of claim 1, wherein the phosphodiester backbone is cleaved 3′ to the abasic site.

10. The method of claim 1, wherein the phosphodiester backbone is cleaved 5′ to the abasic site.

11. The method of claim 1, wherein the abasic site is labeled with N-(aminooxyacetyl)-N′-(D-biotinoyl) hydrazine, trifluoroacetic acid salt (ARP), Alexa Fluor 555, or aminooxy-derivatized Alexa Fluor 555.

12. The method of claim 1, wherein the label is capable of reacting with an aldehyde residue at the abasic site.

13. The method of claim 1, wherein the non-canonical nucleotide is dUTP, the enzyme capable of cleaving a base portion of the non-canonical nucleotide is Uracil N-Glycosylase, the phosphodiester backbone is cleaved with N,N′-dimethylethylenediamine, and the abasic site is labeled with ARP.

14. The method of claim 1, wherein the polynucleotide template comprises DNA or RNA.

15. The method of claim 1, wherein the polynucleotide template is selected from the group consisting of RNA, mRNA, cDNA, and genomic DNA.

16. The method of claim 1, wherein the polynucleotide comprising a non-canonical nucleotide is single stranded.

17. The method of claim 1, wherein the polynucleotide comprising a non-canonical nucleotide is double-stranded.

18. The method of claim 1, wherein the polynucleotide comprising the non-canonical nucleotide is synthesized using a method comprising the following steps of:
(a) extending a composite primer in a complex comprising:
(i) a polynucleotide template; and
(ii) the composite primer, said composite primer comprising an RNA portion and a 3′ DNA portion, wherein the polynucleotide template is hybridized to the composite primer; and
(b) cleaving RNA of the annealed composite primer with an enzyme that cleaves RNA from an RNA/DNA hybrid such that another composite primer hybridizes to the template and repeats primer extension by strand displacement, whereby multiple copies of the complementary sequence of the polynucleotide template are produced.

19. The method of claim 18, wherein the complex of part (a) comprises:
(i) a complex of first and second primer extension products, wherein the first primer extension product is produced by extension of a first primer hybridized to a target RNA with at least one enzyme comprising RNA-dependent DNA polymerase activity, wherein the first primer is a composite primer comprising an RNA portion and a 3′ DNA portion; wherein RNA in the complex of first and second primer extension products is cleaved with at least one enzyme that cleaves RNA from an RNA/DNA hybrid such that a composite primer hybridizes to the second primer extension product; and
(ii) the composite primer.

20. The method of claim 1, wherein the polynucleotide comprising a non-canonical nucleotide is synthesized by PCR, reverse transcription, primer extension, limited primer extension, replication, strand displacement amplification (SDA), or nick translation.

21. The method of claim 1, wherein the polynucleotide comprising a non-canonical nucleotide is synthesized using a labeled primer.

22. The method of claim 1, wherein the polynucleotide comprising a non-canonical nucleotide is synthesized using a primer comprising a non-canonical nucleotide.

23. The method of claim 1, wherein the polynucleotide comprising a non-canonical nucleotide is synthesized in the presence of two or more different non-canonical nucleotides, whereby a polynucleotide comprising two or more different non-canonical nucleotide is synthesized.

24. The method of claim 1, wherein the method comprises synthesizing a polynucleotide comprising a non-canonical nucleotide from two or more different polynucleotide templates.

25. The method of claim 1, wherein steps (a), (b) and (c) are performed simultaneously.

26. The method of claim 1, wherein steps (a), (b), (c), and (d) are performed simultaneously.

27. The method of claim 1, wherein steps (b) and (c) are performed simultaneously.

28. The method of claim 1, wherein steps (b), (c), and (d) are performed simultaneously.

29. The method of claim 1, wherein steps (c) and (d) are performed simultaneously.

30. The method of claim 1, wherein step (c) is performed before step (d).

31. The method of claim 1, wherein step (d) is performed before step (c).

32. A method for labeling and fragmenting a polynucleotide, said method comprising:
(a) incubating a reaction mixture, said reaction mixture comprising:
(i) a polynucleotide template; and
(ii) a non-canonical nucleotide; wherein the incubation is under conditions that permit synthesis of a polynucleotide comprising the non-canonical nucleotide, whereby a polynucleotide comprising the non-canonical nucleotide is generated;
(b) incubating a reaction mixture, said reaction mixture comprising:
(i) the polynucleotide comprising the non-canonical nucleotide; and
(ii) an enzyme capable of cleaving a base portion of the non-canonical nucleotide, wherein the incubation is under conditions that permit cleavage of the base portion of the non-canonical nucleotide, whereby a polynucleotide comprising an abasic site is generated;
(c) incubating a reaction mixture, said reaction mixture comprising:
(i) the polynucleotide comprising the abasic site; and
(ii) an agent capable of cleaving a phosphodiester backbone of the polynucleotide comprising the abasic site at the abasic site, wherein the incubation is under conditions that permit cleavage of the phosphodiester backbone of the polynucleotide at the abasic site, whereby a fragment of the polynucleotide is generated;
(d) incubating a reaction mixture, said reaction mixture comprising:
(i) the fragment of the polynucleotide comprising the abasic site; and
(ii) an agent capable of labeling the abasic site, wherein the incubation is under conditions that permit labeling at the abasic site; whereby a labeled polynucleotide fragment is generated.

33. A method for labeling and fragmenting a polynucleotide, said method comprising
(a) incubating a reaction mixture, said reaction mixture comprising:
(i) the polynucleotide comprising the non-canonical polynucleotide of step (a) of claim 1;
(ii) an enzyme capable of cleaving a base portion of the non-canonical nucleotide; and
(iii) an agent capable of cleaving a phosphodiester backbone of the polynucleotide comprising the abasic site at the abasic site, wherein the incubation is under conditions that permit cleavage of the base portion of the non-canonical nucleotide and cleavage of the phosphodiester backbone of the polynucleotide at the abasic site; whereby a fragment of the polynucleotide comprising the abasic site is generated; and
(b) incubating a reaction mixture, said reaction mixture comprising:
(i) the fragment of the polynucleotide comprising the abasic site; and
(ii) an agent capable of labeling the abasic site, wherein the incubation is under conditions that permit labeling at the abasic site, whereby a labeled fragment of the polynucleotide is generated.

34. A method of characterizing a polynucleotide template of interest, comprising:
(a) generating a labeled polynucleotide fragment using the method of any of claims 1, 32, or 33; and
(b) analyzing the labeled polynucleotide fragment.

35. The method of claim 34, wherein step (b) of analyzing the labeled polynucleotide fragment comprises determining amount of said products, whereby the amount of the polynucleotide template present in a sample is quantified.

36. The method of claim 34, wherein step (b) comprises contacting the labeled polynucleotide fragment with at least one probe.

37. The method of claim 36, wherein the at least one probe is provided as a microarray.

38. The method of claim 37, wherein the microarray comprises at least one probe immobilized on a substrate fabricated from a material selected from the group consisting of paper, glass, ceramic, plastic, polypropylene, polystyrene, nylon, polyacrylamide, nitrocellulose, silicon, and optical fiber.

39. The method of claim 38, wherein the probe is immobilized on the substrate in a two-dimensional configuration or a three-dimensional configuration comprising pins, rods, fibers, tapes, threads, beads, particles, microtiter wells, capillaries, and cylinders.

40. A method of determining gene expression profile in a sample, said method comprising:
(a) generating a labeled polynucleotide fragment from at least one polynucleotide template in the sample using the method of any of claims 1, 32, or 33; and
(b) determining amount of labeled polynucleotide fragment from each polynucleotide template, wherein each said amount is indicative of amount of each polynucleotide template in the sample, whereby the gene expression profile in the sample is determined.

41. The method of claim 40, wherein the polynucleotide template is RNA or mRNA.

42. A method of generating hybridization probes, comprising generating a labeled polynucleotide fragment using the method of any of claims according to any of claims 1, 32, or 33.

43. A method of nucleic acid hybridization comprising:
(a) generating a labeled polynucleotide fragment using the method of any of claims according to any of claims 1, 32, or 33; and
(b) hybridizing the labeled polynucleotide fragment with at least one probe.

44. A method for comparative hybridization, said method comprising:
(a) preparing a first population of labeled polynucleotides fragments from a first template polynucleotide sample using the method according to any of claims 1, 32, or 33; and
(b) comparing hybridization of the first population to at least one probe with hybridization of a second population of labeled polynucleotide.

45. The method according to claim 44, wherein the first population and second population comprise detectably different labels.

46. The method according to claim 44, wherein the second population of labeled polynucleotides are prepared from a second polynucleotide sample using the method according to step (a) of claim 44.

47. The method of claim 44, wherein step (b) of comparing comprises determining amount of said products, whereby the amount of the first and second polynucleotide templates is quantified.

48. The method of claim 44, wherein the first and second template polynucleotides comprise genomic DNA.

49. A method for detecting presence or absence of a mutation in a template, comprising:
(a) generating a labeled polynucleotide fragments by any of the methods of claims 1, 32, or 33; and
(b) analyzing the labeled polynucleotide fragment, whereby presence or absence of a mutation is detected.

50. The method of claim 49, wherein the labeled polynucleotide fragment is compared to a reference template.

51. The method of claim 49, wherein the mutation is selected from the group consisting of a base substitution, a base insertion, a base deletion, and a single nucleotide polymorphism.

52. A composition comprising (a) UNG; (b) N,N′-dimethylethylenediamine; and (c) ARP.

53. The composition of claim 52, wherein the composition further comprises (d) dUTP.

54. The composition of claim 53, wherein the composition further comprises: (e) a DNA polymerase; (f) a composite primer, wherein the composite primer comprises a 5′ RNA portion and a 3′ DNA portion; and (g) an agent capable of cleaving RNA from an RNA-DNA hybrid.

55. A composition comprising: (a) a non-canonical nucleotide; (b) an agent capable of cleaving a base portion of a non-canonical nucleotide; (c) an agent capable of cleaving a phosphodiester backbone at an abasic site; (d) an agent capable of labeling an abasic site; and (e) a DNA polymerase; (f) a composite primer, wherein the composite primer comprises a 5′ RNA portion and a 3′ DNA portion; and (g) an agent capable of cleaving RNA from an RNA-DNA hybrid.

56. The composition of claim 55, wherein the composition further comprises: (h) an acetic acid solution; and (i) an MgCl₂ solution.

57. A composition comprising: (a) one or more of: (i) a non-canonical nucleotide; (ii) an agent capable of cleaving a base portion of a non-canonical nucleotide; (iii) an agent capable of cleaving a phosphodiester backbone at an abasic site; and (iv) an agent capable of labeling an abasic site; and (b) a composite primer, wherein the composite primer comprises an RNA portion and a 3′ DNA portion.

58. A composition comprising (a) one or more of: (i) a non-canonical nucleotide; (ii) an agent capable of cleaving a base portion of a non-canonical nucleotide; (iii) an agent capable of cleaving a phosphodiester backbone at an abasic site; and (iv) an agent capable of labeling an abasic site; and (b) an agent capable of cleaving RNA from an RNA-DNA hybrid.

59. The composition of claim 57 or 58, wherein (i) is dUTP.

60. The composition of claim 57 or 58, wherein (ii) is UNG.

61. The composition of claim 57 or 58, wherein (iii) is N,N′-dimethylethylenediamine.

62. The composition of claim 57 or 58, wherein (iv) is ARP.

63. The composition of claim 57, wherein the RNA portion of the composite primer is 5′ with respect to the 3′ DNA portion, the 5′ RNA portion is adjacent to the 3′ DNA portion, the RNA portion of the composite primer consists of about 10 to about 20 nucleotides and the DNA portion of the composite primer consists of about 7 to about 20 nucleotide.

64. The composition of claim 58, wherein the agent that cleaves RNA from an RNA-DNA hybrid is RNAse H.

65. A kit for use in the methods of any of claims 1, 32, or 33, said kit comprising: (a) UNG; (b) N,N′-dimethylethylenediamine; and (c) ARP.

66. The kit of claim 65, wherein the kit further comprises (d) dUTP

67. The kit of claim 66, wherein the kit further comprises: (e) a DNA polymerase; (f) a composite primer, wherein the composite primer comprises a 5′ RNA portion and a 3′ DNA portion; and (g) an agent capable of cleaving RNA from an RNA-DNA hybrid.

68. A kit for use in the methods of any of claims 1, 32, or 33, said kit comprising: (a) a non-canonical nucleotide; (b) an agent capable of cleaving a base portion of a non-canonical nucleotide; (c) an agent capable of cleaving a phosphodiester backbone at an abasic site; (d) an agent capable of labeling an abasic site; and (e) a DNA polymerase; (f) a composite primer, wherein the composite primer comprises a 5′ RNA portion and a 3′ DNA portion; and (g) an agent capable of cleaving RNA from an RNA-DNA hybrid.

69. The kit of claim 68, wherein the kit further comprises: (h) an acetic acid solution; and (i) an MgCl₂ solution.

70. A kit for use in the methods of any of claims 1, 32, or 33, said kit comprising: (a) one or more of: (i) a non-canonical nucleotide; (ii) an agent capable of cleaving a base portion of a non-canonical nucleotide; (iii) an agent capable of cleaving a phosphodiester backbone at an abasic site; and (iv) an agent capable of labeling an abasic site; and (b) a composite primer, wherein the composite primer comprises an RNA portion and a 3′ DNA portion.

71. A kit for use in the methods of any of claims 1, 32, or 33, said kit comprising: (a) one or more of: (i) a non-canonical nucleotide; (ii) an agent capable of cleaving a base portion of a non-canonical nucleotide; (iii) an agent capable of cleaving a phosphodiester backbone at an abasic site; and (iv) an agent capable of labeling an abasic site; and (b) an agent capable of cleaving RNA from an RNA-DNA hybrid.

72. The kit of claim 70 or 71, wherein (i) is dUTP.

73. The kit of claim 70 or 71, wherein (ii) is UNG.

74. The kit of claim 70 or 71, wherein (iii) is N,N′-dimethylethylenediamine.

75. The kit of claim 70 or 71, wherein (iv) is ARP.

76. The kit of claim 70, wherein the RNA portion of the composite primer is 5′ with respect to the 3′ DNA portion, the 5′ RNA portion is adjacent to the 3′ DNA portion, the RNA portion of the composite primer consists of about 10 to about 20 nucleotides and the DNA portion of the composite primer consists of about 7 to about 20 nucleotide.

77. The kit of claim 71, wherein the agent that cleaves RNA from an RNA-DNA hybrid is RNAse H.

78. The kit of claim 70 or 71, wherein (ii) is an enzyme.
